(19) United States
(12) Patent Application Publication
(10) Pub. No.: US 2025/0304933 A1
(43) Pub. Date: Oct. 2, 2025

(54) INDUCIBLE RECOMBINASE SYSTEMS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ross Levine, New York, NY (US); Robert Lyle Bowman, New York, NY (US); Andrew Dunbar, New York, NY (US); Linde Miles, Cincinnati, OH (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 18/864,883

(22) PCT Filed: May 12, 2023
(86) PCT No.: PCT/US2023/066910

§ 371 Date: Nov. 11, 2024

Related U.S. Application Data
(60) Provisional application No. 63/341,312, filed on May 12, 2022.

(51) Int. Cl. C12N 9/22; C12N 15/67

(52) U.S. Cl. CPC C12N 9/22; C12N 15/67; C12N 2800/30

(57) ABSTRACT

Aspects of the disclosure provide compositions and methods for reversible expression of a target gene. In some aspects, the disclosure provides nucleic acids comprising multiple pairs of recombinase sites, each pair having first and second members flanking an inverted expression cassette encoding a target gene.

### CROSS-REFERENCE TO RELATED APPLICATIONS

This application claims priority under 35 U.S.C. § 119(e) to U.S. Provisional Patent Application No. 63/341,312, filed May 12, 2022, which is hereby incorporated by reference in its entirety.

### GOVERNMENT SUPPORT

This invention was made with government support under CA108671 and CA008748, awarded by the National Institutes of Health. The government has certain rights in this invention.

### REFERENCE TO AN ELECTRONIC SEQUENCE LISTING

The contents of the electronic sequence listing (S171570057WO00-SEQ-JIB.xml; Size: 81,028 bytes; and Date of Creation: May 9, 2023) is herein incorporated by reference in its entirety.

### BACKGROUND

Site-specific recombinases have an exceptional ability to excise, integrate, invert, and translocate genomic DNA in living organisms. Recombinase-based technologies have considerably advanced and refined our understanding of the mechanisms underlying many biological processes. The development of new technologies that provide researchers more precise control over these systems could enable the study of cellular processes beyond the scope of conventional approaches.

### SUMMARY

Despite the extensive catalog of genomic alterations revealed by sequencing studies, there remain limited means to functionally model and perturb the process of disease development and progression in experimental systems. Aspects of the present disclosure provide nucleic acid constructs having a generalizable allelic framework that allows for the reversible expression of disease-associated mutations at their endogenous loci. Advantageously, nucleic acid constructs described herein are configured for inducible activation and inactivation of a target gene, providing a means for expanding our understanding of the spatiotemporal dynamics of disease evolution, even at early stages of disease development and regression.

In some aspects, the disclosure provides a nucleic acid comprising: (I) a first nucleic acid segment comprising, in 5′ to 3′ order: (a) first member of a first pair of recombinase sites, each member of the first pair comprising a recognition sequence for a first recombinase; (b) a first member of a second pair of recombinase sites, each member of the second pair comprising a recognition sequence for a second recombinase; (c) a first member of a third pair of recombinase sites, each member of the third pair comprising a recognition sequence for the second recombinase; and (d) a second member of the first pair of recombinase sites; (II) a second nucleic acid segment comprising an inverted expression cassette encoding a target gene; and (III) a third nucleic acid segment comprising, in 5′ to 3′ order: (e) a second member of the second pair of recombinase sites; and (f) a second member of the third pair of recombinase sites. In some embodiments, the nucleic acid comprises, in 5′ to 3′ order: (I), (II), and (III). In some embodiments, the nucleic acid comprises, in 5′ to 3′ order: (III), (II), and (I).

In some embodiments, (b)-(c) of the first nucleic acid segment is not recombined upon exposure to the first recombinase. In some embodiments, (a) and (d) are separated from one another by a distance that prevents recombination of (b)-(c) upon exposure to the first recombinase. In some embodiments, (a) and (d) are separated from one another by fewer than 90 contiguous nucleobases. In some embodiments, (a) and (d) are separated from one another by fewer than 85 contiguous nucleobases. In some embodiments, (a) and (d) are separated from one another by 82 contiguous nucleobases or fewer. In some embodiments, (a) and (d) are separated from one another by between about 40 and about 100 contiguous nucleobases (e.g., 40-95, 50-90, 50-85, 60-85, 65-85, or 70-85 contiguous nucleobases). In some embodiments, (a) and (d) are separated from one another by between about 40 and about 82 contiguous nucleobases (e.g., 40-82, 45-82, 50-82, 55-82, 60-82, 65-82, 70-82, 75-82, 80-82, 40-81, 40-80, 40-70, 40-60, 40-50, 50-80, 50-70, 50-60, 60-80, or 70-80 contiguous nucleobases).

In some embodiments, the first and second members of each of the second and third pairs of recombinase sites are in an orientation opposite one another. In some embodiments, the first and second members of each of the second and third pairs of recombinase sites are in forward and reverse orientations, respectively. In some embodiments, the first and second members of each of the second and third pairs of recombinase sites are in reverse and forward orientations, respectively. In some embodiments, the first and second members of the second pair of recombinase sites are in forward and reverse orientations, respectively; and the first and second members of the third pair of recombinase sites are in reverse and forward orientations, respectively. In some embodiments, the first and second members of the second pair of recombinase sites are in reverse and forward orientations, respectively; and the first and second members of the third pair of recombinase sites are in forward and reverse orientations, respectively.

In some embodiments, the first recombinase is selected from Cre recombinase, Dre recombinase, and Flp recombinase; and the second recombinase is selected from Cre recombinase, Dre recombinase, and Flp recombinase, provided that the first recombinase is different from the second recombinase.

In some embodiments, the first recombinase is a Cre recombinase, and the second recombinase is a Dre recombinase or a Flp recombinase. In some embodiments, the first recombinase is a Dre recombinase, and the second recombinase is a Cre recombinase or a Flp recombinase. In some embodiments, the first recombinase is a Flp recombinase, and the second recombinase is a Cre recombinase or a Dre recombinase. In some embodiments, the first recombinase is a Cre recombinase or a Flp recombinase, and the second recombinase is a Dre recombinase. In some embodiments, the first recombinase is a Dre recombinase or a Flp recombinase, and the second recombinase is a Cre recombinase. In some embodiments, the first recombinase is a Cre recombinase or a Dre recombinase, and the second recombinase is a Flp recombinase.

In some embodiments, the first recombinase is a Cre recombinase, and the second recombinase is a Dre recombinase. In some embodiments, the first recombinase is a Cre recombinase, and the second recombinase is a Flp recombinase. In some embodiments, the first recombinase is a Dre recombinase, and the second recombinase is a Cre recombinase. In some embodiments, the first recombinase is a Dre recombinase, and the second recombinase is a Flp recombinase. In some embodiments, the first recombinase is a Flp recombinase, and the second recombinase is a Cre recombinase. In some embodiments, the first recombinase is a Flp recombinase, and the second recombinase is a Dre recombinase.

In some embodiments, the first and second members of the first pair of recombinase sites are Lox sites, variant Lox sites, Frt sites, or variant Frt sites. In some embodiments, the first and second members of the second pair of recombinase sites are Rox sites, and the first and second members of the third pair of recombinase sites are variant Rox sites. In some embodiments, the first and second members of the second pair of recombinase sites are variant Rox sites, and the first and second members of the third pair of recombinase sites are Rox sites.

In some embodiments, the first and second members of each pair of recombinase sites are in an orientation opposite one another. In some embodiments, the first and second members of the first pair of recombinase sites are in forward and reverse orientations, respectively. In some embodiments, the first and second members of the first pair of recombinase sites are in reverse and forward orientations, respectively. In some embodiments, the target gene encodes a protein of interest. In some embodiments, the target gene encodes a protein comprising a disease-associated mutation.

In some embodiments, the second nucleic acid segment comprises: a first expression cassette encoding a first target gene; and a second expression cassette encoding a second target gene, wherein the first or second expression cassette is the inverted expression cassette. In some embodiments, the first target gene encodes a wild-type protein, and the second target gene encodes a mutational variant of the wild-type protein. In some embodiments, the second target gene encodes a wild-type protein, and the first target gene encodes a mutational variant of the wild-type protein. In some embodiments, the first and second members of the first pair of recombinase sites are in the same orientation. In some embodiments, the first and second members of the first pair of recombinase sites are variant Lox sites, each variant Lox site comprising a different sequence. In some embodiments, the first or second member of the first pair of recombinase sites comprises a Lox71 site. In some embodiments, the first pair of recombinase sites comprises a Lox66 site and a Lox71 site.

In some aspects, the disclosure provides a nucleic acid comprising, in 5′ to 3′ order: (a) a first member of a first pair of recombinase sites, each member of the first pair comprising a recognition sequence for a first recombinase; (b) a first member of a second pair of recombinase sites, each member of the second pair comprising a recognition sequence for a second recombinase; (c) a first member of a third pair of recombinase sites, each member of the third pair comprising a recognition sequence for the second recombinase; (d) a second member of the first pair of recombinase sites; (e) an inverted expression cassette encoding a target gene; (f) a second member of the second pair of recombinase sites; and (g) a second member of the third pair of recombinase sites.

In some embodiments, the first and second members of each pair of recombinase sites are in an orientation opposite one another. In some embodiments, (b)-(c) is not inverted upon exposure to the first recombinase. In some embodiments, (c)-(e) or (d)-(f) is inverted upon exposure to the second recombinase. In some embodiments, the first and second members of the first pair of recombinase sites are in the same orientation upon inversion of (c)-(e) or (d)-(f) by the second recombinase.

In some embodiments, (a) and (d) are separated from one another by a distance that prevents inversion of (b)-(c) upon exposure to the first recombinase. In some embodiments, (a) and (d) are separated from one another by fewer than 90 contiguous nucleobases. In some embodiments, (a) and (d) are separated from one another by fewer than 85 contiguous nucleobases. In some embodiments, (a) and (d) are separated from one another by 82 contiguous nucleobases or fewer.

In some embodiments, the first member of each pair of recombinase sites is in a forward orientation, and the second member of each pair of recombinase sites is in a reverse orientation. In some embodiments, the first member of each pair of recombinase sites is in a reverse orientation, and the second member of each pair of recombinase sites is in a forward orientation.

In some embodiments, the first recombinase is selected from Cre recombinase, Dre recombinase, and Flp recombinase; and the second recombinase is selected from Cre recombinase, Dre recombinase, and Flp recombinase, provided that the first recombinase is different from the second recombinase.

In some embodiments, the first and second members of the first pair of recombinase sites are Lox sites, variant Lox sites, Frt sites, or variant Frt sites. In some embodiments, the first and second members of the second pair of recombinase sites are Rox sites, and the first and second members of the third pair of recombinase sites are variant Rox sites. In some embodiments, the first and second members of the second pair of recombinase sites are variant Rox sites, and the first and second members of the third pair of recombinase sites are Rox sites.

In some embodiments, the target gene encodes a protein of interest. In some embodiments, the target gene encodes a protein comprising a disease-associated mutation.

In some aspects, the disclosure provides a method for regulating expression of a target gene, the method comprising: contacting the nucleic acid with the second recombinase, where the target gene is inverted to a sense orientation by the second recombinase. In some embodiments, the method further comprises contacting the nucleic acid with the first recombinase, where the target gene is excised from the nucleic acid by the first recombinase.

In some embodiments, the contacting is in a cell. In some embodiments, the cell is in a non-human mammal.

In some aspects, the disclosure provides a nucleic acid comprising, in 5′ to 3′ order: (a) a first member of a first pair of recombinase sites, each member of the first pair comprising a recognition sequence for a first recombinase; (b) a first member of a second pair of recombinase sites, each member of the second pair comprising a recognition sequence for the first recombinase; (c) an inverted expression cassette encoding a target gene; (d) a first member of a third pair of recombinase sites, each member of the third pair comprising a recognition sequence for a second recombinase; (e) a second member of the first pair of recombinase sites; (f) a second member of the second pair of recombinase sites; and (g) a second member of the third pair of recombinase sites.

In some embodiments, the first and second members of each pair of recombinase sites are in an orientation opposite one another. In some embodiments, (e)-(f) is not inverted upon exposure to the second recombinase. In some embodiments, (b)-(d) or (c)-(e) is inverted upon exposure to the first recombinase. In some embodiments, the first and second members of the third pair of recombinase sites are in the same orientation upon inversion of (b)-(d) or (c)-(e) by the second recombinase.

In some embodiments, (d) and (g) are separated from one another by a distance that prevents inversion of (e)-(f) upon exposure to the second recombinase. In some embodiments, (d) and (g) are separated from one another by fewer than 90 contiguous nucleobases. In some embodiments, (d) and (g) are separated from one another by fewer than 85 contiguous nucleobases. In some embodiments, (d) and (g) are separated from one another by 82 contiguous nucleobases or fewer.

In some embodiments, the first member of each pair of recombinase sites is in a forward orientation, and the second member of each pair of recombinase sites is in a reverse orientation. In some embodiments, the first member of each pair of recombinase sites is in a reverse orientation, and the second member of each pair of recombinase sites is in a forward orientation.

In some embodiments, the first recombinase is selected from Cre recombinase, Dre recombinase, and Flp recombinase; and the second recombinase is selected from Cre recombinase, Dre recombinase, and Flp recombinase, provided that the first recombinase is different from the second recombinase.

In some embodiments, the first and second members of the third pair of recombinase sites are Lox sites, variant Lox sites, Frt sites, or variant Frt sites. In some embodiments, the first and second members of the first pair of recombinase sites are Rox sites, and the first and second members of the second pair of recombinase sites are variant Rox sites. In some embodiments, the first and second members of the first pair of recombinase sites are variant Rox sites, and the first and second members of the second pair of recombinase sites are Rox sites.

In some embodiments, the target gene encodes a protein of interest. In some embodiments, the target gene encodes a protein comprising a disease-associated mutation.

In some aspects, the disclosure provides a method for regulating expression of a target gene, the method comprising: contacting the nucleic acid with the first recombinase, where the target gene is inverted to a sense orientation by the first recombinase. In some embodiments, the method further comprises contacting the nucleic acid with the second recombinase, where the target gene is excised from the nucleic acid by the second recombinase. In some embodiments, the contacting is in a cell. In some embodiments, the cell is in a non-human mammal.

In some aspects, the disclosure provides a nucleic acid comprising, in 5′ to 3′ order: (a) a first member of a first pair of recombinase sites, each member of the first pair comprising a recognition sequence for a first recombinase; (b) a first member of a second pair of recombinase sites, each member of the second pair comprising a recognition sequence for a second recombinase; (c) a first member of a third pair of recombinase sites, each member of the third pair comprising a recognition sequence for the second recombinase; (d) a second member of the first pair of recombinase sites; (e) a first expression cassette encoding a first target gene; (f) a second expression cassette encoding a second target gene, wherein the first or second expression cassette is an inverted expression cassette; (g) a second member of the second pair of recombinase sites; and (h) a second member of the third pair of recombinase sites.

In some embodiments, the first and second members of the first pair of recombinase sites are in the same orientation. In some embodiments, the first and second members of each of the second and third pairs of recombinase sites are in an orientation opposite one another.

In some embodiments, (b)-(c) is not excised from the nucleic acid upon exposure to the first recombinase. In some embodiments, (c)-(f) or (d)-(g) is inverted upon exposure to the second recombinase. In some embodiments, the first and second members of the first pair of recombinase sites are in an orientation opposite one another upon inversion of (c)-(f) or (d)-(g) by the second recombinase. In some embodiments, the first and second members of the first pair of recombinase sites are in a forward orientation. In some embodiments, the first and second members of the first pair of recombinase sites are in a reverse orientation.

In some embodiments, (a) and (d) are separated from one another by a distance that prevents excision of (b)-(c) upon exposure to the first recombinase. In some embodiments, (a) and (d) are separated from one another by fewer than 90 contiguous nucleobases, fewer than 85 contiguous nucleobases, or 82 contiguous nucleobases or fewer.

In some embodiments, the first recombinase is selected from Cre recombinase, Dre recombinase, and Flp recombinase; and the second recombinase is selected from Cre recombinase, Dre recombinase, and Flp recombinase, provided that the first recombinase is different from the second recombinase. In some embodiments, the first and second members of the first pair of recombinase sites are Lox sites, variant Lox sites, Frt sites, or variant Frt sites. In some embodiments, the first and second members of the first pair of recombinase sites are variant Lox sites, each variant Lox site comprising a different sequence. In some embodiments, the first pair of recombinase sites comprises a Lox66 site and a Lox71 site. In some embodiments, the first and second members of the second pair of recombinase sites are Rox sites, and the first and second members of the third pair of recombinase sites are variant Rox sites. In some embodiments, the first and second members of the second pair of recombinase sites are variant Rox sites, and the first and second members of the third pair of recombinase sites are Rox sites.

In some embodiments, the first target gene encodes a wild-type protein, and the second target gene encodes a mutational variant of the wild-type protein. In some embodiments, the second target gene encodes a wild-type protein, and the first target gene encodes a mutational variant of the wild-type protein. In some embodiments, the first or second target gene encodes a protein comprising a disease-associated mutation.

In some aspects, the disclosure provides a method for regulating expression of a target gene, the method comprising: contacting the nucleic acid with the second recombinase, where the first or second target gene is inverted to a sense orientation by the second recombinase. In some embodiments, the method further comprises contacting the nucleic acid with the first recombinase, wherein the first or second target gene is inverted to an antisense orientation by the first recombinase. In some embodiments, the contacting is in a cell. In some embodiments, the cell is in a non-human mammal.

In some aspects, the disclosure provides a nucleic acid comprising, in 5′ to 3′ order: (a) a first member of a first pair of recombinase sites, each member of the first pair comprising a recognition sequence for a first recombinase; (b) a first member of a second pair of recombinase sites, each member of the second pair comprising a recognition sequence for the first recombinase; (c) a first expression cassette encoding a first target gene; (d) a second expression cassette encoding a second target gene, wherein the first or second expression cassette is an inverted expression cassette; (e) a first member of a third pair of recombinase sites, each member of the third pair comprising a recognition sequence for a second recombinase; (f) a second member of the first pair of recombinase sites; (g) a second member of the second pair of recombinase sites; and (h) a second member of the third pair of recombinase sites.

In some embodiments, the first and second members of each of the first and second pairs of recombinase sites are in an orientation opposite one another. In some embodiments, the first and second members of the third pair of recombinase sites are in the same orientation. In some embodiments, (f)-(g) is not excised from the nucleic acid upon exposure to the second recombinase. In some embodiments, (b)-(e) or (c)-(f) is inverted upon exposure to the first recombinase. In some embodiments, the first and second members of the third pair of recombinase sites are in an orientation opposite one another upon inversion of (b)-(e) or (c)-(f) by the second recombinase. In some embodiments, the first and second members of the third pair of recombinase sites are in a forward orientation. In some embodiments, the first and second members of the third pair of recombinase sites are in a reverse orientation.

In some embodiments, (e) and (h) are separated from one another by a distance that prevents inversion of (f)-(g) upon exposure to the second recombinase. In some embodiments, (e) and (h) are separated from one another by fewer than 90 contiguous nucleobases, fewer than 85 contiguous nucleobases, or 82 contiguous nucleobases or fewer.

In some embodiments, the first recombinase is selected from Cre recombinase, Dre recombinase, and Flp recombinase. In some embodiments, the second recombinase is selected from Cre recombinase, Dre recombinase, and Flp recombinase, provided that the first recombinase is different from the second recombinase. In some embodiments, the first and second members of the third pair of recombinase sites are Lox sites, variant Lox sites, Frt sites, or variant Frt sites. In some embodiments, the first and second members of the third pair of recombinase sites are variant Lox sites, each variant Lox site comprising a different sequence. In some embodiments, the third pair of recombinase sites comprises a Lox66 site and a Lox71 site. In some embodiments, the first and second members of the first pair of recombinase sites are Rox sites, and the first and second members of the second pair of recombinase sites are variant Rox sites. In some embodiments, the first and second members of the first pair of recombinase sites are variant Rox sites, and the first and second members of the second pair of recombinase sites are Rox sites.

In some embodiments, the first target gene encodes a wild-type protein, and the second target gene encodes a mutational variant of the wild-type protein. In some embodiments, the second target gene encodes a wild-type protein, and the first target gene encodes a mutational variant of the wild-type protein. In some embodiments, the first or second target gene encodes a protein comprising a disease-associated mutation.

In some aspects, the disclosure provides a method for regulating expression of a target gene, the method comprising: contacting the nucleic acid with the first recombinase, where the first or second target gene is inverted to a sense orientation by the first recombinase. In some embodiments, the method further comprises contacting the nucleic acid with the second recombinase, wherein the first or second target gene is inverted to an antisense orientation by the second recombinase. In some embodiments, the contacting is in a cell. In some embodiments, the cell is in a non-human mammal.

In some aspects, the disclosure provides a cell comprising a nucleic acid described herein. In some embodiments, the cell further comprises the first recombinase and/or a nucleic acid encoding the first recombinase. In some embodiments, the cell further comprises the second recombinase and/or a nucleic acid encoding the second recombinase. In some embodiments, the cell further comprises an inducer that activates the first and/or second recombinase. In some embodiments, the inducer is a drug or chemical. In some aspects, the disclosure provides a non-human mammal comprising the cell. In some aspects, the disclosure provides a non-human mammal comprising a nucleic acid described herein.

The details of certain embodiments of the invention are set forth in the Detailed Description, as described below. Other features, objects, and advantages of the invention will be apparent from the Examples, Drawings, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1A shows an example configuration of a nucleic acid of the present disclosure.

FIG. 1B shows an example process illustrating inducible activation and inactivation of target gene expression using a nucleic acid having the example configuration shown in FIG. 1A.

FIGS. 1C-1D show example configurations of nucleic acids which may be used in accordance with the example process of FIG. 1B.

FIG. 1E shows an example configuration of a nucleic acid of the present disclosure.

FIG. 1F shows an example process illustrating inducible activation and inactivation of target gene expression using a nucleic acid having the example configuration shown in FIG. 1E.

FIGS. 1G-1H show example configurations of nucleic acids which may be used in accordance with the example process of FIG. 1F.

FIGS. 2A-2G show Jak2^{V617F} deletion abolishes JAK/STAT signaling and abrogates the myeloproliferative neoplasm (MPN) phenotype. FIG. 2A shows a schematic representation of the dual-recombinase Jak2^{V617F} conditional knock-in/knock-out allele (Jak2^{RL}), the Jak2^{RL} knock-in allele following Dre recombination, and the null recombined allele following Cre-mediated deletion; Semi-circles indicate Rox sequences; triangles indicate loxP sequences. FIG. 2B shows a representative western blot depicting phospho-STAT5 abundance of Dre-mediated Jak2^{V617F} knock-in (+Dre) vs. Jak2^{V617F}-deleted (+Dre +Cre) states from isolated splenocytes 7 days following tamoxifen administration in comparison to unrecombined (Unrec.) Jak2^{RL} cells (n=2 biological replicates each from n=3 independent experiments). FIG. 2C shows peripheral blood count trends (weeks 0-24) of MPN (CTRL) vs. tamoxifen (TAM; Jak2^{V617F}-deleted) treated mice: white blood cells (WBC; left panel), hematocrit (Hct; right panel) (n≥10 each; mean±s.e.m.). Gray bar represents duration of TAM pulse/chow administration. Representative of n=2 independent transplants. ****p<0.0001. FIG. 2D shows Kaplan-Meier survival analysis of MPN (CTRL) vs. tamoxifen (TAM; Jak2^{V617F}-deleted) treated mice (n=12 vs. 14 per arm; Log-rank test). Gray bar represents duration of TAM pulse/chow administration. ****p<0.0001. FIG. 2E shows spleen weights of MPN (CTRL) vs. tamoxifen (TAM; Jak2^{V617F}-deleted) treated mice at timed sacrifice (24 weeks) in comparison to wild-type control mice (mean±s.e.m.). Representative of n=2 independent transplants. ****p<0.0001. FIG. 2F shows a heatmap demonstrating fold-change in inflammatory cytokine levels of MPN (CTRL) vs. tamoxifen (TAM; Jak2^{V617F}-deleted) treated mice in comparison to wild-type control mice (n=5 biological replicates per arm). FIG. 2G shows hematoxylin and eosin (H&E) and reticulin stains of bone marrow of MPN (Control) vs. tamoxifen (Jak2^{V617F}-deleted) treated mice from timed sacrifice 24 weeks. Representative micrographs of n=7 individual mouse replicates per arm. All images represented at 400× magnification. Scale bar: 20 μm.

FIGS. 3A-3G show Jak2^{V617F} reversal impairs the fitness of MPN cells, including MPN stem cells. FIG. 3A shows peripheral blood (PB) mutant CD45.2 percent chimerism trend (weeks 0-24) of “early” (3-weeks posttransplant) tamoxifen (TAM; Jak2^{V617F}-deleted) treated (data points at bottom) and “late” (12-weeks post-transplant) tamoxifen treated (data points at middle) mice (n=8 each) in comparison to MPN (CTRL; data points at top; n=6) mice (mean±s.e.m.). Gray bars represent duration of TAM pulse/chow administration. Representative of n=2 independent transplants. **p<0.01, ***p<0.001. FIG. 3B shows bone marrow mutant cell fraction within LSK (Lineage−Sca1+cKit+), granulocytic-monocytic progenitor (GMP; Lineage−cKit+Sca1−Cd34+Fcg+), and megakaryocytic-erythroid progenitor (MEP; Lineage−cKit+Sca1−Cd34−Fcg−) compartments of “early” (3-weeks post-transplant) tamoxifen (TAM; Jak2^{V617F}-deleted) treated and “late” (12-weeks post-transplant) tamoxifen treated mice in comparison to MPN (CTRL) mice at timed sacrifice 24 weeks (n=6-8 individual biological replicates per arm; mean±s.e.m.). Representative of n=2 independent transplants. **p<0.01, ***p<0.001, ****p<0.0001. FIG. 3C shows gene-set enrichment analysis (GSEA) of significant Hallmark gene sets of MPN (CTRL) vs. tamoxifen (TAM; Jak2^{V617F}-deleted) treated LSKs isolated 7 days following initiation of TAM (n=3-4 biological replicates per arm). FIG. 3D shows a volcano plot demonstrating differential gene expression of MPN (CTRL) vs. TAM (Jak2^{V617F}-deleted) treated LSKs 7 days following initiation of TAM (n=3-4 biological replicates per arm). FIG. 3E shows GMP and MEP stem cell frequencies of MPN (CTRL) vs. tamoxifen (TAM; Jak2^{V617F}-deleted) treated mice 7 days following initiation of TAM (n=8 biological replicates per arm across 2 independent transplants; mean±s.e.m.). FIG. 3F shows a row normalized heatmap of RNA-sequencing data of key erythroid differentiation factor genes from harvested MEPs at baseline (CTRL), day 3 (D3) and day 7 (D7) following initiation of TAM (Jak2^{V617F} deletion). FIG. 3G shows a HOMER motif analysis from Assay for Transposase-Accessibility Chromatin (ATAC) sequencing (ATAC-seq) data demonstrating increased accessibility of Cebp motif signatures with concomitant decreased accessibility of Gata motif signatures in isolated cKit+Jak2^{RL} bone marrow cells isolated 7 days following initiation of TAM in comparison to control MPN cells (n=3 biological replicates per arm).

FIGS. 4A-4J show differential efficacy of Jak2^{V617F} deletion compared to JAK inhibitor therapy. FIG. 4A shows a scatter plot depicting log 2FoldChange of ruxolitinib (RUX) treated vs. tamoxifen (TAM; Jak2^{V617F}-deleted) treated LSKs (Lineage−Sca1+cKit+) in comparison to MPN control LSKs isolated 7 days following initiation of TAM (n=2-3 biological replicates per arm); differentially expressed genes as indicated. FIG. 4B shows a gene set enrichment analysis (GSEA) depicting a positive enrichment in heme metabolism in ruxolitinib (RUX) treated (n=3) vs. negative enrichment in tamoxifen (TAM; Jak2^{V617F}-deleted) treated (n=3) LSKs isolated 7 days following initiation of TAM. FIG. 4C shows a row normalized heatmap of RNA-sequencing data of ruxolitinib (RUX) treated or tamoxifen (TAM; Jak2^{V617F}-deleted) treated megakaryocytic-erythroid progenitor (MEP; Lineage-cKit+Sca1−Cd34−Fcg−) cells in comparison to CTRL (MPN) cohorts. FIG. 4D shows a box plot depicting z-scored NFkB (left) or REL (right) DNA accessibility as measured by single-cell ATAC-Sequencing. FIG. 4E shows peripheral blood counts of vehicle (VEH), ruxolitinib (RUX), the type II JAK inhibitor CHZ868 (CHZ), or tamoxifen (TAM; Jak2^{V617F}-deleted) treated mice at the conclusion of a 6-week in vivo trial: white blood cells (WBC; left panel), hematocrit (Hct; right panel) (n≥4 each; mean±s.e.m.). **p<0.01, ***p<0.001, ****p<0.0001. FIG. 4F shows peripheral blood (PB) mutant CD45.2 percent chimerism trend of vehicle (VEH) vs. ruxolitinib (RUX) vs. CHZ868 (CHZ) vs. tamoxifen (TAM; Jak2^{V617F}-deleted) treated mice (n≥4 each; mean±s.e.m.). *p<0.05. FIG. 4G shows bone marrow mutant cell fraction of LSK (Lineage−Sca1+cKit+), granulocytic-monocytic progenitor (GMP; Lineage−cKit+Sca1−Cd34+Fcg+), and compartments of vehicle (VEH) vs. ruxolitinib (RUX) vs. CHZ868 (CHZ) vs. tamoxifen (TAM; Jak2^{V617F}-deleted) treated mice at the conclusion of the 6-week in vivo trial (n≥4 each; mean±s.e.m). *p<0.05, ****p<0.0001. FIG. 4H shows a GSEA depicting a negative enrichment in down regulation of KRAS signaling targets in ruxolitinib (RUX) treated (n=3) vs. positive enrichment in tamoxifen (TAM; Jak2^{V617F}-deleted) treated (n=3) MEPs isolated following 7 days of respective treatment. FIG. 4I shows immunohistochemistry of phospho-ERK on sectioned bone marrow of vehicle vs. ruxolitinib vs. tamoxifen (Jak2^{V617F}-deleted) treated mice following 7 days of treatment (n=3 individual biological replicates per arm). All images represented at 400× magnification. Scale bar: 20 m. FIG. 4J shows a quantitative polymerase-chain reaction demonstrating relative Ybx1 expression levels from isolated cKit+bone marrow of vehicle (VEH) vs. ruxolitinib (RUX) vs. tamoxifen (TAM; Jak2^{V617F}-deleted) treated mice following 7 days of treatment (n=2-4 individual biological replicates per arm; mean±s.e.m). *p<0.05, **p<0.01. e-g. Representative of n=3 independent experiments.

FIGS. 5A-5I show Jak2^{V617F} dependency with cooperative Tet2^{f/f} loss. FIG. 5A shows a schematic of the experimental set up for the double-mutant Jak2^{RL}Tet2^{f/f} competitive transplants. TAM: tamoxifen; KI: knock-in; KO: knock-out; Lin-neg BM: lineage negative bone marrow; cGy: centigray. Downward arrows represent initial pulse TAM administration to genetically inactivate Tet2. FIGS. 5B-5C show white blood cell (WBC) counts (FIG. 5B) and spleen weights (FIG. 5C) of primary Jak2^{RL} vs. Jak2^{RL}/Tet2^{f/f} transplanted mice at 12 weeks post-transplant (n=5-7 biological replicates per arm across 2 independent transplants; mean±s.d.). **p<0.01. FIG. 5D shows peripheral blood CD45.2 mutant percent chimerism of Jak2^{RL} vs. Jak2^{RL}/Tet2^{f/f} secondary competitive transplant cohorts at 12 weeks post-transplant (n=7-8 each; representative of n=2 independent transplants; mean±s.d.). **p<0.01. FIG. 5E shows peripheral blood count trends (weeks 0-21) of MPN (CTRL) vs. tamoxifen (TAM; Jak2^{V617F}-deleted) treated Jak2^{RL} vs. Jak2^{RL}/Tet2^{f/f} competitive transplant mice: white blood cells (WBC; left panel), hematocrit (Hct; right panel) (n≥4 each; mean±s.e.m). Gray bars represent duration of TAM pulse/chow administration. Representative of n=2 independent transplants. **p<0.01, ***p<0.001, ****p<0.0001. FIG. 5F shows percent change in CD45.2 mutant peripheral blood chimerism pre- vs. post-tamoxifen (TAM; Jak2^{V617F}-deletion) treatment of Jak2^{RL} vs. Jak2^{RL}Tet2^{f/f} mice in relation to CD45.1 competitor cells (n≥5 biological replicates per arm across 2 independent transplants; mean±s.e.m.). FIG. 5G shows reticulin stains of bone marrow from control (MPN) vs. tamoxifen (Jak2^{V617F}-deleted) treated Jak2^{RL} vs. Jak2^{RL}/Tet2^{f/f} mice at timed sacrifice 21 weeks. Representative micrographs of n=5 individual mouse replicates per arm. All images represented at 400× magnification. Scale bar: 20 μm. FIG. 5H shows bone marrow mutant CD45.2 percent chimerism within the LSK (Lineage−Sca1+cKit+) compartment of MPN (CTRL) vs. tamoxifen (TAM; Jak2^{V617F}-deleted) treated Jak2^{RL} vs. Jak2^{RL}/Tet2^{f/f} mice at timed sacrifice 21 weeks (n 7 biological replicates per arm across 2 independent transplants; mean±s.e.m). *p<0.05, ***p<0.001. FIG. 5I shows a serial replating assay of plated MPN (CTRL) vs. tamoxifen (TAM; Jak2^{V617F}-deleted) treated Jak2^{RL} VS. Jak2^{RL}/Tet2^{f/f} bone marrow cells harvested at timed sacrifice 21 weeks and scored at day 8 after each plating (each sample plated in triplicate, representative of n=2 independent experiments, mean±s.d.).

FIGS. 6A-6H show phenotypic characterization and validation of Jak2^{RL} activation in vivo. FIG. 6A shows Sanger sequencing of the Jak2^{RL} locus (SEQ ID NO: 82) from genomic DNA of sorted Cre TdTomato+ reporter bone marrow cells following tamoxifen administration (“Jak2^{RL}+Cre”) compared to the non-recombined Jak2^{RL} locus (Jak2^{RL}) demonstrating retainment of Lox/Rox sites despite prior Cre exposure. Representative of n=3 individual biological replicates. FIG. 6B shows a schematic of the Dre mRNA Jak2^{RL} bone marrow electroporation/transplant protocol. Lin-neg BM: lineage-negative bone marrow; cGy: centigray. FIG. 6C shows a representative polymerase chain reaction (PCR) of genomic DNA isolated from peripheral blood mononuclear cells in primary transplant recipients following Dre mRNA electroporation (+Dre) confirming the presence of Jak2^{V617F} knock-in bands (“Jak2^{RL} KI”) in relation to the Jak2 wild-type (“Jak2 WT”) allele in comparison to unrecombined (−Dre) Jak2^{RL} cells. FIG. 6D shows peripheral blood counts of wild-type (WT) vs. Jak2^{RL} knock-in (Jak2^{RL}) mice in comparison to the previously-published Cre-lox conditional Jak2^{V617F} knock-in mouse model (Jak2Crelox)8 at timed sacrifice of 16 weeks: white blood cells (WBC; left panel), hematocrit (Hct; middle panel), platelets (Plt; right panel) (n=5 each; mean±s.e.m). *p<0.05, **p<0.01, ****p<0.0001. FIG. 6E shows Ter 19+CD71+ erythroid progenitor fractions (left panel) and representative fluorescence-activated cell sorting (FACS) plots (right panel) from harvested spleen of Jak2^{RL} knock-in (Jak2^{RL}) mice in comparison to wild-type (WT) and the previously-published Jak2Crelox mouse model (n=5 each; mean±s.e.m). Black boxes on FACS plots denote Ter 19+ erythroid progenitor stages I-IV. **p<0.01. FIG. 6F shows bone marrow myeloid progenitor (Lineage−cKit+Sca1-) frequencies of Jak2^{RL} knock-in (Jak2^{RL}) mice in comparison to wild-type (WT) and the previously-published Jak2Crelox mouse model at timed sacrifice 16 weeks post-transplant (n=5 each; mean±s.e.m.). *p<0.05, **p<0.01. FIG. 6G shows spleen weights (left panel) and representative spleens (right panel) of Jak2^{RL} knock-in (Jak2^{RL}) mice in comparison to wild-type (WT) and the previously-published Jak2Crelox mouse model at timed sacrifice 16 weeks post-transplant. **p<0.01, ***p<0.001. FIG. 6H shows hematoxylin and eosin (H&E) stains of bone marrow and spleen of Jak2^{RL} knock-in (Jak2^{RL}) mice in comparison to wild-type (WT) controls at timed sacrifice 16 weeks post-transplant. Representative micrographs of n=5 individual mouse replicates per arm. Bone marrow images represented at 400× magnification (scale bar 20 μm); spleen images 100× (scale bar 100 μm). FIGS. 6D-6H are representative of an n=3 independent transplants.

FIGS. 7A-7I show functional consequences of Jak2^{V617F} reversion ex vivo. FIG. 7A shows a Schematic of the bone marrow endothelial cell (BMEC) co-culture assay. Lin-neg BM: lineage-negative BM; EtOH: ethanol vehicle; 40HT: 4-hydroxy-tamoxifen; FACS: fluorescence-activated cell sorting. FIG. 7B shows a representative Jak2^{V617F} knock-in (+Dre) and knock-out (+Dre+Cre) genotyping by polymerase chain reaction (PCR) of genomic DNA isolated from cultured bone marrow cells following Dre-mediated knock-in (+Dre) and/or subsequent Cre-mediated deletion (+Dre+Cre) in comparison to the unrecombined Jak2^{RL} allele (CTRL). Representative bands of n=4 individual replicates per arm. FIG. 7C shows total cell output of cultured wild-type (data points at middle) vs. Jak2^{RL} knock-in (data points at bottom) cells in comparison to Cre-inducible Jak2^{V617F} knock-in (Jak2Crelox; data points at top) cells following 7 days of culture over BMECs in the presence of increasing doses (0 nM-1000 nM) of 4-hydroxy-tamoxifen (40HT) (n=3 technical replicates each; mean±s.d.). Representative of n=3 independent experiments. ns: not significant, **p<0.01, ****p<0.0001. FIGS. 7D-7G show total cell (FIG. 7D), Mac1+Gr1+ mature neutrophil (FIG. 7E), Myeloid progenitor (Lineage−cKit+Sca1−) (FIG. 7F), and Megakaryocytic-erythroid progenitor (MEP; Lineage−cKit+Sca1−Cd34−Fcg−) (FIG. 7G) cell output of Jak2^{RL} knock-in cells in comparison to wild-type vs. Cre-inducible Jak2Crelox MPN cells following 7 days of culture over BMECs in the presence of vehicle (VEH) vs. 400 nM 4-hydroxy-tamoxifen (40HT) (n=3 technical replicates each; mean±s.d.). Representative of n=3 independent experiments. ***p<0.001, ****p<0.0001. FIG. 7H shows total erythroid progenitor cell numbers by Ter 19/CD71 erythroid progenitor (I-IV; left panel) flow cytometry and representative Ter 19/CD71 FACS plots (right panel) of cultured Jak2^{RL} MPN cells following 7 days of culture over BMECs in the presence of vehicle (VEH) vs. 400 nM 4-hydroxy-tamoxifen (40HT) (n=3 technical replicates each; mean±s.d.). Representative of n=2 independent experiments. FIG. 7I shows percentages of Mac1+Gr1+ apoptotic cells of Jak2^{RL} cells in comparison to wild-type vs. Jak2Crelox cells following 7 days of culture over BMECs in the presence of vehicle (VEH) vs. 200 nM 4-hydroxy-tamoxifen (40HT) (n=3 technical replicates each; mean±s.d.). Representative of n=2 independent experiments. *p<0.05, **p<0.01.

FIGS. 8A-8F show functional characterization of JAK2^{RL} knock-in/knock-out in vivo. FIG. 8A shows a schematic of the experimental set up for the Jak2^{RL} knock-in/knock-out transplants. BM: bone marrow; TAM: tamoxifen; cGy: centigray. FIG. 8B shows a representative polymerase chain reaction (PCR) demonstrating presence or loss of Jak2^{RL} knock-in bands (“Jak2^{RL} KI”) in relation to the Jak2 wild-type (“Jak2 WT”) allele from reporter-sorted whole bone marrow mononuclear cells based on different recombined states: Pre-Dre: unrecombined Jak2^{RL} cells; double-neg: reporter-negative cells following Dre recombination; TdTomato+: Jak2^{V617F} knock-in population following Dre recombination; GFP+: green fluorescent protein (GFP) population: Jak2^{V617F}-deleted population (i.e. Dre followed by Cre). FIG. 8C shows Jak2^{V617F} mutant RNA transcript variant allele frequency (VAF) from isolated megakaryocytic-erythroid progenitor (MEP; Lineage−cKit+Sca1−Cd34−Fcg−) cells 7 days following tamoxifen (TAM; Jak2^{V617F} deletion) treatment in comparison to MPN (CTRL) mice (n=3-5 each; mean±s.d.). ****p<0.0001. FIG. 8D shows platelet (Plt) counts of MPN (CTRL) vs. tamoxifen (TAM; Jak2^{V617F}-deleted) treated mice at timed sacrifice 24 weeks (n≥10 each; mean±s.e.m). Representative of n=2 independent transplants. ***p<0.001. FIG. 8E shows a representative polymerase chain reaction (PCR) demonstrating presence of Jak2^{V617F} knock-in (KI) bands in relation to the Jak2 wild-type (WT) allele from isolated bone marrow mononuclear cells of transplant recipients previously treated with tamoxifen (Jak2^{V617F} deletion) and exhibiting phenotypic features of recurrent MPN (n=2) in comparison to those with no recurrent MPN (representative of n=8 biological replicates). FIG. 8F shows hematoxylin and eosin (H&E) and Ter 19 immunohistochemistry of sectioned spleen of MPN (Control) vs. tamoxifen treated (Jak2^{V617F}-deleted) mice at timed sacrifice 24 weeks. Representative micrographs of n=7 individual mouse replicates. All images represented at 100× magnification. Scale bar: 100 μm.

FIGS. 9A-9G show assessment of tamoxifen toxicity on JAK2^{RL} knock-in cells. FIG. 9A shows peripheral blood count trend of MPN (CTRL) vs. tamoxifen (TAM) treated mice transplanted with Cre-negative JAK2^{RL} knock-in cells in competition with CD45.1 support (weeks 0-15): white blood cells (WBC; left panel), hematocrit (Hct; middle panel), platelets (PLT; right panel) (n=3-4 each; mean±s.e.m); Gray bars represent duration of TAM pulse/chow administration; ns=not significant, *p<0.05. FIG. 9B shows peripheral blood (PB) mutant CD45.2 percent chimerism trend (weeks 0-15) of MPN (CTRL) vs. tamoxifen (TAM) treated mice transplanted with Cre-negative JAK2^{RL} knock-in cells in competition with CD45.1 support (n=3-4 each; mean±s.e.m); Gray bar represents duration of TAM pulse/chow administration. FIG. 9C shows spleen weights (left panel) and representative isolated spleens (right panel) of MPN (CTRL) vs. tamoxifen (TAM) treated mice transplanted with Cre-negative JAK2^{RL} knock-in cells in competition with CD45.1 support at timed sacrifice 15 weeks (n=3-4 each; mean±s.e.m). FIGS. 9D-9E show total bone marrow (BM) cellularity (femur) (FIG. 9D) and Myeloid progenitor (Lineage−cKit+Sca1−) (FIG. 9E) frequencies of MPN (CTRL) vs. tamoxifen (TAM) treated mice transplanted with Cre-negative JAK2^{RL} knock-in cells in competition with CD45.1 support at timed sacrifice 15 weeks (n=3-4 each; mean±s.e.m). FIG. 9F shows bone marrow mutant cell fraction of LSK (Lineage−Sca1+cKit+), granulocytic-monocytic progenitor (GMP; Lineage−cKit+Sca1-Cd34+Fcg+), and megakaryocytic-erythroid progenitor (MEP; Lineage−cKit+Sca1−Cd34−Fcg−) compartments of MPN (CTRL) vs. tamoxifen (TAM) treated mice transplanted with Cre-negative JAK2^{RL} knock-in cells in competition with CD45.1 support at timed sacrifice 15 weeks (n=3-4 each; mean±s.e.m.). FIG. 9G shows hematoxylin and eosin (H&E) stains of bone marrow (BM) of MPN (CTRL) vs. tamoxifen (TAM) mice transplanted with Cre-negative JAK2^{RL} knock-in cells in competition with CD45.1 support at timed sacrifice 15 weeks; Representative micrographs of n=3-4 individual mouse replicates; Images represented at 400× magnification; Scale bar: 20 μm. FIGS. 9A-9G are representative of n=3 independent experiments.

FIGS. 10A-10H show effects of Jak2^{V617F} deletion on MPN stem cell fitness and transplantability. FIG. 10A shows a schematic of the experimental set up for the Jak2^{RL} competitive transplant studies. TAM: tamoxifen; cGy: centigray. FIG. 10B shows peripheral blood Mac1+Gr1+ mutant CD45.2 percent chimerism trend (weeks 0-24) of “early” (3-weeks post-transplant) tamoxifen (TAM; Jak2^{V617F}-deleted) treated (data points at bottom) and “late” (12-weeks posttransplant) tamoxifen treated (data points at middle) mice (n=7-9 each) in comparison to MPN (CTRL; data points at top; n=7) mice (mean±s.e.m.). Gray bars represent duration of TAM pulse/chow administration. Representative of n=2 independent transplants. **p<0.01, ***p<0.001. FIG. 10C shows total myeloid progenitor (MP; Lineage-cKit+Sca1−) numbers of “early”-treated and “late”-treated Jak2^{V617F} knock-in vs. MPN (CTRL) mice at timed sacrifice 24 weeks (n=7-9 each; mean±s.e.m.). *p<0.05. FIG. 10D shows representative fluorescence-activated cell sorting (FACS) plots demonstrating mutant CD45.2 to competitor CD45.1 percentage of gated LSK (Lineage−Sca1+cKit+) cells of MPN (CTRL) vs. tamoxifen (TAM; Jak2^{V617F}-deleted) treated mice at timed sacrifice 24 weeks (representative of n≥6 biological replicates per arm). FIG. 10E shows bone marrow mutant CD45.2 percentage within the long-term hematopoietic stem cell (LT-HSC; Lineage-Sca1+cKit+CD150⁺CD48−) compartment of “early” (3-weeks post-transplant) tamoxifen (TAM; Jak2^{V617F}-deleted) treated and “late” (12-weeks post-transplant) tamoxifen treated mice in comparison to MPN (CTRL) mice at timed sacrifice 24 weeks (n=6-8 individual biological replicates per arm; mean±s.e.m.). Representative of n=2 independent transplants. **p<0.01, ****p<0.0001. FIGS. 10F-10G show hematocrit (Hct) (FIG. 10F) and peripheral blood mutant CD45.2 fraction (FIG. 10G) of mice transplanted with MPN (CTRL) vs. tamoxifen (TAM; Jak2^{V617F}-deleted) treated donor bone marrow cells at 12 weeks post-transplant (n=5 each; mean±s.e.m). Representative of n=2 independent experiments. *p<0.05, **p<0.01. FIG. 10H shows a polymerase chain reaction (PCR) demonstrating presence of a Jak2^{V617F} knock-in (KI) band in relation to the Jak2 wild-type (WT) allele in 1 of 5 recipients (asterisk) transplanted with TAM-treated (Jak2^{V617F}-deleted) donor cells demonstrating recurrence of MPN phenotype in comparison to donor mice transplanted with MPN (CTRL) bone marrow donor cells.

FIGS. 11A-11G show acute phenotypic and transcriptional changes following Jak2^{V617F} reversion. FIG. 11A shows a heatmap (left panel) and gene set enrichment analysis (GSEA; right panel) demonstrating negative enrichment of STAT5 targets, including negative regulators of JAK/STAT signaling, in LSK (Lineage−Sca1+cKit+) cells harvested 3 days (D3)+/−7 days (D7) following initiation of tamoxifen (Jak2^{RL}-deletion) treatment in comparison to MPN (CTRL) cells. FIG. 11B shows a GSEA demonstrating negative enrichment in KEGG:MAPK signaling targets from harvested LSKs 7 days following initiation of tamoxifen treatment in comparison to MPN control (CTRL) LSKs. FIG. 11C shows Mac1+ immunohistochemistry (IHC) of sectioned bone marrow (left panel) and bone marrow Mac1+Gr1+ frequencies by fluorescence-activated cell sorting (right panel) of MPN (CTRL) vs. tamoxifen (TAM; Jak2^{RL}-deleted) treated mice at 7 days following tamoxifen administration (n=4 each; mean±s.e.m.). Representative micrographs of n=4 individual mouse replicates. Images represented at 400× magnification. Scale bar: 20 μm. **p<0.01.

FIGS. 11D-11E show total erythroid progenitor bone marrow cell numbers (FIG. 11D), and Representative fluorescence-activated cell sorting (FACS) plots (FIG. 11E) of Ter119/CD71 erythroid progenitor populations of MPN (CTRL) vs. tamoxifen (TAM; Jak2^{RL}-deleted) treated mice 7 days following TAM administration (n=4-7/arm; mean±s.e.m.). *p<0.05, **p<0.01, ***p<0.001. FIG. 11F shows burst-forming unit erythroid (BFU-E) colony output of MPN (CTRL) vs. tamoxifen (TAM; Jak2^{RL}-deleted) treated marrow isolated 7 days following TAM administration and scored at day 8 after plating (each sample plated in triplicate, mean±s.e.m.). FIG. 11G shows normalized ATAC-Seq signal at the EpoR locus (top panel) with associated quantified normalized peak counts (bottom panel) demonstrating decreased accessibility in tamoxifen (TAM; Jak2^{RL}-deleted) treated cKit+ bone marrow cells 7 days following initiation of TAM in comparison to MPN (CTRL) cells. FIGS. 11C-11F are representative of n≥2 independent experiments.

FIGS. 12A-12G show differential responses of Jak2^{V617F} deletion compared to JAK inhibitor therapy in vivo. FIG. 12A shows normalized read counts of representative JAK/STAT targets from LSK (Lineage−Sca1+cKit+) cells isolated 7 days following ruxolitinib (RUX) vs. tamoxifen (TAM; Jak2^{V617F}-deletion) treatment compared to vehicle (Veh) controls (n=2-4 per arm; mean±s.e.m.). FIG. 12B shows LSK (Lineage-Sca1+cKit+; left panel), granulocytic-monocytic progenitor (GMP; Lineage−cKit+Sca1−Cd34+Fcg+; middle panel), and megakaryocytic-erythroid progenitor (MEP; Lineage-cKit+Sca1−Cd34−Fcg−; right panel) bone marrow frequencies of tamoxifen (TAM; Jak2^{V617F}-deleted) treated mice in comparison to vehicle (VEH) or ruxolitinib (RUX) treated mice 7 days following respective treatment (n=3-5 per arm; mean±s.e.m.). Data representative of n=3 independent experiments. *p<0.05, ***p<0.001, ****p<0.0001. FIG. 12C shows hierarchical clustering demonstrating unchanged NFkB accessibility motifs in untreated vs. ruxolitinib treated Jak2^{V617F} mutant HSPCs of MF patients. FIG. 12D shows a schematic of the competitive transplant set up for the Jak2^{RL} in vivo drug studies. cGy: centigray. FIGS. 12E-12F show platelet (PLT) counts (FIG. 12E) and spleen weights (FIG. 12F) of vehicle (VEH) vs. ruxolitinib (RUX) vs. CHZ868 (CHZ) vs. tamoxifen (TAM; Jak2^{V617F}-deleted) treated mice at the conclusion of the 6-week in vivo trial (n 4 each; mean±s.e.m). *p<0.05, **p<0.01, ***p<0.001, ****p<0.0001. Representative of n=3 independent experiments. FIG. 12G shows hematoxylin and eosin (H&E) stains of bone marrow and spleen sections from vehicle vs. ruxolitinib vs. tamoxifen (Jak2^{V617F}-deleted) treated mice at the conclusion of the 6-week in vivo trial (n 4 each; mean±s.e.m). Representative micrographs of n=4 individual mouse replicates per arm. Bone marrow images represented at 400× magnification (scale bar 20 μm); spleen images 100× (scale bar 100 μm).

FIGS. 13A-13G show assessment of Jak2^{V617F} oncogenic dependency in the setting of concomitant Tet2 loss. FIG. 13A shows hematocrit (Hct; left panel) and platelet counts (Plt; right panel) of Jak2^{RL} vs. Jak2^{RL}Tet2^{f/f} transplanted mice at 12-weeks post-transplant (n≥5 biological replicates per arm across 2 independent transplants; mean±s.d.). FIG. 13B shows a serial replating assay of Jak2^{RL} vs. Jak2^{RL}/Tet2^{f/f} bone marrow cells in relation to wild-type (WT) bone marrow. Colonies were counted at day 8 after each plating (each sample plated in triplicate, n=2 independent experiments, mean±s.d.). FIG. 13C shows total cell (left panel) and Mac1+Gr1+ mature neutrophil cell (right panel) output of Jak2^{RL} vs. Jak2^{RL}Tet2^{f/f} cells following 7 days of culture over bone marrow endothelial cells (BMECs) (n=3 replicates per arm; mean±s.d.). Representative of n=3 independent experiments. *p<0.05, **p<0.01. FIG. 13D shows spleen weights (left panel) and representative isolated spleens (right panel) of MPN (CTRL) vs. tamoxifen (TAM; Jak2^{V617F}-deleted) treated Jak2^{RL} vs. Jak2^{RL}/Tet2^{f/f} mice at timed sacrifice 21 weeks (n≥7 biological replicates per arm across 2 independent transplants; mean±s.d.). *p<0.05, ****p<0.0001. FIGS. 13E-13F show total bone marrow (BM) cellularity (femur) (FIG. 13E), and bone marrow mutant cell fraction (FIG. 13F) within the granulocytic-monocytic progenitor (GMP; Lineage−cKit+Sca1−Cd34+Fcg+; left panel) and megakaryocytic-erythroid progenitor (MEP; Lineage−cKit+Sca1−Cd34−Fcg−; right panel) compartments of MPN (CTRL) vs. tamoxifen (TAM; Jak2^{V617F}-deleted) treated Jak2^{RL} vs. Jak2^{RL}Tet2^{f/f} mice at timed sacrifice 21 weeks (n≥7 biological replicates per arm across 2 independent transplants; mean±s.e.m.). *p<0.05, **p<0.01, ***p<0.001. FIG. 13G shows Tet2^{f/f} excision genotyping by polymerase chain reaction (PCR) of genomic DNA isolated from whole bone marrow of MPN (CTRL) vs. tamoxifen (TAM; Jak2^{V617F}-deleted) treated Jak2^{RL} vs. Jak2^{RL}Tet2^{f/f} mice demonstrating presence of Tet2^{f/f} excised (knock-out; KO) bands in relation to Tet2 wild-type bands. Representative bands of n=4-7 individual replicates per arm across 2 independent transplants.

FIG. 13H shows a general gating strategy for bone marrow hematopoietic stem/progenitor cell analysis.

FIGS. 14A-14G show Flp-recombinase inducible Flt3^{ITD} activation. FIG. 14A shows a schematic Flt3^{Frt-ITD} targeted to the endogenous Flt3 locus replacing WT exons; Flp-recombination deletes WT exons and inverts mutant exons resulting in Flt3^{ITD} expression. FIG. 14B shows a strip chart of WBC K/uL in WT (left) or Rosa26:FlpoERT2 Flt3^{Frt-ITD} (right) mice at the indicated timepoint posts TAM. FIG. 14C shows H&E staining of bone marrow or spleen in WT or Rosa26:FlpoERT2 Flt3^{Frt-ITD} mice 16 weeks post TAM treatment (400×). FIG. 14D shows a strip chart indicating % lin⁻ cells in WT (Ctrl) or Flt3^{Frt-ITD} bone marrow. FIG. 14E shows a row normalized heatmap of RNA-sequencing data in WT or Flt3^{Frt-ITD} LSKs following TAM treatment at the indicated timepoints. FIG. 14F shows Z-scored ssGSEA values for the indicated Hallmark geneset in either WT or Flt3^{Frt-ITD} mice following TAM treatment. FIG. 14G shows peripheral blood chimerism (% CD45.2) in mice engrafted with WT (CD45.1) and Flt3^{Frt-ITD} (CD45.2) cells following treatment with TAM (data points at top) or control (data points at bottom).

FIGS. 15A-15H show Flt3^{ITD}-driven models of leukemogenesis. FIG. 15A shows Kaplan-Meier survival curve for Rosa26:FlpoERT2 WT (line at top), Flt3^{Frt-ITD} (line at bottom middle), Npm1^{c-Frt} (line at bottom right), or Npm1^{c-Frt} Flt3^{Frt-ITD} (line at bottom left) following treatment with TAM. FIGS. 15B-15C show strip charts showing results for experiments in which bone marrow was transplanted into lethally irradiated recipient mice; after transplant (4w) mice were treated with TAM to activate the indicated genotypes; strip chart indicates WBC (FIG. 15B) or HCT (FIG. 15C) 4 weeks post TAM. FIG. 15D shows row-normalized heatmap of RNA-sequencing data in WT, Flt3^{Frt-ITD}, Npm1^{c-Frt}, or Npm1^{c-Frt} Flt3^{Frt-ITD} LSKs following TAM treatment at the indicated timepoints. FIG. 15E shows bone marrow transplant-mediated sequential mutagenesis experimental schematic indicating order of Dnmt3a^{Loox-R878H} (Cre mRNA) and Flt3^{Frt-ITD} (TAM) activation. FIG. 15F shows results from experiments in which bone marrow from CD45.1 WT or CD45.2 cells from Dnmt3a^{Loox-R878H}; Flt3Frt-ITD or Flt3^{Frt-ITD} only mutant mice >20w after TAM treatment were transplanted in competition into secondary lethally irradiated recipients; peripheral blood chimerism (% CD45.2) depicted for individual mice. FIG. 15G shows normalized ssGSEA scores on RNA-sequencing data from LSKs sorted from symptomatic Npm1^{c-Frt} Flt3^{Frt-ITD} or Dnmt3a^{Loox-R878H} Flt3^{Frt-ITD} mice; genesets were derived from FIG. 21C. FIG. 15H shows a boxplot depicting % bone marrow cells with a Granulocyte-like (left, Cluster 1) or MDP-like (right, Cluster 5) immunophenotype characterized by CyTOF.

FIGS. 16A-16B show dual-recombinase, reversible models of oncogene activation. FIG. 16A shows a schematic depicting GOLDI-Lox knockin Flt3^{GL-ITD} construct to the endogenous Flt3 locus, replacing exons 13-15 (flanking exons 12 and 16 are indicated by box shapes and labeled, Lox2272 sites are indicated by triangles, heterotypic RoxP and Rox12 sites are indicated by chevrons and labeled); Inverted exons 13-15 are present at baseline with the W51 ITD encoded in exon 14; A′, B′ and C′ indicate relative position of primers used in FIG. 16B to detect Dre-mediated inversion and Cre-mediated deletion. FIG. 16B shows results from experiments in which Lin bone marrow from WT of Ubc:CreERT2 Flt3^{GL-ITD} mice was infected with MSCV:Dre-IRES-GFP retrovirus or mock infection; Infected cells were treated+/−4-OHT to activate Cre, and DNA was isolated from the whole culture 72 hours post treatment; PCR was used to evaluate Dre-inversion and Cre-deletion, visualized by capillary gel electrophoresis (Qiaxcel).

FIGS. 17A-17F show triple-mutant, sequential models of clonal evolution towards acute myeloid leukemia. FIG. 17A shows a schematic depicting sequential mutagenesis scheme with Dnmt3a^{Los-R878H} being activated at first transplant with Cre mRNA, Npm1^{c-Frt} activated post-transplant with TAM-mediated Rosa26:FlpoERT2 activation, and Flt3^{GL-ITD} finally activated with Dre mRNA at secondary transplant. FIG. 17B shows peripheral blood monocyte count (K/μL) in secondary transplant for the indicated genotypes. FIG. 17C shows a heatmap depicting relative abundance of indicated cell populations (rows) for the indicated genotypes (columns) in bone marrow isolated from mice either 20 weeks post-transplant (WT, Dnmt3a^{Los-R878H}, Npm1^{c-Frt}, Dnmt3a^{Los-R878H}Npm1^{c-Frt}) or when symptomatic (Npm1^{c-Frt} 2°, Dnmt3a^{Los-R878H}Npm1^{c-Frt} 2°, Npm1^{c-Frt} Flt3^{GL-ITD-}, or Dnmt3a^{Los-R878H} Npm1^{c-Frt} Flt3^{GL-ITD-}); Secondary transplant indicated by 2°, following either Dre-mRNA or mock electroporation. FIGS. 17D-17F show strip charts from mice depicted in FIG. 17C with y-axis indicated % of CD45.2 in bone marrow from either LT-HSCs (FIG. 17D), Cd11b+Gr1+ cells (FIG. 17E) or LSKs (FIG. 17F).

FIGS. 18A-18E show chemically-inducible orthogonal recombinase activation. FIG. 18A shows a schematic depicting DreSTAPL-ODC construct indicating that treatment with NS3 protease inhibitor results in complex formation of the split N and C-terminus of Dre; in the absence of NS3-inhibitin, proteolytic cleavage reveals the ODC degron leading to degradation of the N-terminus. FIG. 18B shows results from experiments in which Lin⁻ bone marrow from Rosa26: TdTomato (Lox-stop-Lox, Ai14), Rosa26:RLTG (Rox-stop-rox) or Rosa26:FLTG (Frt-stop-Frt) mice were infected with retroviral vectors encoding GFP and CreER, DreSTAPL-ODC, or FlpoDHFR, respectively; Cultures were treated with the DMSO, 4-OHT (400 nM), GZV (10 μM) or TMP (1 μM) as indicated; Barplot depicts recombination in infected population (% TdTomato+ of GFP+ cells). FIG. 18C shows experimental schema for primary mice with Ubc:CreERT2, Vav1:DreSTAPL-ODC, Flt3^{GL-ITD} as well as either Idh2^{Lox-R140Q} or Npm1^{Lox-C}; Schema depicts dosing schedule of either TAM or GZV to activate Cre and Dre, respectively. FIG. 18D shows a line chart depicting in peripheral blood monocyte (K/IL) counts in Flt3^{GL-ITD}, Npm1^{Lox-C} Flt3^{GL-ITD} or Idh2^{Lox-R140Q} Flt3^{GL-ITD} mice following GZV treatment. FIG. 18E shows a stripchart indicating % Cd11b+ cells in the peripheral blood of mice depicted in FIGS. 18C-18D at either 16 weeks post GZV treatment (Flt3^{GL-ITD} and Idh2^{Lox-R140Q} Flt3^{GL-ITD}) or when symptomatic (Npm1^{Lox-C} Flt3^{GL-ITD})

FIGS. 19A-19H show reversible Flt3 mutagenesis in acute myeloid leukemia. Secondary transplant of Npm1^{Lox-C} Flt3^{GL-ITD} (20,000 cells) or Idh2^{Lox-R140Q} Flt3^{GL-ITD} (500,000) from FIGS. 18A-18E were engrafted into lethally irradiated secondary recipients with 500,000 CD45.1 WT cells. After transplant, mice were monitored for disease development (Npm1^{Lox-C} Flt3^{GL-ITD} 4 weeks, or Idh2^{Lox-R140Q} Flt3^{GL-ITD} 10 weeks), and then euthanized following 7 days of TAM treatment (FIGS. 19A-19G). FIG. 19A shows peripheral blood WBC (K/uL) before and after TAM treatment. FIG. 19B shows spleen mass in control and TAM treated mice. FIG. 19C shows bone marrow H&E stained from either control mice or after 7 days of TAM (400×). FIG. 19D shows a dot chart depicting % LSKs of Cd45.2 cells in bone marrow from control and TAM treated mice for the indicated disease models. FIG. 19E shows RNA-sequencing data from lin⁻ HSPCs purified from 7-day TAM treated Npm1^{Lox-C} Flt3^{GL-ITD} or control mice; Gene ranks from GSEA are depicted as a line chart for the indicated HALLMARK gene set. FIG. 19F shows serum cytokine levels for IL6 (left) and G-CSF (right) from WT, untreated Npm1^{Lox-C} Flt3^{GL-ITD}, and TAM-treated (7d) Npm1^{Lox-C} Flt3^{GL-ITD} mice. FIG. 19G shows immunohistochemical staining on bone marrow for Ki67 (left) and phopsho-ERK1/2 (right) for untreated (top) and 7-day TAM treated (bottom) Npm1^{Lox-C} Flt3^{GL-ITD} mice. FIG. 19H shows Kaplan-Meier survival curve of mice engrafted with 500,000 CD45.1 WT cells and 40,000 bone marrow cells from symptomatic Npm1^{Lox-C} Flt3^{GL-ITD} mice from FIGS. 18A-18E; Mice were either treated with control chow (grey) or placed on a TAM-chow diet.

FIGS. 20A-20G show Flp-inducible Flt3^{Frt-ITD} mice demonstrate aberrant hematopoietic phenotypes. FIG. 20A shows results from experiments in which Lin⁻ bone marrow was isolated from mice encoding a transcriptional stop cassette flanked by either Lox, Rox or Frt sites; Cells were infected with an GFP+ retrovirus encoding either Cre, Dre or Flp; Excision of the stop cassette in all three reporter lines results in TdTomato expression; Histogram depicts TdTomato expression in GFP+, virally infected cells. FIG. 20B shows results from experiments in which Rosa26:FlpoERT2 Flt3^{Frt-ITD} or WT mice were treated with TAM, and DNA was isolated for PCR to evaluate excision at the Flt3 locus; Lower band indicates WT Flt3, upper band indicates Flp-mediated inversion; PCR was visualized with capillary electrophoresis (Qiaxcel). FIG. 20C shows results from experiments in which lethally irradiated Cd45.1 recipient mice were transplanted with Rosa26:FlpoERT2 Flt3^{Frt-ITD} bone marrow and treated with TAM 4 weeks after engraftment; Boxplot depicting total myeloid (Cd11b⁺, left) and cKIT+ (right) cells of Cd45.2 cells in bone marrow transplant at 2, 4, 6 and 8 weeks post TAM; Control mice are Flt3w and 4 weeks post TAM. FIG. 20D shows hematocrit (%, left), platelets (K/μL, middle) and monocytes (K/μL, right) from primary Rosa26:FlpoERT2 Flt3^{Frt-ITD} or WT at 2, 4, 6, and 8 weeks post TAM. FIG. 20E shows results from experiments in which, as in FIG. 20C, mice were transplanted with Rosa26:FlpoERT2 Flt3^{Frt-ITD} bone marrow and treated with TAM 4 weeks after engraftment; Boxplots depict spleen mass 2, 4, 6 and 8 weeks post TAM; Control mice are Flt3w and 4 weeks post TAM. FIG. 20F shows H&E staining of bone marrow from mice described in FIGS. 20C and 20E (400×). FIG. 20G shows a dot chart depicting % LSK of Cd45.2 cells in bone marrow (left), spleen (middle), or % Lin⁻ of Cd45.2 in spleen (right) from mice transplanted with Rosa26:FlpoERT2 Flt3^{Frt-ITD} at 2, 4, 6, and 8 weeks post TAM.

FIGS. 21A-21I show transcriptional alterations associated with stem cell dysregulation in Flt3^{Frt-ITD} mice. FIG. 21A shows a principal component analysis on RNA-sequencing data of the 1,000 most variable genes across LSKs and GMPs sorted from WT or Flt3^{Frt-ITD} LSKs at either 2, 4, 6 or 8 weeks post TAM. FIG. 21B shows a barplot depicting number of downregulated and upregulated differentially expressed genes in LSKs for the indicated comparisons. FIG. 21C shows a row-normalized heatmap depicting average expression levels for gene clusters identified by K-means clustering. FIG. 21D shows a boxplot depicting normalized gene expression values in WT or Flt3^{Frt-ITD} LSKs at either 2, 4, 6 or 8 weeks post TAM. FIG. 21E shows a row-normalized heatmap of RNA-sequencing data in WT or Flt3^{Frt-ITD} LSKs and GMPs at either 2, 4, 6 or 8 weeks post TAM. FIG. 21F shows a boxplot depicting normalized gene expression values for stem related genes in WT or Flt3^{Frt-ITD} LSKs as in FIG. 21D. FIG. 21G shows results from experiments in which Primary Rosa26:FlpoERT2 Flt3^{Frt-ITD} mice were treated with TAM at 6 weeks of age, and bone marrow was harvested 24 weeks after TAM; Cells (2×10⁶) from Flt3^{Frt-ITD} mice were mixed with Cd45.1 whole bone marrow (3×10⁵) and transplanted into lethally irradiated Cd45.1 recipients; Line chart depicts Cd45.2 chimerism in peripheral blood. FIG. 21H shows results from experiments in which Rosa26:FlpoERT2 Flt3^{Frt-ITD} was mixed with Cd45.1 WT cells 1:1 and transplanted into lethally irradiated Cd45.1 recipient mice; Mice were treated with TAM 4 weeks post engraftment, and euthanized 16 weeks post TAM; Barplot depicts Cd45.2 chimerism at 16 weeks post TAM. FIG. 21I shows results from experiments in which, as in FIG. 21H, whole bone marrow was isolated from mice 16 weeks post TAM, and LT-HSC (lin⁻ cKIT⁺ Sca1⁺ Cd48⁻ Cd150⁺) were quantified by flow cytometry in both the Cd45.1 WT and Cd45.2 mutant compartment.

FIGS. 22A-22H show Npm1^{Frt-C} and Flt3^{Frt-ITD} synergize to form an aggressive leukemia. FIG. 22A shows a schematic indicating simultaneous activation of Npm1^{Frt-c} and Flt3^{Frt-ITD} by TAM-mediated FlpoERT2 activation. FIG. 22B shows results from experiments in which, as in FIG. 21B, bone marrow was transplanted into lethally irradiated recipient mice; After transplant (4w) mice were treated with TAM to activate the indicated genotypes; Stripchart indicates peripheral blood platelets (K/μL) 4 weeks post TAM. FIG. 22C shows H&E staining on bone marrow from the indicated genotypes 4 weeks post TAM as in FIG. 22B. FIG. 22D shows results from experiments in which bone marrow from Rosa26:FlpoERT2 Npm1^{Frt-C} Flt3^{F}rt-ITD mice were transplanted into lethally irradiated recipients and treated with the indicated titration of TAM; Boxplots depict WBC (K/μL) at biweekly bleeds post TAM. FIG. 22E shows a principal component analysis of the 1,000 most variable genes across LSKs and GMPs sorted from WT, Npm1^{Frt-C}, Flt3^{Frt-ITD} and Npm1^{Frt-C} Flt3^{Frt-ITD} mice 4 weeks after TAM. FIG. 22F shows a barplot depicting number of downregulated and upregulated differentially expressed genes for the indicated comparison. FIGS. 22G-22H show results from GSEA depicting an enrichment of KEGG: MAPK Signaling (FIG. 22G) and Hallmark: TNFalpha signaling via NFKB (FIG. 22H) in Npm1^{Frt-c} Flt3^{Frt-ITD} vs Flt3^{Frt-ITD} cells.

FIGS. 23A-23E show disease kinetics and transcriptional characterization of Dnmt3a-Flt3 and Npm1-Flt3 leukemias. FIG. 23A shows a line chart depicting monocytes (K/μL) of WT, Dnmt3a^{Lox-R878H}, Flt3^{Frt-ITD} and Dnmt3a^{Lox-R878H} Flt3^{Frt-ITD} mice following TAM-mediated activation of Rosa26:FlpoERT2. FIG. 23B shows Kaplan-Meier survival curve of Flt3^{Frt-ITD}, Dnmt3a^{Lox-R878H} Flt3^{Frt-ITD}, and Npm1^{Lox-C} Flt3^{Frt-ITD} mice following TAM-mediated Rosa26:FlpoERT2 activation. FIG. 23C shows normalized ssGSEA scores on RNA-seq from LSKs purified from symptomatic Npm1^{c-Frt} Flt3^{Frt-ITD} or Dnmt3a^{Lox-R878H} Flt3^{Frt-ITD} mice; Genesets were derived from FIG. 21C. FIG. 23D shows a column-normalized heatmap of gene expression data from LSKs from Npm1^{c-Frt} Flt3^{Frt-ITD} or Dnmt3a^{Loox-R878H} Flt3^{Frt-ITD} leukemic mice (rows) with each column depicting the indicated gene. FIG. 23E shows GSEA results on Hallmark genesets juxtaposing Npm1^{c-Frt} Flt3^{Frt-ITD} vs Dnmt3a^{Loox-R878H} Flt3^{Frt-ITD} LSKs; Y-axis depicts −log 10(adjusted P value) multiplied by the sign of the normalized enrichment score (NES); Positive values indicate enrichment in Npm1^{c-Frt} Flt3^{Frt-ITD} LSKs, and negative values indicate enrichment in Dnmt3a^{Loox-R878H} Flt3^{Frt-ITD} LSKs.

FIGS. 24A-24E show immunophenotypic characterization of Dnmt3a-Flt3 and Npm1-Flt3 leukemias. FIG. 24A shows mean fluorescence intensity (MFI) of Cd34 in LSKs in bone marrow from Flt3^{Frt-ITD}, Npm1^{Frt-C} Flt3^{Frt-ITD}, and Dnmt3a^{Lox-R878H} Flt3^{Frt-ITD} mice. FIG. 24B shows a dot chart depicting % lin⁻ (left), % LSK⁺ (middle) and GMP (right) of Cd45.2 cells in bone marrow from Flt3^{Frt-ITD}, Npm1^{Frt-C} Flt3^{Frt-ITD}, and Dnmt3a^{Lox-R878H} Flt3^{Frt-ITD} mice. FIG. 24C shows UMAP depicting CyTOF-derived abundance levels of Ly6G (top left) and Cd11c (bottom left) in bone marrow from leukemic mice; Relative frequency of cells from Dnmt3a^{Lox-R878H} Flt3^{Frt-ITD} (top right) and Npm1^{Frt-C} Flt3^{Frt-ITD} (bottom right) are depicted as density contour plots. FIG. 24D shows a heatmap indicating CyTOF-derived median scaled expression value for markers (rows) in each cluster of cells (columns); Cluster abundance is depicted as a barplot on the bottom of the graph. FIG. 24E shows a stacked barplot depicting abundance of cells in clusters from FIG. 24D for Dnmt3a^{Lox-R878H} Flt3^{Frt-ITD} (left) and Npm1^{Frt-c} Flt3^{Frt-ITD} (right) leukemias.

FIGS. 25A-25C show Dre mRNA mediated activation of Flt3^{GL-ITD} with cooperating mutations. FIG. 25A shows a schema representing experimental setup where Ubc:CreERT2 Flt3^{GL-ITD} mice were treated with TAM to activate the accompanying Cre-inducible allele in Npm1^{Lox-C}, Idh2^{Lox-R140Q}, or Tet2^{Flox/Flox}; Eight weeks after TAM treatment, mice were euthanized and lin⁻ HSPCs were isolated for Dre mRNA electroporation; Cells (20,000-100,000) were then transplanted into lethally CD45.1 recipients along with 200,000 whole bone marrow from Cd45.1 mice; Disease development was monitored by complete blood count. FIG. 25B shows WBC (K/μL) from mice as described in FIG. 25A. FIG. 25C shows results following bone marrow isolation and H&E staining from mice described in FIG. 25A when symptomatic (or at 20 weeks for Flt3^{GL-ITD} only mice) (400×).

FIGS. 26A-26D show immunophenotypic characterization of sequential mutagenesis models. FIG. 26A shows flow cytometric analysis in the peripheral blood at the end of the first transplant (20 weeks post Dnmt3a^{Lox-R878H}/Cre, 16 weeks post Npm1^{Frt-C}/Flp) for total myeloid cells (Left, % Cd11b+ of total Cd45.2), monocytic cells (middle, % Gr1^{mid} of Cd45.2), and granulocytic cells (right, % Gr1^{high} of Cd45.2). FIG. 26B shows flow cytometric analysis in the peripheral blood 4 weeks after the second transplant (4 weeks after Flt3^{GL-ITD}/Dre or mock electroporation) for cKIT+ cell (Left), monocytic cells (middle, % Gr1^{mid} of Cd45.2), and granulocytic cells (right, % Gr1^{high} of Cd45.2). FIG. 26C shows a strip chart from mice depicted in FIG. 23C with y-axis indicating % GMPs within the CD45.2 compartment of cells for the indicated genotypes. FIG. 26D shows mean fluorescent intensity (MFI) of Flt3 protein within the LSK compartment for the indicated genotypes.

FIGS. 27A-27I show inducible recombinases for modeling Flt3 activation. FIG. 27A shows results from experiments in which Lin bone marrow from Rosa26:RLTG mice were infected with MSCV:DreSTAPL(ODC)-IRES-GFP virus with and without the ODC degron and treated with 10 μM asunaprevir (ASV); Barplot depicting recombination (% TdTomato+from Rosa26:RLTG). FIG. 27B shows results from experiments in which, as in FIG. 27A, cells were infected with the DreSTAPL-ODC virus and treated with a panel of NS3 inhibitors including ASV 10 μM, GZV 10 μM, glecaprevir (GLV) 10 μM, and danoprevir (DPV) 10 μM; Cells were also treated with NS3 inhibitors which were predicted to show reduced inhibitory efficacy given the mutations present in the DreSTAPL construct, including Boceprevir (BCV) 10 μM, Telaprevir (TPV) 10 μM, and Simeprevir (SMV) 10 μM. FIG. 27C shows results in which bone marrow was isolated 6 days after 5-FU treatment from Npm1^{Frt-C} Flt3^{Frt-ITD} mice and infected with MSCV:FlpoDHFR IRES GFP virus; Cells were transplanted with 200,000 Cd45.1 WT cells into lethally irradiated recipients and engraftment was monitored by flow cytometry; Mice were treated with and without the FlpoDHFR stabilizing agent trimethoprim (TMP); Representative flow plot indicating accumulation of cKIT+ cells in the peripheral blood following TMP-treatment. FIG. 27D shows a schematic representation of CAG:DreSTAPL-ODC WPRE knock-in into the H11 locus (top) or P2A-DreSTAPL-ODC knock-in into the terminal exon of the Vav1 locus (bottom) replacing the endogenous stop codon. FIG. 27E shows results from experiments in which DreSTAPL-ODC knock-in mice from FIG. 27D were crossed to the Rosa26:RLTG reporter and treated with GZV; Recombination was assessed by % TdTomato+ in the peripheral blood 2 weeks post GZV treatment. FIGS. 27F-27I show results in which mice from FIG. 27E were evaluated by complete blood count 6-8 weeks post GZV treatment for WBC (K/μL) (FIG. 27F), monocytes (K/μL) (FIG. 27G), platelets (K/μL) (FIG. 27H), and hematocrit (%) (FIG. 27I).

FIGS. 28A-28G show multi-recombinase models of leukemogenesis. FIG. 28A shows a schematic depicting experimental design for triple sequential mutagenesis model of leukemia. Dnmt3a^{Lox-R878}H is activated by Cre mRNA electroporation into lin⁻ cells at transplant. Subsequent activation of Npm1^{Frt-C} occurs through TAM-mediated FlpoERT2 nuclear localization, while Flt3^{GL-ITD} is activated through GZV-mediated Vav1:DreStaPL-ODC stabilization. FIG. 28B shows a stripchart depicting WBC (K/μL) in mice 4 weeks after treatment with TAM, GZV or the combination of TAM+GZV. FIGS. 28C-28D show stripcharts depicting total myeloid (FIG. 28C) and cKIT+ cells (FIG. 28D) in the peripheral blood of mice before and after (8 weeks) ligand treatment. FIG. 28E shows a stripchart depicting cKIT+ cells in the peripheral blood of Flt3^{GL-ITD}, Idh2^{Lox-R140Q} Flt3^{GL-ITD} or Npm1^{Lox-C} Flt3^{GL-ITD} mice 12 weeks after GZV treatment (or at symptomatic endpoint for Npm1^{Lox-C} Flt3^{GL-ITD} mice). FIG. 28F shows H&E staining in Npm1^{Lox-C} Flt3^{GL-ITD} (left) and Idh2^{Lox-R140Q} Flt3^{GL-ITD} (right) for bone marrow (top) and spleen (bottom) from symptomatic mice (400×). FIG. 28G shows results from experiments in which whole bone marrow cells (500,000) from symptomatic Idh2^{Lox-R140Q} Flt3^{GL-ITD} mice were transplanted into lethally irradiated recipients with 200,000 CD45.1 WT bone marrow; Line chart depicts WBC (K/μL; left) and hematocrit (%; right) at the indicated time point post transplantation.

FIGS. 29A-29K show immunophenotypic and molecular characteristics of Flt3-genetic ablation. FIG. 29A shows results from experiments in which cKIT+ cells from Rosa26:FlpoERT2 Npm1^{Frt-C} Flt3^{Frt-ITD} (data points at top) and Ubc:CreER Vav1:DreStaPL-ODC Npm1^{Lox-C} Flt3^{GL-ITD} (data points at bottom) were cultured in Stemspan supplemented with 20 ng SCF and treated with escalating doses of 4-OHT; Relative cellular abundance was assessed with PrestoBlue at 72 hours of culture. FIG. 29B shows results from experiments in which cells were cultured as in FIG. 29A with either 400 nM 4-OHT or vehicle control; Apoptosis was assessed by Annexin V and DAPI by flow cytometry. FIG. 29C shows a dot chart depicting % lin⁻ (left), % Cd11c⁺ (middle) and granulocytes (Cd11b⁺Gr1^{high}; right) of Cd45.2 cells in bone marrow from control and TAM treated mice for the indicated disease models. FIG. 29D shows results from experiments in which cKIT+ cells from leukemic Npm1^{Lox-C} Flt3^{GL-ITD} mice were plated over a bone marrow endothelial feeder layer and treated with 4-OHT (400 nM) or vehicle control; Total cell number (left) and % LSK of Cd45.2 (right) were assessed 6 days after plating. FIG. 29E shows a row-normalized heatmap of RNA-sequencing data in untreated or 7-day TAM-treated leukemic Npm1^{Lox-C} Flt3^{GL-ITD} mice. FIG. 29F shows GSEA depicting a decrease in genes found in the ‘KEGG: Glycine and Serine Threonine Metabolism’ signature in leukemic Npm1^{Lox-C} Flt3^{GL-ITD} mice treated with TAM. FIG. 29G shows immunohistochemical detection of c-MYC in bone marrow from control and TAM (7 day) treated Npm1^{Lox-C} Flt3^{GL-ITD} (400×). FIG. 29H shows serum cytokine levels for EPO from WT, untreated Npm1^{Lox-C} Flt3^{GL-ITD}, and TAM-treated (7d) Npm1^{Lox-C} Flt3^{GL-ITD} mice. FIG. 29I shows a dot chart depicting % lin⁻ of Cd45.2 cells in bone marrow from untreated Npm1^{Lox-C} Flt3^{GL-ITD}, TAM treated mice (7 days), and recurrent leukemias. FIG. 29J shows peripheral blood chimerism of (% Cd45.2) of Npm1^{Lox-C} Flt3^{GL-ITD} following the initiation of TAM-treatment. FIG. 29K shows PCR evaluating Dre-mediated inversion (top) and Cre-mediated deletion (bottom) of Flt3^{GL-ITD} in recurrent Npm1^{Lox-C} Flt3^{GL-ITD} leukemias following TAM treatment. PCR products were visualized by capillary gel electrophoresis (FIG. 29K).

FIGS. 30A-30D show the development of a GOLDI-Lox On/Off Dre/Cre Npm1 Mouse. FIG. 30A shows a schematic of Npm1 GOLDI-Lox allele. FIG. 30B shows expression levels of HoxA10, HoxB8, Meis1 (left panel) and WT and mutant Npm1 (right panel) in hematopoietic stem and progenitor cells from WT (first bar from left), a previously established Cre-inducible Npm1 mouse (second bar from left), and two Dre-inducible Npm1 GOLDI-Lox mice (third and fourth bars from left) 7 days after Cre or Dre administration, respectively. FIG. 30C shows measured cell number 7 days after exposing Dre-induced mutant Npm1 GOLDI-Lox hematopoietic cells to 4-hydroxytamoxifen (40HT) for Cre-recombinase induction using Cre⁻; Npm1GL cells as a negative control. FIG. 30D shows results from a mouse bone marrow transplant of a Dre-induced mutant Npm1 GOLDI-Lox expressing a co-mutant Nras^{G12D} allele immediately after Dre induction, in which mice were monitored for disease development. A subset of diseased mice were then treated with tamoxifen (TAM) to induce Cre and lead to reversion of the mutant Npm1 allele back to WT Npm1; measured levels of survival (left panel), cKIT⁺ cells in the peripheral blood (middle panel), and WBC counts (right panel) in mice that received TAM compared to vehicle treated mice are shown.

### DETAILED DESCRIPTION

Among other aspects, the present disclosure relates to inducible recombinase systems that leverage certain principles of recombinase-mediated exchange to allow for temporal and spatial control of gene expression. Accordingly, in some aspects, the disclosure provides nucleic acid constructs configured for reversible activation of a target gene through recombinase-mediated exchange reactions. In some aspects, the nucleic acid constructs provided herein incorporate multiple pairs of recombinase sites in a configuration that precludes exchange by a first recombinase until after exchange by a second recombinase. In this way, target gene expression can be controlled by the manner and timing in which the nucleic acid constructs are exposed to the first and second recombinases.

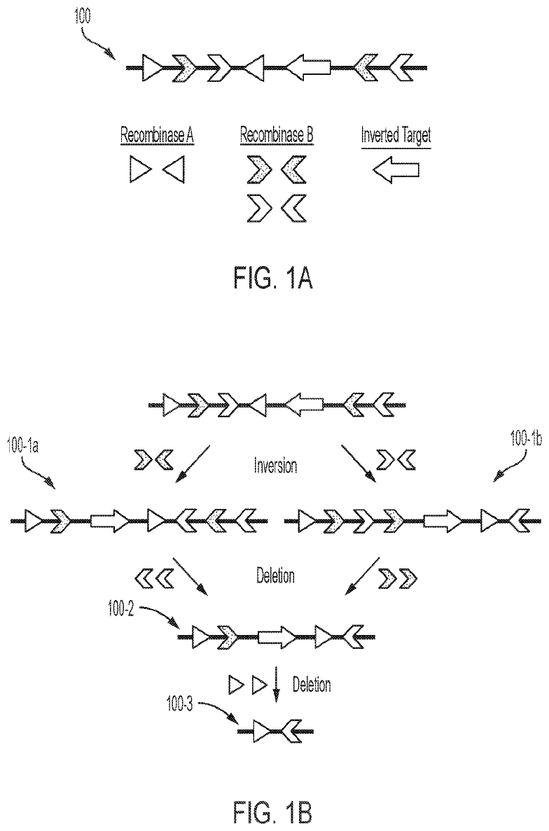

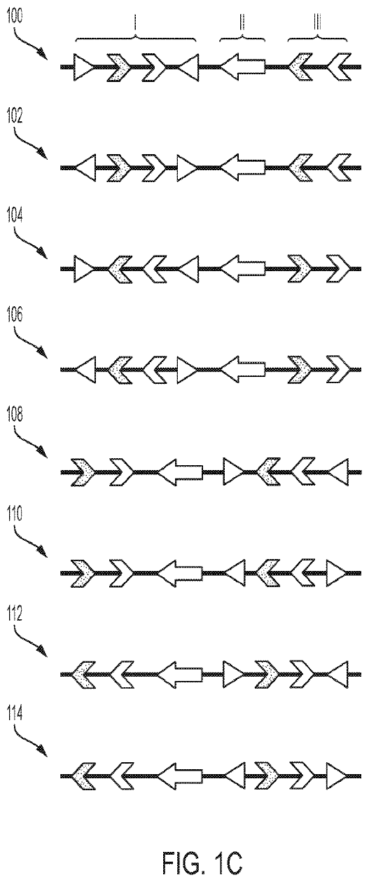

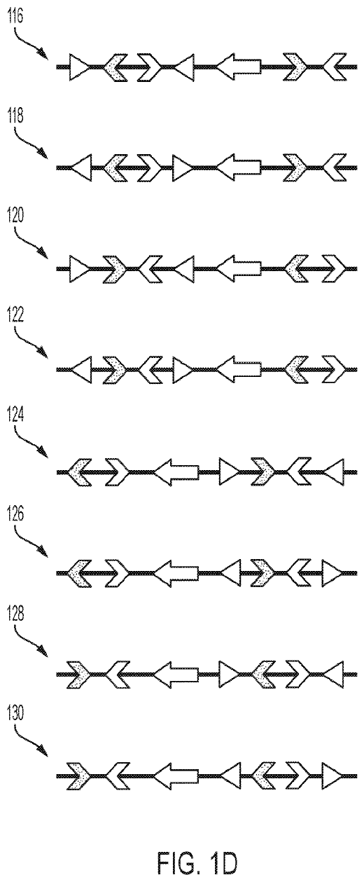

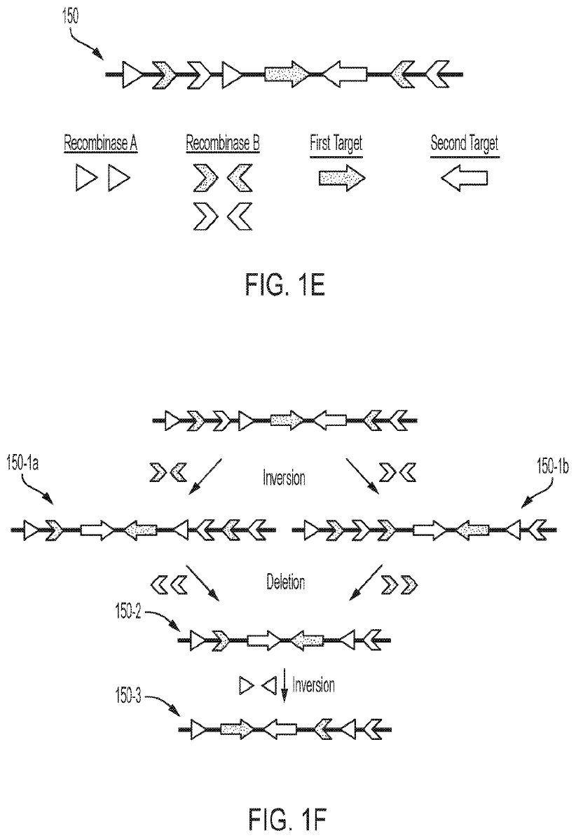

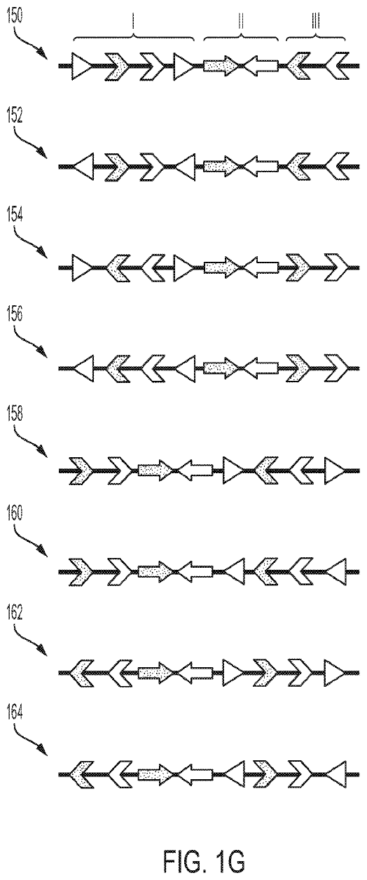

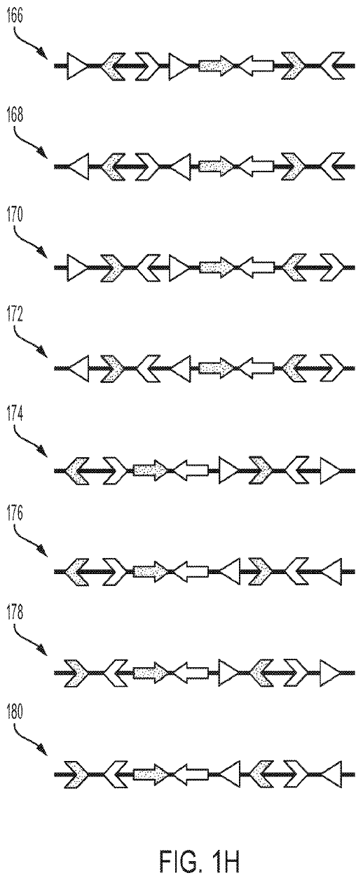

FIG. 1A shows an example configuration of a nucleic acid of the present disclosure. In this example configuration, construct 100 comprises, in 5′ to 3′ order: (a) a first member of a first pair of recombinase sites (open triangle shapes), where each member of the first pair comprises a recognition sequence for a first recombinase (“Recombinase A”); (b) a first member of a second pair of recombinase sites (stippled chevron shapes), where each member of the second pair comprises a recognition sequence for a second recombinase (“Recombinase B”); (c) a first member of a third pair of recombinase sites (open chevron shapes), where each member of the third pair comprises a recognition sequence for the second recombinase; (d) a second member of the first pair of recombinase sites; (e) an inverted expression cassette encoding a target gene (open arrow shape); (f) a second member of the second pair of recombinase sites; and (g) a second member of the third pair of recombinase sites.

In some embodiments, nucleic acids of the present disclosure are configured such that expression of a target gene can be reversibly activated and inactivated in an inducible process mediated by recombinase activity. For example, in some embodiments, a nucleic acid construct comprises an inverted expression cassette encoding a target gene such that a protein of interest is not expressed from the target gene, and through inducible recombinase-mediated activity, the nucleic acid is reconfigured to activate expression of the protein of interest (e.g., a wild-type protein or a variant of a wild-type protein). In some embodiments, through further inducible recombinase-mediated activity, the target gene is excised from the nucleic acid to inactivate expression of the protein of interest. FIG. 1B shows a process illustrating this functionality using construct 100 as an example.

In this example process, activation of target gene expression occurs through sequential inversion and deletion reactions mediated by Recombinase B. The inversion is a reversible reaction in which Recombinase B inverts the inverted expression cassette to a sense orientation through recombination at the second or third pair of recombinase sites. The recombination can occur at the second pair of recombinase sites to invert the intervening sequence between these sites to produce construct 100-1a, or the recombination can occur at the third pair of recombinase sites to invert the intervening sequence between these sites to produce construct 100-1b.

Moving forward in the process, Recombinase B can act on construct 100-1a by deletion of the intervening sequence between the third pair of recombinase sites to produce construct 100-2. Likewise, Recombinase B can act on construct 100-1b by deletion of the intervening sequence between the second pair of recombinase sites to produce construct 100-2. In some embodiments, the target gene can be expressed from construct 100-2, which comprises the expression cassette in a sense orientation. However, as each of constructs 100-1a, 100-1b, and 100-2 comprises the expression cassette in a sense orientation, it should be appreciated that any one of these constructs can express the target gene. In some embodiments, the use of two pairs of recombinase sites for Recombinase B advantageously promotes the formation of construct 100-2 by increasing the kinetic and/or thermodynamic favorability of the inversion reaction in the forward direction.

In some embodiments, construct 100 is configured such that Recombinase A cannot recombine at the first pair of recombinase sites. For example, in some embodiments, the first and second members of the first pair are separated by a distance on construct 100 that is less than a minimum distance required for recombination at these sites by Recombinase A. In some embodiments, recombination of construct 100 mediated by Recombinase B increases the separation distance between the first and second members of the first pair of recombinase sites. As such, in some embodiments, the first and second members of the first pair are separated by a distance on construct 100-2 that meets the minimum distance required for recombination at these sites by Recombinase A.

Following the recombination mediated by Recombinase B, expression of the target gene is activated. Expression of the target gene can be inactivated upon exposure of construct 100-2 to Recombinase A, which deletes the intervening sequence, including the expression cassette, between the first pair of recombinase sites to produce the inactivated construct 100-3. Accordingly, the configuration of construct 100 permits an inducible activation and inactivation of target gene expression.

FIGS. 1C-1D show additional examples of nucleic acid constructs configured for use according to the process shown in FIG. 1B. As shown in FIG. 1C for construct 100, which is as previously described, the constructs generally include three nucleic acid segments: (I) a first nucleic acid segment comprising a first pair of recombinase sites flanking a first member of each of a second and third pair of recombinase sites, where the first and second members of the first pair of recombinase sites are in an orientation opposite one another; (II) a second nucleic acid segment comprising an inverted expression cassette encoding a target gene; and (III) a third nucleic acid segment comprising a second member of each of the second and third pair of recombinase sites.

In some embodiments, the first nucleic acid segment (I) is upstream of (e.g., 5′ to) the second nucleic acid segment (II), and the third nucleic acid segment (III) is downstream of (e.g., 3′ to) the second nucleic acid segment (II), as illustrated by: constructs 100, 102, 104, and 106 (FIG. 1C); and constructs 116, 118, 120, and 122 (FIG. 1D). In some embodiments, the third nucleic acid segment (III) is upstream of (e.g., 5′ to) the second nucleic acid segment (II), and the first nucleic acid segment (I) is downstream of (e.g., 3′ to) the second nucleic acid segment (II), as illustrated by: constructs 108, 110, 112, and 114 (FIG. 1C); and constructs 124, 126, 128, and 130 (FIG. 1D). Accordingly, it should be appreciated that terms such as “first,” “second,” or “third” used with respect to a nucleic acid segment or a pair of recombinase sites do not necessarily imply a particular ordering of these elements as they occur in a 5′ to 3′ order on a nucleic acid of the disclosure.

In some embodiments, the first and second members of the first pair of recombinase sites in the first nucleic acid segment (I) are separated by a distance that prevents inversion of an intervening sequence between the first and second members of the first pair upon exposure to the first recombinase. In some embodiments, the first and second members of the first pair are separated from one another by an intervening sequence of fewer than 100 contiguous nucleobases or base pairs. In some embodiments, the first and second members of the first pair are separated from one another by an intervening sequence of fewer than 95, fewer than 90, fewer than 85, fewer than 84, fewer than 83, fewer than 82, fewer than 81, fewer than 80, fewer than 78, fewer than 76, or fewer than 74 contiguous nucleobases or base pairs. In some embodiments, the first and second members of the first pair are separated from one another by an intervening sequence of at least 30, at least 35, at least 40, at least 45, at least 50, at least 55, at least 60, at least 65, at least 70, at least 75, or at least 80 contiguous nucleobases or base pairs (e.g., about 30-90, about 30-85, about 30-82, about 40-82, about 45-82, about 50-82, about 70-82, about 72-82, about 74-82, about 75-82 contiguous nucleobases or base pairs). In some embodiments, the intervening sequence between the first and second members can be determined based on the number of contiguous nucleobases or base pairs spanning the first nucleobase or base pair adjacent to the 3′-most nucleotide of the first member and the first nucleobase or base pair adjacent to the 5′-most nucleotide of the second member.

In some embodiments, the first and second members of each pair of recombinase sites are in an orientation opposite one another. In some embodiments, the first member of each pair of recombinase sites is in a forward orientation, and the second member of each pair of recombinase sites is in a reverse orientation, as illustrated by constructs 100 and 108. In some embodiments, the first member of each pair of recombinase sites is in a reverse orientation, and the second member of each pair of recombinase sites is in a forward orientation, as illustrated by constructs 106 and 114.

In some embodiments, the first members of the second and third pairs of recombinase sites are in the same orientation as one another, and the first member of the first pair of recombinase sites is in an opposite orientation relative to the first members of each of the second and third pairs, as illustrated by constructs 102, 104, 110, and 112. In some embodiments, the first members of the second and third pairs of recombinase sites are in an orientation opposite one another, as illustrated by the constructs shown in FIG. 1D.

FIG. 1E shows an additional example configuration of a nucleic acid of the present disclosure. In this example configuration, construct 150 comprises, in 5′ to 3′ order: (a) a first member of a first pair of recombinase sites (open triangle shapes), where each member of the first pair comprises a recognition sequence for a first recombinase (“Recombinase A”); (b) a first member of a second pair of recombinase sites (stippled chevron shapes), where each member of the second pair comprises a recognition sequence for a second recombinase (“Recombinase B”); (c) a first member of a third pair of recombinase sites (open chevron shapes), where each member of the third pair comprises a recognition sequence for the second recombinase; (d) a second member of the first pair of recombinase sites; (e) a first expression cassette encoding a first target gene (stippled arrow shape); (f) a second expression cassette encoding a second target gene (open arrow shape), where the second expression cassette is an inverted expression cassette; (g) a second member of the second pair of recombinase sites; and (h) a second member of the third pair of recombinase sites.

As described above, in some embodiments, nucleic acids of the present disclosure are configured such that expression of a target gene can be reversibly activated and inactivated in an inducible process mediated by recombinase activity. For example, in some embodiments, a nucleic acid construct is configured to express a first protein (e.g., a wild-type protein), and through inducible recombinase-mediated activity, the nucleic acid is reconfigured to activate expression of a second protein (e.g., a variant of the wild-type protein). In some embodiments, through further inducible recombinase-mediated activity, the nucleic acid is further reconfigured to inactivate expression of the second protein and return to a configuration that expresses the first protein. FIG. 1F shows a process illustrating this functionality using construct 150 as an example.

For purposes of brevity, the “target gene” in this example refers to the second target gene, which can be activated and inactivated through inducible recombinase-mediated processes. However, it should be appreciated that in the context of this embodiment, when expression of the second target gene is inactive, expression of the first target gene is active. Accordingly, construct 150 is configured to express the first target gene (“First Target”), and the example process of FIG. 1F provides an illustrative example of an inducible process of switching between expression of the first target gene and the second target gene (“Second Target”).

As shown, activation of second target gene expression occurs through sequential inversion and deletion reactions mediated by Recombinase B. The inversion is a reversible reaction in which Recombinase B inverts the first expression cassette encoding the first target gene to an antisense orientation, with concomitant inversion of the second expression cassette encoding the second target gene to a sense orientation, through recombination at the second or third pair of recombinase sites. The recombination can occur at the second pair of recombinase sites to invert the intervening sequence between these sites to produce construct 150-1a, or the recombination can occur at the third pair of recombinase sites to invert the intervening sequence between these sites to produce construct 150-1b.

Moving forward in the process, Recombinase B can act on construct 150-1a by deletion of the intervening sequence between the third pair of recombinase sites to produce construct 150-2. Likewise, Recombinase B can act on construct 150-1b by deletion of the intervening sequence between the second pair of recombinase sites to produce construct 150-2. In some embodiments, the second target gene can be expressed from construct 150-2, which comprises the second expression cassette in a sense orientation. However, as each of constructs 150-1a, 150-1b, and 150-2 comprises the second expression cassette in a sense orientation, it should be appreciated that any one of these constructs can express the second target gene. In some embodiments, the use of two pairs of recombinase sites for Recombinase B advantageously promotes the formation of construct 150-2 by increasing the kinetic and/or thermodynamic favorability of the inversion reaction in the forward direction.

In some embodiments, construct 150 is configured such that Recombinase A cannot recombine at the first pair of recombinase sites. For example, in some embodiments, the first and second members of the first pair are separated by a distance on construct 150 that is less than a minimum distance required for recombination at these sites by Recombinase A. In some embodiments, recombination of construct 150 mediated by Recombinase B increases the separation distance between the first and second members of the first pair of recombinase sites. As such, in some embodiments, the first and second members of the first pair are separated by a distance on construct 150-2 that meets the minimum distance required for recombination at these sites by Recombinase A.

Following the recombination mediated by Recombinase B, expression of the second target gene is activated. Expression of the second target gene can be inactivated, and expression of the first target gene reactivated, upon exposure of construct 150-2 to Recombinase A, which inverts the intervening sequence, including the first and second expression cassettes, between the first pair of recombinase sites to produce construct 150-3. Accordingly, the configuration of construct 150 permits an inducible process of switching between expression of the first target gene and the second target gene.

FIGS. 1G-1H show additional examples of nucleic acid constructs configured for use according to the process shown in FIG. 1F. As shown in FIG. 1G for construct 150, which is as previously described, the constructs generally include three nucleic acid segments: (I) a first nucleic acid segment comprising a first pair of recombinase sites flanking a first member of each of a second and third pair of recombinase sites, where the first and second members of the first pair of recombinase sites are in the same orientation; (II) a second nucleic acid segment comprising: a first expression cassette encoding a first target gene, and a second expression cassette encoding a second target gene, where the second expression cassette is an inverted expression cassette; and (III) a third nucleic acid segment comprising a second member of each of the second and third pair of recombinase sites.

In some embodiments, the first nucleic acid segment (I) is upstream of (e.g., 5′ to) the second nucleic acid segment (II), and the third nucleic acid segment (III) is downstream of (e.g., 3′ to) the second nucleic acid segment (II), as illustrated by: constructs 150, 152, 154, and 156 (FIG. 1G); and constructs 166, 168, 170, and 172 (FIG. 1H). In some embodiments, the third nucleic acid segment (III) is upstream of (e.g., 5′ to) the second nucleic acid segment (II), and the first nucleic acid segment (I) is downstream of (e.g., 3′ to) the second nucleic acid segment (II), as illustrated by: constructs 158, 160, 162, and 164 (FIG. 1G); and constructs 174, 176, 178, and 180 (FIG. 1H). Accordingly, it should be appreciated that terms such as “first,” “second,” or “third” used with respect to a nucleic acid segment or a pair of recombinase sites do not necessarily imply a particular ordering of these elements as they occur in a 5′ to 3′ order on a nucleic acid of the disclosure.

In some embodiments, the first and second members of the first pair of recombinase sites in the first nucleic acid segment (I) are separated by a distance that prevents inversion of an intervening sequence between the first and second members of the first pair upon exposure to the first recombinase. In some embodiments, the first and second members of the first pair are separated from one another by an intervening sequence of fewer than 100 contiguous nucleobases or base pairs. In some embodiments, the first and second members of the first pair are separated from one another by an intervening sequence of fewer than 95, fewer than 90, fewer than 85, fewer than 84, fewer than 83, fewer than 82, fewer than 81, fewer than 80, fewer than 78, fewer than 76, or fewer than 74 contiguous nucleobases or base pairs. In some embodiments, the first and second members of the first pair are separated from one another by an intervening sequence of at least 30, at least 35, at least 40, at least 45, at least 50, at least 55, at least 60, at least 65, at least 70, at least 75, or at least 80 contiguous nucleobases or base pairs (e.g., about 30-90, about 30-85, about 30-82, about 40-82, about 45-82, about 50-82, about 70-82, about 72-82, about 74-82, about 75-82 contiguous nucleobases or base pairs). In some embodiments, the intervening sequence between the first and second members can be determined based on the number of contiguous nucleobases or base pairs spanning the first nucleobase or base pair adjacent to the 3′-most nucleotide of the first member and the first nucleobase or base pair adjacent to the 5′-most nucleotide of the second member.

In some embodiments, the first and second members of the second pair of recombinase sites are in an orientation opposite one another, and the first and second members of the third pair of recombinase sites are in an orientation opposite one another. In some embodiments, the first members of the second and third pairs are in a forward orientation, and the second members of the second and third pairs are in a reverse orientation, as illustrated by constructs 150, 152, 158, and 160. In some embodiments, the first members of the second and third pairs are in a reverse orientation, and the second members of the second and third pairs are in a forward orientation, as illustrated by constructs 154, 156, 162, and 164. In some embodiments, the first members of the second and third pairs of recombinase sites are in an orientation opposite one another, as illustrated by the constructs shown in FIG. 1H.

### Recombinases

In some aspects, the disclosure provides a nucleic acid comprising multiple pairs of recombinase sites. A recombinase is a genetic recombination enzyme that catalyzes directionally sensitive nucleic acid (e.g., DNA) exchange reactions between a pair of recombinase sites that are specific to a particular recombinase. Depending on the position and orientation of the pair of recombinase sites, different outcomes can result from the exchange reactions. For example, if the first and second members of a pair of recombinase sites are in the same orientation (e.g., both members in a forward orientation or both members in a reverse orientation), the exchange results in an excision of the nucleic acid segment flanked by the first and second members. If the first and second members of a pair of recombinase sites are in an orientation opposite one another (e.g., one member in a forward orientation and the other member in a reverse orientation), the exchange results in an inversion of the nucleic acid segment flanked by the first and second members.

In some embodiments, a recombinase site is a nucleotide sequence comprising a central spacer sequence, which determines the orientation (e.g., forward or reverse) of the recombinase site, flanked by two palindromic recognition sequences. Accordingly, in some embodiments, each member of a pair of recombinase sites comprises two recognition sequences, where each recognition sequence is specifically recognized and bound by a recombinase (e.g., a recombinase monomer or subunit). In a nucleic acid exchange reaction, each recognition sequence of a recombinase site is bound by a recombinase such that each member of a pair of recombinase sites is bound by two recombinase subunits, and the four recombinase subunits catalyze the exchange reaction.

A recombinase may be any recombinase known in the art. Non-limiting examples of recombinases include: Cre recombinase, Dre recombinase, Flp recombinase, Hin recombinase, Tre recombinase, Nigri recombinase, Panto recombinase, Vika recombinase, and phiC31 recombinase. A recombinase may be a wild-type recombinase or a variant recombinase. In some embodiments, a wild-type recombinase binds to a wild-type recognition sequence. In some embodiments, a recombinase is a variant recombinase. A variant recombinase comprises one or more substitutions (e.g., mutations, insertions, deletions) relative to a wild-type recombinase. In some embodiments, the one or more substitutions comprise at least one mutation, which may be a conservative mutation or a non-conservative mutation. A conservative mutation occurs when an amino acid of a wild-type protein is replaced with another amino acid having similar properties (e.g., charge, hydrophobicity, size). A non-conservative mutation occurs when an amino acid of a wild-type protein is replaced with another amino acid having different properties (e.g., charge, hydrophobicity, size). Substitutions may be made such that a variant recombinase recognizes a variant recognition sequence and/or such that the variant recombinase protein has superior protein expression and/or recombination activity in a specific cell type (e.g., mammalian cell, bacterial cell) or environment (e.g., high pH, low pH).

In some embodiments, a recombinase is a Cre recombinase. Cre recombinase is a tyrosine recombinase enzyme from the P1 bacteriophage. Cre recombinase catalyzes DNA recombination without cofactors (e.g., ATP) or accessory bacterial proteins and is thus able to conduct DNA recombination in a variety of cellular environments. A Cre recombinase may be a wild-type Cre recombinase or a variant Cre recombinase.

Cre recombinase catalyzes site-specific recombination between two recombinase sites known as locus of crossing over (x) in P1 bacteriophage (loxP) sites. These two loxP sites may be on the same or different DNA strands. LoxP sites are 34-base pair (bp) sites consisting of two 13-bp palindromic recognition sequences that flank an asymmetric 8-bp spacer sequence. One Cre protein binds to each palindromic sequence of each member of a pair of loxP sites, and four Cre proteins form a tetramer to bring two loxP sites together for recombination. Recombination occurs within the spacer sequence of the loxP sites. During recombination, the spacer region undergoes single strand breaks, followed by strand exchange, strand migration, and resolution of the Cre-loxP complex. The asymmetric spacers ensure the unidirectionality of the Cre-loxP reaction.

The orientation of two loxP sites determines the products of Cre recombination. A DNA segment between two loxP sites oriented in the same direction will be excised as a circular loop of DNA, and a DNA segment between two loxP sites oriented in opposite directions will be inverted. Recombination between two loxP sites located on different DNA molecules produces DNA strand exchange and translocation. While there is no direct contact between Cre and the loxP spacer region, the homology of the spacer is required for efficient recombination between the two loxP recognition sequences.

In some embodiments, a Cre recombinase is a wild-type Cre recombinase. In some embodiments, a wild-type Cre recombinase binds to a wild-type loxP site (Table 1). In some embodiments, a Cre recombinase is a variant Cre recombinase. A variant Cre recombinase comprises one or more substitutions (e.g., mutations, insertions, deletions) relative to a wild-type Cre recombinase. The one or more substitutions may be made such that the variant Cre recombinase recognizes a variant loxP recognition sequence and/or such that the variant Cre recombinase protein has superior protein expression and/or recombination activity in a specific cell type (e.g., mammalian cell, bacterial cell) or environment (e.g., high pH, low pH). A variant Cre recombinase may be any variant Cre recombinase known in the art. Non-limiting examples of variant Cre recombinases include: ALSHG Cre, LNSGG Cre (Baldwin et al., (2010), Chem Biol. 10(11): 1085-1094); D7, D7L^{A3}, D7R^{B2}, D7L^{K201R}, D7R^{Q311R} (Hoersten et al., (2022), Nucleic Acids Research, 50(2): 1174-1186); and Cre-A1 (wild-type+K25R, D29R, R32E, D33L, Q35R), Cre-B1 (wild-type+E69D, R72K, L76E), Cre-A2 (Cre-A1+R337E), Cre-B2 (Cre-B1+E308R), Cre-A3 (Cre-A2+E123L), Cre-B3 (Cre-B2+E123L) (Zhang, “Engineering Cre Recombinase for Genome Engineering,” Washington University Open Scholarship).

In some embodiments, a variant Cre recombinase binds to a variant loxP site. A variant loxP site comprises one or more substitutions (e.g., mutations, insertions, deletions) relative to a wild-type loxP site. In some embodiments, the one or more substitutions comprise at least one mutation, which may be a conservative or non-conservative mutation. A mutation may occur in the upstream (5′) recognition sequence, the spacer sequence, the downstream (3′) recognition sequence, or some combination thereof. A variant loxP site may be any variant loxP site known in the art. Non-limiting examples of variant loxP sites and their sequences are provided in Table 1.

In some embodiments, a recombinase is a D6 site-specific DNA recombinase (Dre recombinase). Dre recombinase is a tyrosine recombinase enzyme from the P1 bacteriophage and is closely related to Cre recombinase, but has a distinct DNA specificity for a 32-bp recombinase site. Similar to Cre recombinase, Dre catalyzes DNA recombination without accessory bacterial proteins and can conduct DNA in mammalian cells. Dre recombinase exhibits no cross-reactivity with Cre recombinase for loxP sites. A Dre recombinase may be a wild-type Dre recombinase or a variant Dre recombinase.

Dre recombinase catalyzes site-specific recombination between two recombinase sites known as region of crossover (roxP) sites. These two roxP sites may be on the same or different DNA strands. Rox sites are 32-base pair (bp) sites consisting of two 14-bp palindromic recognition sequences flanking an asymmetric 4-bp spacer sequence. One Dre protein binds to each recognition sequence of each member of a pair of roxP sites, and four Dre proteins form a tetramer to bring two roxP sites together for recombination. Recombination occurs within the spacer sequence of the roxP sites. During recombination, the spacer region undergoes single strand breaks, followed by strand exchange, strand migration, and resolution of the Dre-roxP complex. The asymmetric spacers ensure the unidirectionality of the Dre-roxP reaction.

The orientation of the two roxP sites determines the products of Dre recombination. A DNA segment between two roxP sites oriented in the same direction will be excised as a circular loop of DNA, and a DNA segment between two loxP sites oriented in opposite directions will be inverted. Recombination between two roxP sites located on different DNA molecules produces DNA strand exchange and translocation.

In some embodiments, a Dre recombinase is a wild-type Dre recombinase. In some embodiments, a wild-type Dre recombinase binds to a roxP site (Table 1). In some embodiments, a Dre recombinase is a variant Dre recombinase. A variant Dre recombinase protein comprises one or more substitutions (e.g., mutations, insertions, deletions) relative to a wild-type Dre recombinase. The one or more substitutions may be made such that the variant Dre recombinase recognizes a variant roxP recognition sequence and/or such that the variant Dre recombinase has superior protein expression and/or recombination activity in a specific cell type (e.g., mammalian cell, bacterial cell) or environment (e.g., high pH, low pH). A variant Dre recombinase may be any variant Dre recombinase known in the art. Non-limiting examples of variant Dre recombinases are described in Nguyen et al., 2015, IOVS, 56(7).

In some embodiments, a variant Dre recombinase binds to a variant roxP site. A variant roxP site comprises one or more substitutions (e.g., mutations, insertions, deletions) relative to a wild-type roxP site. In some embodiments, the one or more substitutions comprise at least one mutation, which may be a conservative or non-conservative mutation. A mutation may occur in the upstream (5′) recognition sequence, the spacer sequence, the downstream (3′) recognition sequence, or some combination thereof. A variant roxP site may be any variant roxP site known in the art. Non-limiting examples of variant roxP sites and their sequences are provided in Table 1.

In some embodiments, a recombinase is a flippase (Flp) recombinase. Flp is a tyrosine recombinase enzyme from the 2 p plasmid of Saccharomyces cerevisiae. A Flp recombinase may be a wild-type Flp recombinase or a variant Flp recombinase.

Flp recombinase catalyzes site-specific recombination between two recombinase sites known as flippase recognition target (FRT) sites. These two FRT sites may be on the same (cis) or different (trans) DNA strands. FRT sites are 34-base pair (bp) sites consisting of two 13-bp palindromic sequences that flank an 8-bp asymmetric spacer sequence. One Flp protein binds to each palindromic sequence of each member of a pair of FRT sites, and four Flp proteins form a tetramer to bring two FRT sites together for recombination. Recombination occurs within the spacer sequence of the FRT sites. During recombination, the spacer region undergoes single strand breaks, followed by strand exchange, strand migration, and resolution of the Flp-FRT complex. The asymmetric spacers ensure the unidirectionality of the Flp-FRT reaction.

The orientation of these two FRT sites determines the products of Flp recombination. A DNA segment between two FRT sites oriented in the same direction will be excised as a circular loop of DNA, and a DNA segment between two FRT sites oriented in opposite directions will be inverted. Recombination between two FRT sites located on different DNA molecules produces DNA strand exchange and translocation.

In some embodiments, a Flp recombinase is a wild-type Flp recombinase. A wild-type Flp recombinase binds to a Frt site (Table 1). In some embodiments, a Flp recombinase is a variant Flp recombinase. A variant Flp recombinase protein comprises one or more substitutions (e.g., mutations, insertions, deletions) relative to a wild-type Flp recombinase.

The one or more substitutions may be made such that the variant Flp recombinase recognizes a variant Frt recognition sequence and/or such that the variant Flp recombinase has superior protein expression and/or recombination activity in a specific cell type (e.g., mammalian cell, bacterial cell) or environment (e.g., high temperature, low temperature, high pH). A variant Flp recombinase may be any variant Flp recombinase known in the art. Non-limiting examples of variant Flp recombinases include: Flpe (Buchholz et al., 1998, Nat Biotechnol, 16(7): 657-662); Flpo (Kranz et al., 2010, Genesis, 48(8): 512-520); Flp^{H305P}, Flp^{H305L}, Flp^{R308G}, Flp^{H305E} (Parsons et al., 1988, Mol Cell Biol., 8(8): 330-3310); and FV1, FV2, FV3, FV4, FV5, FV6, FV7, FV8 (Bolusani et al., 2006, Nuc Acids Res., 34: 5259-5269).

In some embodiments, a variant Flp recombinase binds to a variant Frt site. A variant Flp site comprises one or more substitutions (e.g., mutations, insertions, deletions) relative to a wild-type Frt site. In some embodiments, the one or more substitutions comprise at least one mutation, which may be a conservative or non-conservative mutation. A mutation may occur in the upstream (5′) recognition sequence, the spacer sequence, the downstream (3′) recognition sequence, or some combination thereof. A variant Frt site may be any variant Frt site known in the art. Non-limiting examples of variant Frt sites and their sequences are provided in Table 1.

TABLE 1
Example Recombinase Sites

| | | | |
|---|---|---|---|
| | First | | Second |
| | Recognition | Spacer | Recognition |
| Name | Sequence | Sequence | Sequence |

lox sites

| | | | |
|---|---|---|---|
| Wild-type | ATAACTTCGTATA | NNNTANNN | TATACGAAGTTAT |
| loxP | (SEQ ID NO: 1) | | (SEQ ID NO: 2) |
| lox511 | ATAACTTCGTATA | ATGTATAC | TATACGAAGTTAT |
| | (SEQ ID NO: 3) | | (SEQ ID NO: 4) |
| lox5171 | ATAACTTCGTATA | ATGTGTAC | TATACGAAGTTAT |
| | (SEQ ID NO: 5) | | (SEQ ID NO: 6) |
| lox2272 | ATAACTTCGTATA | AAGTATCC | TATACGAAGTTAT |
| | (SEQ ID NO: 7) | | (SEQ ID NO: 8) |
| lox71 | TACCGTTCGTATA | NNNTANNN | TATACGAAGTTAT |
| | (SEQ ID NO: 9) | | (SEQ ID NO: 10) |
| lox72 | TACCGTTCGTATA | NNNTANNN | TATACGAACGGTA |
| | (SEQ ID NO: 11) | | (SEQ ID NO: 12) |
| lox66 | ATAACTTCGTATA | NNNTANNN | TATACGAACGGTA |
| | (SEQ ID NO: 13) | | (SEQ ID NO: 14) |
| loxPsym | ATAACTTCGTATA | ATGTACAT | TATACGAACGGTA |
| | (SEQ ID NO: 15) | | (SEQ ID NO: 16) |
| loxF8 | ATAAATCTGTGGA | AACGCTGC | CACACAATCTTAG |
| | (SEQ ID NO: 17) | | (SEQ ID NO: 18) |
| loxF8L | ATAAATCTGTGGA | AACGCTGC | TCCACAGATTTAT |
| | (SEQ ID NO: 19) | | (SEQ ID NO: 20) |
| loxF8R | CTAAGATTGTGTG | AACGCTGC | CACACAATCTTAG |
| | (SEQ ID NO: 21) | | (SEQ ID NO: 22) |
| loxM1 | ATAACTTCATATA | GCATACAT | TATATGAAGTTAT |
| | (SEQ ID NO: 23) | | (SEQ ID NO: 24) |
| loxM2 | ATAACTTCGTATA | AGAAACCA | TATACGAAGTTAT |
| | (SEQ ID NO: 25) | | (SEQ ID NO: 26) |
| loxM3 | ATAACTTCGTATA | TAATACCA | TATACGAAGTTAT |
| | (SEQ ID NO: 27) | | (SEQ ID NO: 28) |
| loxM4 | ATAACTTTGTATA | GCATACAT | TATACAAAGTTAT |
| | (SEQ ID NO: 29) | | (SEQ ID NO: 30) |
| loxM5 | ATAACTTCGTGCA | GCATACAT | TGCACGAAGTTAT |
| | (SEQ ID NO: 31) | | (SEQ ID NO: 32) |
| loxM6 | ATAACTCTGTATA | GCATACAT | TATACAGAGTTAT |
| | (SEQ ID NO: 33) | | (SEQ ID NO: 34) |
| loxM7 | ATAACTTCGTATA | AGATAGAA | TATACGAAGTTAT |
| | (SEQ ID NO: 35) | | (SEQ ID NO: 36) |
| loxM8 | ATAACTCTGTGTA | GCATACAT | TACACAGAGTTAT |
| | (SEQ ID NO: 37) | | (SEQ ID NO: 38) |
| loxM11 | TACCGTTCGTATA | CGATACCA | TATACGAAGTTAT |
| | (SEQ ID NO: 39) | | (SEQ ID NO: 40) |

rox sites

| | | | |
|---|---|---|---|
| roxP | TAACTTTAAATA | ATGCCAAT | TATTTAAAGTTA |
| | (SEQ ID NO: 41) | | (SEQ ID NO: 42) |
| rox7 | TAACTTTAAATA | AGGCCAGT | TATTTAAAGTTA |
| | (SEQ ID NO: 43) | | (SEQ ID NO: 44) |
| rox8 | TAACTTTAAATA | ACGCCTCT | TATTTAAAGTTA |
| | (SEQ ID NO: 45) | | (SEQ ID NO: 46) |
| rox12 | TAACTTTAAATA | AGGCCTGT | TATTTAAAGTTA |
| | (SEQ ID NO: 47) | | (SEQ ID NO: 48) |
| rox61 | TAACTTTAAATA | AGGCCCGT | TATTTAAAGTTA |
| | (SEQ ID NO: 49) | | (SEQ ID NO: 50) |
| rox85 | TAACTTTAAATA | AGGCCGGT | TATTTAAAGTTA |
| | (SEQ ID NO: 51) | | (SEQ ID NO: 52) |

Frt sites

| | | | |
|---|---|---|---|
| Frt | GAAGTTCCTATTC | TCTAGAAA | GTATAGGAACTTC |
| | (SEQ ID NO: 53) | | (SEQ ID NO: 91) |
| Frt1 | GAAGTTCCTATTC | TCTAGATA | GTATAGGAACTTC |
| | (SEQ ID NO: 55) | | (SEQ ID NO: 54) |
| Frt2 | GAAGTTCCTATTC | TCTACTTA | GTATAGGAACTTC |
| | (SEQ ID NO: 57) | | (SEQ ID NO: 56) |
| Frt3 | GAAGTTCCTATTC | TTCAAATA | GTATAGGAACTTC |
| | (SEQ ID NO: 59) | | (SEQ ID NO: 58) |
| Frt4 | GAAGTTCCTATTC | TCTAGAAG | GTATAGGAACTTC |
| | (SEQ ID NO: 61) | | (SEQ ID NO: 60) |
| Frt5 | GAAGTTCCTATTC | TTCAAAAG | GTATAGGAACTTC |
| | (SEQ ID NO: 63) | | (SEQ ID NO: 62) |
| Frt3 | GAAGTTCCTATTC | TCATATAA | GTATAGGAACTTC |
| | (SEQ ID NO: 65) | | (SEQ ID NO: 64) |
| Frt14 | GAAGTTCCTATTC | TATCAGAA | GTATAGGAACTTC |
| | (SEQ ID NO: 67) | | (SEQ ID NO: 66) |
| Frt545 | GAAGTTCCTATTC | TCTAAAAA | GTATAGGAACTTC |
| | (SEQ ID NO: 69) | | (SEQ ID NO: 68) |
| mFRT11 | GAAGTTCCTATAG | TTTCTAGA | CTATAGGAACTTC |
| | (SEQ ID NO: 71) | | (SEQ ID NO: 70) |
| mFRT545 | GAAGTTCCTATTC | TCTAAAAA | GTATAGGAACTTC |
| | (SEQ ID NO: 73) | | (SEQ ID NO: 72) |
| mFRT11-71 | GAAGTTTCTATAG | TTTCTAGA | CTATAGAAACTTC |
| | (SEQ ID NO: 75) | | (SEQ ID NO: 74) |
| mFRT71 | GAAGTTTCTATTC | TCTAGAAA | GTATAGAAACTTC |
| | (SEQ ID NO: 77) | | (SEQ ID NO: 76) |
| FL-IL10A | AGTGATTTGATAC | TTACATGA | GTAAAGGAATTAG |
| | (SEQ ID NO: 79) | | (SEQ ID NO: 78) |
| FRTw2 | GAAGTTCCTATAC | TATCTACA | GAATAGGAACTTC |
| | (SEQ ID NO: 81) | | (SEQ ID NO: 80) |

In some embodiments, a recombinase is an inducible recombinase. In some embodiments, an inducible recombinase refers to a recombinase that is activated in the presence of a chemical, drug, or form of energy. For example, in some embodiments, a system (e.g., a cell, an organism) does not express a recombinase in the absence of an inducer. In some embodiments, the system comprises a nucleic acid encoding the recombinase, and the inducer activates expression of the recombinase. In some embodiments, the system does not comprise a nucleic acid encoding a recombinase, and the inducer is itself a nucleic acid encoding the recombinase. The activity of an inducible recombinase protein is therefore controlled by the presence (or absence) of an inducer. This permits the timing of a particular recombination event to be controlled. One may want to restrict the timing of recombination to: the desired step in a sequence of events, when favorable environmental conditions are present, when a target gene of interest is being expressed (or shut off), when the desired stage of development or evolution is occurring, or any combination thereof.

Any chemical, drug, or form of energy used in the art as an inducer may be used to induce a recombinase. Non-limiting examples of inducers include: tamoxifen, mifepristone (RU-486), rapamycin, doxycycline, ultraviolet light illumination, tetracycline, near infrared light illumination, non-photodamaging UVA light illumination, blue light illumination, red/far-red illumination, dexamethasone, triamcinolone acetonide, RU38486, dihydrotestosterone, and OH-flutamide. In some embodiments, an inducer of a recombinase is a nucleic acid encoding the recombinase. For example, in some embodiments, an inducer is a synthetic or recombinant nucleic acid (e.g., a DNA vector, an RNA transcript) that may be exogenously introduced into a system (e.g., a cell, an organism).

### Expression Cassettes

In some aspects, the disclosure provides a nucleic acid comprising an expression cassette (e.g., an inverted expression cassette) encoding a target gene. In some embodiments, an expression cassette is a segment of a nucleic acid comprising a target gene(s) and the necessary regulatory sequences for the target gene(s) to be expressed in a cell. In some embodiments, an expression cassette contains one or more of a promoter sequence, an open reading frame comprising a target gene(s), and a 3′ untranslated region (UTR) downstream of the open reading frame.

A promoter sequence may be an endogenous promoter sequence for a target gene or an exogenous promoter sequence. An endogenous promoter sequence is a promoter sequence that drives expression of a target gene under endogenous (natural) conditions. In some embodiments, a promoter sequence is an endogenous promoter sequence for a given target gene. In some embodiments, a promoter sequence comprises one or more substitutions (e.g., mutations, insertions, deletions) relative to an endogenous promoter sequence, which can optimize the activity of the endogenous promoter in a specific organism (e.g., eukaryote, prokaryote).

An exogenous promoter sequence is a promoter sequence that does not drive expression of a target gene under endogenous circumstances. An exogenous promoter may be derived from the same organism as the target gene or derived from a different organism as the target gene. An exogenous promoter may be selected for a target gene if the exogenous promoter has favorable expression characteristics (e.g., constitutive, inducible). For example, in some embodiments, an exogenous promoter is adapted for expressing genes in a specific organism (e.g., eukaryote, prokaryote). An exogenous promoter may be any exogenous promoter known in the art. Non-limiting examples of exogenous promoters include: SV40, CAG, PGK, TRE, U6, EF1a, UAS, T7, Sp6, lac, araBad, trp, Ptac, Ubc, human beta actin, Ac5, polyhedrin, CaMKIIa, GAL1, GAL10, TEF1, GDS, ADH1, CaMV35S, Ubi, H1, T7lac, pL, and T3.

A target gene may be any gene (or portion thereof) known in the art. A nucleic acid provided herein may contain an expression cassette comprising multiple target genes. Multiple target genes may be in tandem in an expression cassette or separated by intervening sequences. In some embodiments, an expression cassette contains 1-25 target genes, 2-24 target genes, 3-23 target genes, 4-22 target genes, 5-21 target genes, 6-20 target genes, 7-19 target genes, 8-18 target genes, 9-17 target genes, 10-16 target genes, 11-15 target genes, or 12-14 target genes. In some embodiments, an expression cassette contains 1, 2, 3, 4, 5, 6, 7, 8, 9, 10, 11, 12, 13, 14, 15, 16, 17, 18, 19, 20, 21, 22, 23, 24, 25, or more target genes.

An expression cassette provided herein, in some embodiments, comprises a 3′ untranslated region (3′UTR). In some embodiments, a 3′UTR immediately follows a termination codon of a target gene (or final target gene). In some embodiments, a 3′UTR comprises one or more regulatory regions that regulate target gene expression, including polyadenylation, translation efficiency, localization, and stability of the transcribed target gene. A 3′UTR may also comprise binding sites for regulatory proteins and microRNAs (miRNAs). miRNAs decrease gene (e.g., target gene) expression of various target genes by either inhibiting their translation or directly causing degradation of a transcript of a target gene(s).

In some embodiments, a 3′UTR comprises one or more regulatory sequences, including, but not limited to, AU-rich elements (AREs), a polyadenylation sequence, a poly(A) binding protein sequence, a cytoskeleton protein binding sequence, or a combination thereof. AREs are bound by proteins that regulate the stability or decay of transcripts (e.g., transcribed from target genes) to regulate translation initiation. A polyadenylation sequence (e.g., AAUAAA) directs the addition of several hundred adenine bases to the 3′ end of a transcript (e.g., mRNA transcript of a target gene(s)) to form a poly(A) tail. A poly(A) tail contains a poly(A) binding protein (PABP) sequence. PABP binding to a poly(A) tail regulates mRNA translation, stability, and export. A cytoskeleton protein binding sequence is bound by a cytoskeleton protein (e.g., actin, myosin), which regulates transport and localization of a target gene transcript (e.g., mRNA transcript) within a cell.

### Target Genes

Provided herein, in some aspects, is a nucleic acid comprising a nucleotide sequence encoding a target gene. In some embodiments, a target gene is a gene whose expression is regulated by nucleic acids and methods provided herein. A target gene may be a complete gene, a partial gene, multiple complete genes, multiple partial genes, or some combination thereof. In some embodiments, a target gene is an exon. In some embodiments, a target gene is a nucleotide sequence encoding a protein of interest.

In some embodiments, a target gene comprises a disease-associated mutation or multiple disease-associated mutations. For example, in some embodiments, a target gene encodes a protein that comprises one or more disease-associated mutations relative to a wild-type protein. A disease-associated mutation(s) is a mutation(s) in a target gene whose expression is associated with a disease. Expressing the disease-associated mutation may cause the disease to occur in an organism (e.g., non-human mammal) with the nucleic acid containing the target gene. In some embodiments, a disease-associated mutation is necessary to cause the disease. In some embodiments, a disease-associated mutation is sufficient to cause the disease. In some embodiments, a disease-associated mutation is necessary and sufficient to cause the disease.

A disease-associated mutation may be any disease-associated mutation known in the art. Non-limiting examples of disease-associated mutations include: Jak2^{V617F} (myeloproliferative neoplasms: polycythemia vera, thrombocythemia, idiopathic myelofibrosis); Flt3^{ITD} (clonal hematopoiesis, CH); KRAS^{G12D}, KRAS^{G12A}, KRAS^{G12V} (pancreatic adenocarcinoma, colorectal adenocarcinoma, prostate adenocarcinoma, melanoma); TERT (glioblastoma multiforme, colorectal adenocarcinoma, thyroid adenocarcinoma, bladder cancer, melanoma); KRAS^{G12S}, KRAS^{G12R}, KRAS^{G12C} (colorectal adenocarcinoma, pancreatic adenocarcinoma); BRAF^{V600E} (colorectal adenocarcinoma, thyroid adenocarcinoma, melanoma); PIKCA^{E545Q}, PIKCA^{E545K} (colorectal adenocarcinoma, breast adenocarcinoma, bladder cancer, melanoma); TP53^{R175H} (colorectal adenocarcinoma, pancreatic adenocarcinoma, breast adenocarcinoma, melanoma); TP53^{R248P}, TP53^{R248Q} (glioblastoma multiforme, pancreatic adenocarcinoma, breast adenocarcinoma, bladder cancer, lymphoid malignancy); TP53^{282W} (prostate adenocarcinoma, pancreatic adenocarcinoma, breast adenocarcinoma, melanoma); TP53^{Y220C} (pancreatic adenocarcinoma, breast adenocarcinoma, bladder cancer, lymphoid malignancy); TP53^{R248G}, TP53^{R248W} (colorectal adenocarcinoma, prostate adenocarcinoma, prostate adenocarcinoma, breast adenocarcinoma, bladder cancer); IDH1^{R132H} (glioblastoma multiforme, prostate adenocarcinoma, lymphoid malignancy); VAV1^{D26G}, VAV1^{F69V}, VAV1^{E84D}. VAV1^{V102E}, VAV1^{E157K}, VAV1^{Y174C}, VAV1^{E175V}, VAV1^{L177R}, VAV1^{E234D}, VAV1^{E234Q}, VAV1^{D324Y}, VAV1^{H399Y}, VAV1^{K404R}, VAV1^{Q498K}, VAV1^{Q498R}, VAV1^{E391D}, VAV1^{R436S}, VAV1^{M501L}, VAV1^{M501R}, VAV1^{M501V}, VAV1^{E556D}, VAV1^{E556K}, VAV1^{A511D}, VAV1^{Q524E}, VAV1^{E633G}, VAV1^{G648C}, VAV1^{A673V}, VAV1^{R678Q}, VAV1^{R790C}, VAV1^{D797N}, VAV1^{R798P}, VAV1^{R798Q}, VAV1^{E850K}, VAV1^{K815E}, VAV1^{R822Q}, VAV1^{R822L}, VAV1^{Y826S}, VAV1^{W831L}, VAV1^{A798T}, VAV1^{G816R},VAV1^{G819S} (peripheral T-cell lymphoma); GFAP^{R79H}, GFAP^{R239C}, GFAP^{R239H}, GFAP^{R88C}, GFAP^{R88S}, GFAP^{L76F}, GFAP^{N77Y}, (Alexander disease); SOD1^{A4V}, SOD1^{A4T}, SOD1^{V7E}, SOD1^{V14M}, SOD1^{E21K}, SOD1^{G37R}, SOD1^{L38V}, SOD1^{G41S}, SOD1^{G41D}, SOD1^{H43R}, SOD1^{H46R}, SOD1^{H48E}, SOD1^{L84V}, SOD1^{G85R}, SOD1^{D90A}, SOD1^{G93A}, SOD1^{G93C}, SOD1^{G93R}, SOD1^{G93D}, SOD1^{E100G}, SOD1^{D101N}, SOD1^{D101G}, SOD1 SOD1^{L106V}, SOD1^{I112T}, SOD1^{I113T}, SOD1^{R115G}, SOD1^{D125H}, SOD1^{L144F}, SOD1^{V148G}, SOD1^{I149T} (amyotrophic lateral sclerosis); TBP^{(CAG)}₆₃ (ataxia); NLGN3^{R451C} (autism spectrum disorders); NLGN4^{186T} (autism spectrum disorders); NOTCH3^{W71C}, NOTCH3^{R69C}, NOTCH3^{R182C} (CADASIL); PMP22^{G150C} (Dejerine-Sottas); PSEN1^{M146L}, PSEN1^{H163R}, PSEN1^{A246E}, PSEN1^{L286V}, PSEN1^{C410Y}, PSEN1^{M139V}, PSEN1^{H163Y}, PSEN1^{E280A}, PSEN1^{E280G}, PSEN1^{P267S}, PSEN1^{E120D}, PSEN1^{A426P}, PSEN1^{M146I}, PSEN1^{L250S}, PSEN1^{R278T}, PSEN1^{C92S}, PSEN1^{G206A}, PSEN1^{G266S}, (Alzheimer disease); APP (early-onset dementia); PCDH19^{V441E}, PCDH19^{E85T}, PCDH19^{S671ter}, PCDH19^{E48ter}, PCDH19^{N557K} (focal epilepsy); CACNA1A^{R192Q}, CACNA1A^{T666M}, CACNA1A^{V714A}, CACNA1A^{I1811L}, CACNA1A^{D715E}, CACNA1A^{Y1385C}, CACNA1A^{S218L}, CACNA1A^{R583Q}, CACNA1A^{V1457L}, CACNA1A^{I710T}, CACNA1A^{R1347Q}, (hemiplegic migraine); SYNGAP1^{K138ter}, SYNGAP1^{R579ter}, SYNGAP1^{K138ter}, SYNGAP1^{W362R}, SYNGAP1^{P562L}, SYNGAP1^{W267ter}, SYNGAP1^{R143ter} (intellectual disability); NF1^{L348P}, NF1^{R365X}, NF1^{C5242T}, NF1^{C5260T}, NF1^{C5839T}, NF1^{AA5843del}, NF1^{R1947X}, (neurofibromatosis type II); NF2^{L360P}, NF2^{L535}, NF2^{E538P}, NF2^{F96del}, NF2^{E182ter}, NF2^{R262ter}, NF2^{E320ter}, NF2^{R431ter}, NF2^{E407ter}, NF2^{E463ter}, NF2^{R466ter}, NF2^{E527ter}, (neurofibromatosis type II); ATP1A3^{T613M}, ATP1A3^{1274T}, ATP1A3^{E227K}, ATP1A3^{1758S}, ATP1A3^{F780L}, ATP1A3^{D801R}, ATP1A3^{D923N} (rapid onset dystonia Parkinsonism); MECP2^{R133C}, MECP2^{F155S}, MECP2^{806del}, MECP2^{R270ter}, MECP2^{T158M}, MECP2^{R106W}, MECP2^{R294ter}, and MECP2^{R306C} (Rett syndrome).

In some embodiments, a target gene is Jak2 and encodes the protein Janus kinase 2 (JAK2). JAK2 is a non-receptor tyrosine kinase that regulates cytokine and growth factor signaling. Activated JAK2 recruits and phosphorylates signal transducer and activator of transcription (STAT) proteins, which translocate to the nucleus and regulate gene transcription. Dysregulation of JAK2 activity produces increased cellular proliferation and myeloproliferative neoplasms (MPNs). Jak2 may be any Jak2 known in the art. Non-limiting examples of Jak2 include: human (Gene ID: 3717), mouse (Gene ID: 16452), rat (Gene ID: 24514), cow (Gene ID: 525246), chicken (Gene ID: 374199), dog (Gene ID: 484185), rabbit (Gene ID: 100358134), frog (Gene ID: 100036599), rhesus monkey (Gene ID: 694184), chimpanzee (Gene ID: 464979), and pig (Gene ID: 397201).

In some embodiments, a target gene is Jak2^{V617F} or a corresponding mutation in a non-human. The Jak2^{V617F} mutation is present in myeloproliferative neoplasms (MPNs), with up to 97% of patients with polycythemia vera, up to 57% of patients with thrombocythemia, and up to 50% of patients with idiopathic myelofibrosis (see, e.g., Baxter, et al., 2005, Lancet, 365: 1054-1061). This Jak2^{V617F} mutation is predicted to dysregulate JAK2 kinase activity, resulting in increased cell proliferation.

In some embodiments, a target gene is Flt3 and encodes the protein fins related receptor tyrosine kinase 3 (FLT3). FLT3 is a class III receptor tyrosine kinase that regulates hematopoiesis. FLT3 is activated by binding to FLT3 ligand (FLT3L) and phosphorylates and activates multiple effectors in apoptosis, proliferation, and hematopoietic stem cell differentiation in bone marrow. Dysregulations due to mutations in Flt3 are associated with clonal hematopoiesis (CH) and leukemia. Flt3 may be any Flt3 known in the art. Non-limiting examples of Flt3 include: human (Gene ID: 2322), mouse (Gene ID: 14255), rat (Gene ID: 140635), cow (Gene ID: 512700), chicken (Gene ID: 100858422), dog (Gene ID: 486025), frog (Gene ID: 101733150), rhesus monkey (Gene ID: 721712), and chimpanzee (Gene ID: 452508).

In some embodiments, a target gene is Flt3^{ITD} or a corresponding mutation in a non-human. The Flt3^{ITD} mutation is an internal tandem duplication (ITD) in exons 14 and 15 and is present in leukemia (e.g., acute myeloid leukemia, AML). The Flt3^{ITD} mutation ranges in size from 3 to more than hundreds of nucleotides and are thought to disrupt an autoinhibitory conformation of the FLT3 receptor, resulting in constitutive activation of downstream signaling.

In some embodiments, a target gene is TP53 and encodes the protein tumor protein p53 (TP53). TP53 is a tumor suppressor protein containing transcriptional activation, DNA binding, and oligomerization domains. TP53 responds to diverse cellular stresses to regulate expression of target genes, thereby inducing cell cycle arrest, apoptosis, senescence, DNA repair, or changes in metabolism. Dysregulations in TP53 are associated with cancers. TP53 may be any TP53 known in the art. Non-limiting examples of TP53 include: human (Gene ID: 7157), mouse (Gene ID: 22059), rat (Gene ID: 24842), dog (Gene ID: 403869), pig (Gene ID: 397276), chicken (Gene ID: 396200), cow (Gene ID: 281542), cat (Gene ID: 493847), rabbit (Gene ID: 10009292), rhesus monkey (Gene ID: 716170), and chimpanzee (Gene ID: 455214).

In some embodiments, a target gene comprises TP53^{R175H}, TP53^{R248P}, TP53^{R248Q}, TP53^{282W}, TP53^{Y220C}, TP53^{R248G}, and/or TP53^{PR248W}.

In some embodiments, a target gene is KRAS and encodes the protein Kirsten rat sarcoma viral oncogene (KRAS). KRAS is an oncogene homolog that is a member of the small GTPase superfamily. A single amino acid mutation in KRAS may cause an activating mutation that is implicated in numerous cancers, including lung adenocarcinoma, mucinous adenocarcinoma, ductal carcinoma of the pancreas, and colorectal carcinoma. KRAS may be any KRAS known in the art. Non-limiting examples of KRAS include: human (Gene ID: 3845), mouse (Gene ID: 16653), rat (Gene ID: 24525), dog (Gene ID: 403871), cow (Gene ID: 541140), rhesus monkey (Gene ID: 707977), cat (Gene ID: 751104), frog (Gene ID: 100485959), chicken (Gene ID: 418207), chimpanzee (Gene ID: 473387), and pig (Gene ID: 100518251).

In some embodiments, a target gene comprises KRAS^{G12S}, KRAS^{G12R}, KRAS^{G12C}, KRAS^{G12D}, KRAS^{G12V}, KRAS^{G13D}, KRAS^{A59T}, KRAS^{G60R}, KRAS^{D1S3V}, KRAS^{T58I}, KRAS^{V141}, KRAS^{P34R}, KRAS^{V52G}, KRAS^{D153V}, KRAS^{G13R}, KRAS^{F156L}, KRAS^{G60S}, KRAS^{Y71S}, KRAS^{K147E}, KRAS^{G13C}, KRAS^{L19F}, KRAS^{A146T} and/or KRAS^{A146V}.

In some embodiments, a target gene is ASXL1 and encodes the protein additional sex combs-like 1 (ASXL1). ASXL1 is a protein that is a member of the Polycomb group of proteins which are necessary for the maintenance of stable repression of gene loci.

Specifically, ASXL1 is thought to disrupt chromatin in localized areas, enhancing transcription of certain genes while repressing the transcription of other genes. Dysregulations in ASXL1 are associated with myelodysplastic syndromes and chronic myelomonocytic leukemia (CML). ASXL1 may be any ASXL1 known in the art. Non-limiting examples of ASXL1 include: human (Gene ID: 171023), mouse (Gene ID: 228790), rat (Gene ID: 311553), rhesus monkey (Gene ID: 711799), cow (Gene ID: 522091), chimpanzee (Gene ID: 458168), chicken (Gene ID: 428158), dog (Gene ID: 100688017), and frog (Gene ID: 100379981).

In some embodiments, a target gene comprises Asxl1^{Q925ter}, Asxl1^{R404ter} Asxl1^{Q773ter}, and/or Asxl1^{R965ter}.

In some embodiments, a target gene is DNMT3a and encodes the protein DNA methyltransferase 3 alpha (DNMT3A). DNMT3A is a protein that functions in de novo methylation rather than maintenance methylation. DNA methylation is required for mammalian development. DNMT3A localizes to the cytoplasm and the nucleus and its expression is developmentally regulated. Mutations in DNMT3A are associated with acute myeloid leukemia (AML). DNMT3A may be any DNMT3A known in the art. Non-limiting examples of DNMT3A include: human (Gene ID: 1788), mouse (Gene ID: 13435), rat (Gene ID: 444984), chicken (Gene ID: 421991), cow (Gene ID: 359716), pig (Gene ID: 100037301), rhesus monkey (Gene ID: 694822) frog (Gene ID: 100496503), chimpanzee (Gene ID: 739139), dog (Gene ID: 482996), and cat (Gene ID: 100510798).

In some embodiments, a target gene comprises DNMT3A^{889TGGdel}, DNMT3A^{L648P}, DNMT3A^{M548K}, DNMT3A^{F902S}, DNMT3A^{R882H}, DNMT3A^{R882C}, DNMT3A^{W330R}, and/or DNMT3A^{D333N}.

In some embodiments, a target gene is RUNX1 and encodes a runt-related transcription factor 1 (RUNX1) protein. RUNX1 is a protein that forms half of the heterodimeric transcription factor core binding factor (CBF) that binds to the core element of many enhancers and promoters. RUNX1 is thought to be involved in hematopoiesis, and translocations in RUNX1 are associated with leukemias. RUNX1 may be any RUNX1 known in the art. Non-limiting examples of RUNX1 include: human (Gene ID: 861), mouse (Gene ID: 12394), rat (Gene ID: 50662), pig (Gene ID: 100512633), cow (Gene ID: 529631), frog (Gene ID: 100485449), chicken (Gene ID: 396152), rhesus monkey (Gene ID: 696749), chimpanzee (Gene ID: 101058316), and dog (Gene ID: 487746).

In some embodiments, a target gene comprises RUNX1^{IVS3As, G-T, -1}, RUNX1^{R201Q}, RUNX1^{K83E}, RUNX1^{Y260ter}, RUNX1^{A107P}, RUNX1^{H58N}, RUNX1^{A129E}, and/or RUNX1^{8-bp del, NT442}.

### Cells and Cellular Organisms

In some aspects, the disclosure provides a cell comprising a nucleic acid described herein. In some embodiments, the cell is a eukaryotic cell. For example, in some embodiments, the cell is a mammalian cell, a plant cell, a fungi cell, a reptilian cell, a protozoal cell, an excavata cell, or a diatom. In some embodiments, a cell is a prokaryotic cell. For example, in some embodiments, the cell is a bacterial cell, an archaeal cell, or a cyanobacterial cell.

A nucleic acid provided herein may be introduced into a cell (e.g., for expression, genomic integration) by any method known in the art. Non-limiting examples for methods of introducing a nucleic acid into a cell include, without limitation, transfection (e.g., DEAE-dextran, calcium phosphate, artificial lipids), electroporation, microinjection, viral infection (e.g., lentivirus, adeno-associated virus, adenovirus), dendrimer delivery, and lipid nanoparticle delivery.

A nucleic acid may be independent of or integrated into the genome of a cell. For example, in some embodiments, a nucleic acid is of a circular form that is separate and not expressed from the genome of a host cell. In some embodiments, a nucleic acid is integrated into and expressed from the genome of a host cell.

In some embodiments, a nucleic acid provided herein is integrated into the genome of a cell. Integration of a nucleic acid into a genome may be achieved by any method known in the art. Non-limiting methods of integrating a nucleic acid into the genome of a cell include, for example, utilizing targeted integrase proteins and utilizing programmable nuclease proteins. An integrase protein binds to an attachment sequence(s) in a genome and catalyzes the integration of an exogenous nucleic acid sequence into the genome at the attachment sequence(s). An integrase protein may be any integrase protein known in the art. Non-limiting examples of integrase proteins include, without limitation, Bxb1 bacteriophage, Φ31 bacteriophage, Bxb1 bacteriophage, ΦBT₁ bacteriophage, and R4 bacteriophage, HIV-1 (see, e.g., Xu et al., 2013, BMC Biotechnology, 13(87)). A programmable nuclease protein binds a nucleic acid (e.g., cell genome) at a target sequence, introduces cuts into the nucleic acid, and catalyzes the insertion (or deletion) of an exogenous nucleic acid sequence into the cut nucleic acid. A programmable nuclease protein may be any programmable nuclease protein known in the art. Non-limiting examples of programmable nuclease proteins include, without limitation, clustered regularly interspaced short palindromic repeats (CRISPR)/Cas nucleases, zinc finger nucleases (ZFNs), and transcription activator-like effector nucleases (TALENs) (see, e.g., Chandrasegaran et al., 2016, J Mol Biol., 428(5 Pt B); 963-989).

In some embodiments, a cell is a eukaryotic cell. A eukaryotic cell may be any eukaryotic cell. Non-limiting examples of eukaryotic cells include mammalian cells, plant cells, fungi cells, reptilian cells, protozoal cells, and diatoms. In some embodiments, a eukaryotic cell is a mammalian cell. Non-limiting examples of mammalian cells include human cells, rodent cells (e.g., mouse cells, rat cells, hamster cells), non-human primate cells (e.g., monkey cells, chimpanzee cells, orangutan cells, gibbon cells), domestic pet cells (e.g., dog cells, cat cells), and livestock animal cells (e.g., cow cells, pig cells, chicken cells, duck cells, goat cells, sheep cells).

In some aspects, the disclosure provides a non-human mammal comprising a cell described herein (e.g., a cell comprising a nucleic acid of the disclosure). A non-human mammal may be any non-human mammal provided in the art. Non-limiting examples of non-human mammals include rodents (e.g., mouse, rat, hamster, ferret), non-human primate (e.g., monkey, chimpanzee, orangutan, gibbon), domestic pets (e.g., dog, cat), and livestock animal cells (e.g., cow, pig, chicken, duck, goat, sheep).

In some embodiments, the disclosure provides a mouse comprising a cell described herein (e.g., a cell comprising a nucleic acid of the disclosure). In some embodiments, the disclosure provides a mouse comprising a nucleic acid described herein. A mouse may be any mouse known in the art. Non-limiting examples of mouse models include, without limitation, inbred mouse strains (e.g., BALB/c, C3H, C57BL/6, CBA. DBA/2, C57BL/10, AKR, A, 129, SJL), outbred mouse strains (e.g., Swiss Webster, CD-1, ICR), spontaneous mutant mouse strains (e.g., athymic nude, nonobese diabetic (NOD)), and immunodeficient mice (e.g., J:NU, NOD.Cg-Prkdc^{scid}-Il2rg^{tm1wjl}/SzJ (NSG), NOD.Cg-Prkdc^{scid}-Il2rg^{tm1Wjl} Tg(CMV-IL3, CSF2, KITLG)1Eav/MloSzj (NSG-SGM3); C57Bl/6-Tg(TcraTcrb)1100Mjb/J; NSG-CSF1; NSG-HLA-A2/HHD; NOD.Cg-Rag^{tm1Mom} Il2rg^{tm1Wjl}/SzJ; B6.129-Ptprc^{tm1Holm}/J; Rag1^{tm1Mom} Tg(TIE2-lacZ)182Sato/J; NSG-huIL-15; NOD.Cg-Flt3^{em2Mvw} Prkdc^{scid} Il2rgm^{tm1wjl} Tg(FLT3LG)7Sz/SzJ; NOD.Cg-Prkdc^{scid} Otc^{em18shep} Il2rg^{tm1Wjl}/J).

In some embodiments, a mouse provided herein is a transgenic mouse. A transgenic mouse is a mouse that expresses an exogenous nucleic acid sequence. An exogenous nucleic acid sequence is a nucleic acid sequence that is not derived from or found in a mouse. An exogenous nucleic acid sequence may be derived from any organism provided herein. In some embodiments, an exogenous nucleic acid sequence is derived from human.

Provided herein, in some embodiments, are methods of producing a non-human mammal. Non-limiting methods of producing a non-human mammal include, without limitation, pronucleus microinjection, embryonic stem cell recombination and transfer, Cre-lox genome recombination, viral vector infection, cytoplasmic injection, primordial germ cell recombination, nuclear transfer, and spermatogonial manipulation.

In some embodiments, a non-human mammal is produced using pronucleus microinjection. This method is well-known in the art (see, e.g., Weinstein, 2019, “Mouse Modeling, Part 2: Breeding and Crossing Mice,” Addgene.org, which is incorporated by reference herein). Briefly, pronucleus microinjection includes: injecting a pronucleus (e.g., mouse pronucleus) with a nucleic acid, integrating the nucleic acid into the pronuclear genome, implanting the pronuclear into a pseudopregnant female non-human mammal, sequencing the resulting non-human mammals for nucleic acid integration, and breeding non-human mammals containing the nucleic acid.

### Methods of Use

### Characterizing a Disease or Disorder

In some aspects, the disclosure provides methods of characterizing a disease or disorder using a nucleic acid, cell, and/or non-human mammal described herein. The nucleic acids, cells, and non-human mammals described herein are advantageous for characterizing a disease or disorder because induction of the disease or disorder may be controlled by knocking-in and knocking-out a target gene (e.g., comprising a disease-associated mutation). Non-limiting aspects of characterizing a disease or disorder may include: measuring gene expression, detecting cytokine production, measuring cell proliferation, and measuring cell survival. Measuring gene expression may be by any method known in the art including, but not limited to: RNA-sequencing (RNA-SEQ) and quantitative real-time PCR (qRT-PCR). Detecting cytokine production may be by any method known in the art including, but not limited to: immunohistochemistry and Western blotting. Measuring cell proliferation may be by any method known in the art including, but not limited to: immunostaining for cell proliferation proteins (e.g., PCNA, Ki-67) and nuclear staining. Measuring cell survival may be by any method known in the art including, but not limited to: tissue staining for apoptosis markers (e.g., caspase) and immunostaining for mitochondrial apoptosis proteins (e.g., Bax/Bak, caspase).

Any disease or disorder that may be induced by knocking-in and/or knocking-out a target gene may be characterized by methods provided herein. Non-limiting examples of diseases or disorders that may be characterized include: cancers, neurological diseases, autoimmune diseases, metabolic diseases, inflammatory diseases, cardiovascular diseases, mental disorders, gastrointestinal disorders, skeletomuscular diseases, genitourinary disorders, cell storage diseases, renal diseases, and hepatic diseases.

In some embodiments, a disease or disorder that is characterized by methods provided herein is a cancer. A cancer may be a primary cancer or a recurrent cancer. Non-limiting examples of cancers that may be characterized by methods provided herein include: adenoid cystic carcinoma, adrenal gland tumor, amyloidosis, anal cancer, appendix cancer, astrocytoma, ataxia-telegiectasia, Beckwith-Wiedeman syndrome, bile duct cancer, Birt-Hogg-Dube syndrome, bladder cancer, bone cancer, brain stem glioma, brain cancer, breast cancer, Carney complex, cervical cancer, colorectal cancer, Cowden Syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor (GIST), germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS-related cancer, juvenile polyposis syndrome, kidney cancer, lacrimal gland tumor, laryngeal and hypopharyngeal cancer, leukemia, Li-Fraumeni syndrome, liver cancer, lung cancer, lymphoma, Lynch syndrome, mastocytosis, medullablastoma, melanoma, meningioma, mesothelioma, multiple endocrine neoplasia type 1, multiple endocrine neoplasia type 2, multiple myeloma, MUTYH-associated polyposis, myelodysplastic syndrome, nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers syndrome, pheochromocytoma and paraganglioma, pituitary gland tumor, pleuropulmonary blastoma, prostate cancer, retinoblastoma, rhabdomyosarcoma, salivary gland cancer, sarcoma, non-melanoma skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom Macroglobulinemia, Werner syndrome, Wilms tumor, and xeroderma pigmentosum.

### Identifying Inhibitors and Treatments

In some aspects, methods provided herein comprise identifying inhibitors or treatments for a disease or disorder. A disease or disorder may be any disease or disorder provided herein. In some embodiments, an inhibitor is a compound that halts the progression or prevents the development of a disease or disorder. Non-limiting examples of types of inhibitors include: small molecule compounds, antibodies, peptides, nucleic acids, and proteins. Methods of identifying an inhibitor may depend on the type of disease or disorder, and multiple inhibitors may be utilized simultaneously. In some embodiments, a method for identifying an inhibitor of a disease or disorder comprises: (i) inverting or knocking-in a target gene comprising a disease-associated mutation in a cell or a non-human mammal comprising the cell; (ii) detecting onset of the disease or disorder; (iii) contacting the cell or non-human mammal with a putative inhibitor; and (iv) measuring proliferation or survival of the disease or disorder in the contacted cell or non-human mammal compared to a control. In some embodiments, the control is a cell or a non-human mammal in which the disease-associated mutation has been knocked-out or is otherwise absent. In some embodiments, the control is a cell or non-human mammal that has not been contacted with the putative inhibitor. If the putative inhibitor prevents or halts the progression or development of the disease or disorder, the putative inhibitor may be identified as an inhibitor of the disease or disorder.

In some embodiments, methods provided herein comprise identifying a treatment for a disease or disorder in a non-human mammal. A treatment is a compound that alleviates one or more symptoms of a disease or disorder provided herein. Non-limiting examples of symptoms that may be alleviated include: increased cell proliferation, increased cell survival, altered microenvironment (e.g., hypoxia, decreased extracellular glucose concentration, decreased extracellular amino acid concentration), decreased body weight, decreased energy, and decreased survival, all of which are relative to a non-human mammal that does not have the disease or disorder. Non-limiting examples of types of treatments may include: small molecule compounds, antibodies, peptides, nucleic acids, and proteins. Methods of identifying a treatment may depend on the type of disease or disorder, and multiple treatments may be utilized simultaneously. In some embodiments, a method for identifying a treatment for a disease or disorder comprises: (i) inverting or knocking-in a target gene with a disease-associated mutation in a non-human mammal; (ii) detecting onset of the disease or disorder; (iii) contacting the non-human mammal with a putative treatment; and (iv) measuring proliferation or survival of the disease or disorder in the contacted non-human mammal compared to a control non-human mammal in which the disease-associated mutation has been knocked-out and the non-human mammal has not been contacted with the putative treatment. If the putative treatment alleviates at least one symptom of the disease or disorder, the putative treatment may be considered a treatment of the disease or disorder.

### EXAMPLES

### Example 1: a Conditional Knock-In, Knock-Out Model of Jak2^{V617F} Myeloproliferative Neoplasm (MPN)

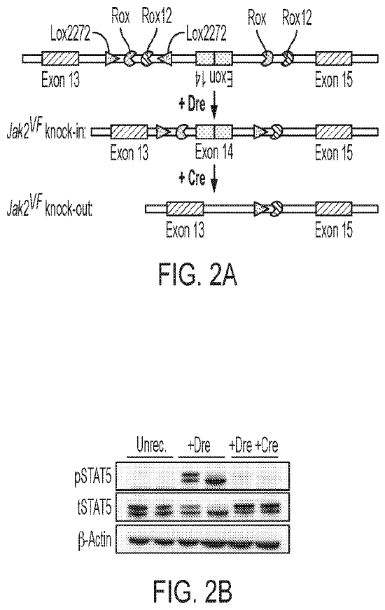

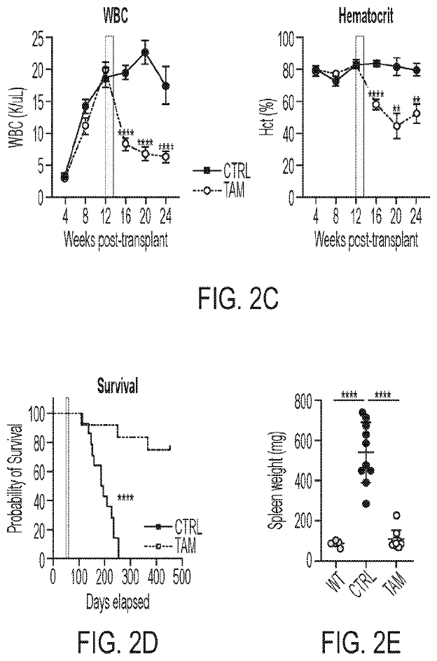

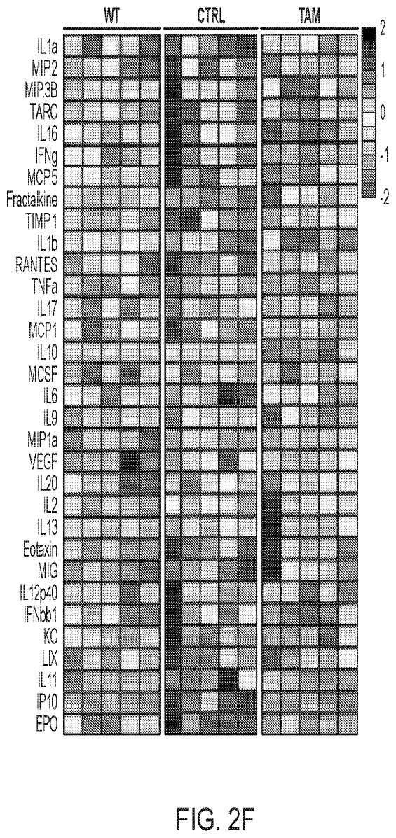

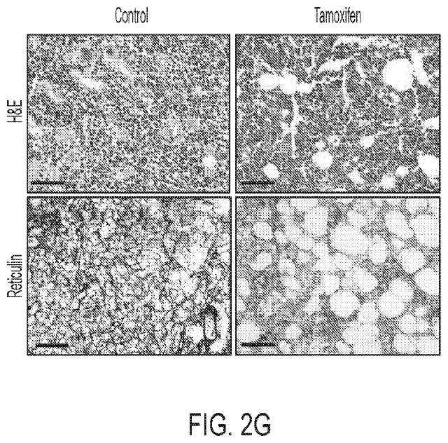

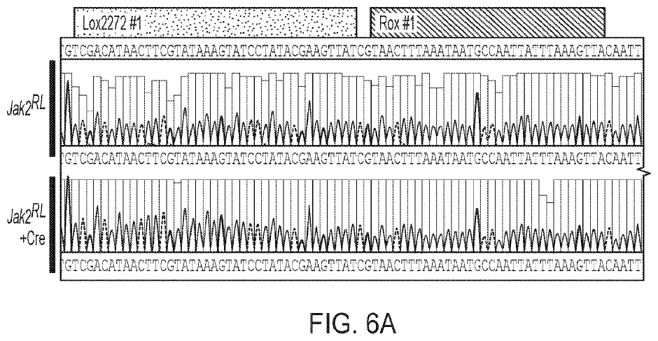

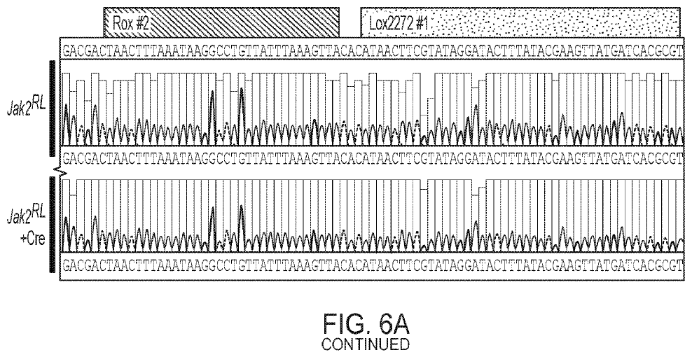

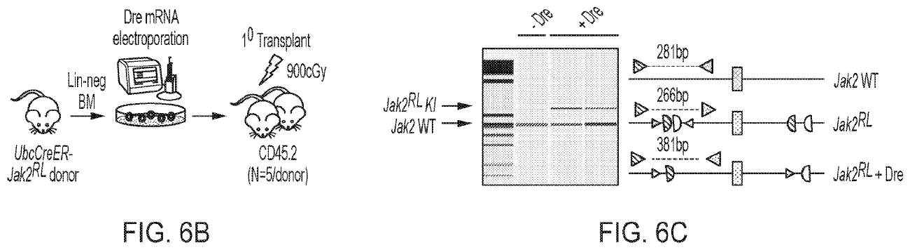

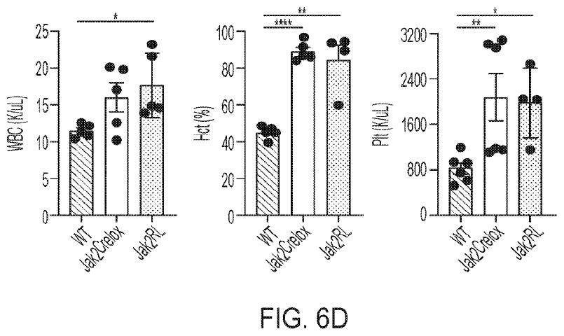

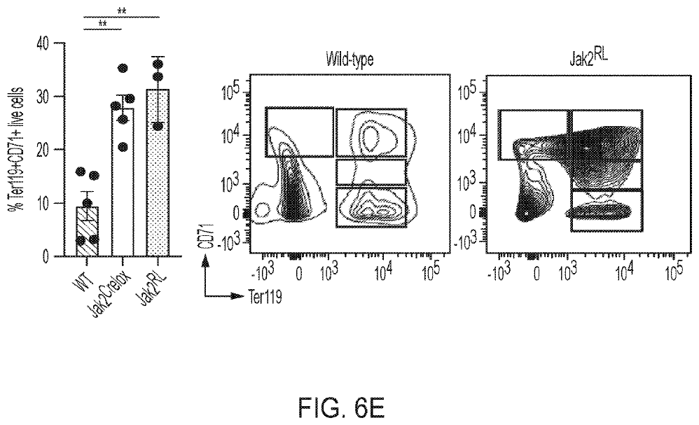

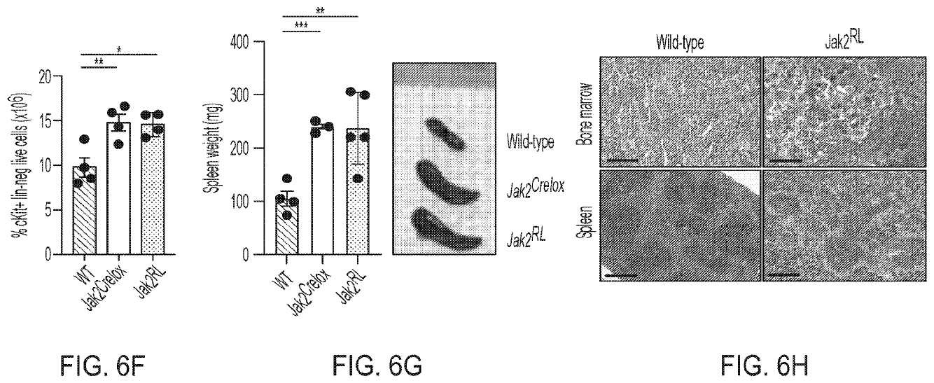

To assess dependency of MPN maintenance on JAK^{V617F} oncogenic signaling, a Dre-rox, Cre-lox dual recombinase Jak2^{V617F} knock-in/knock-out mouse model (Jak2^{Rox/Lox}/Jak2^{RL}) was generated by gene targeting in mouse embryonic stem cells (FIG. 2A). The close proximity of the lox sites (82 base pairs) prevents Cre-mediated deletion prior to Dre-mediated recombination and Jak2^{V617F} induction. Once the mutant allele is activated, the lox sites separate allowing for subsequent Cre-mediated deletion of Jak2^{V617F}, including in models where cooperating alleles are induced by antecedent Cre-mediated activation/deletion. A similar strategy, which has been termed GOLDI-Lox for governing oncogenic loci by dre inversion and lox deletion (GOLDI-Lox), was used to target FLT3^{ITD}. Sequencing of the Jak2^{RL} locus on sorted Cre reporter cells after Cre recombinase exposure confirmed retainment of the non-recombined Jak2^{RL} locus (FIG. 6A). mRNA electroporation of Dre mRNA ex vivo was used to transiently express the Dre recombinase in primary hematopoietic stem/progenitor cells (HSPCs), which efficiently induces Jak2^{V617F} activation and separation of lox sites by inversion (FIGS. 6B-6C). By three weeks post-transplant, lethally irradiated mice transplanted with Dre-electroporated Jak2^{RL} knock-in cells developed a highly penetrant and fully transplantable MPN characterized by leukocytosis with myeloid preponderance, elevated hematocrit with erythroid progenitor expansion in bone marrow, hepatosplenomegaly, and megakaryocytic hyperplasia (FIGS. 6D-6H). Progressive bone marrow fibrosis was also observed at 24 weeks in 9/15 mice consistent with prior transplant models of MPN.

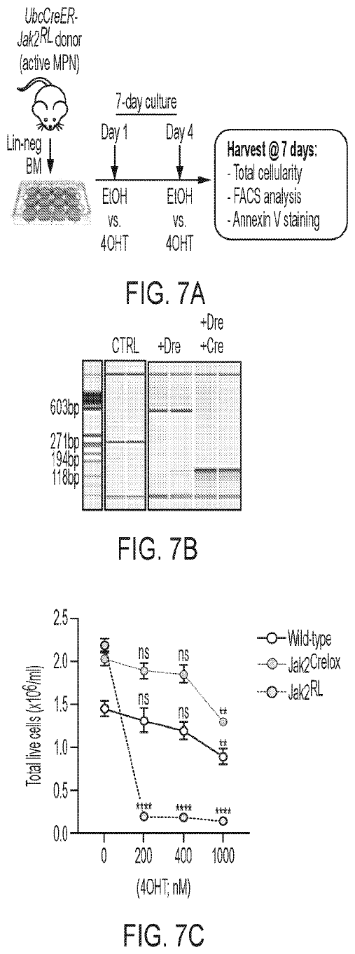

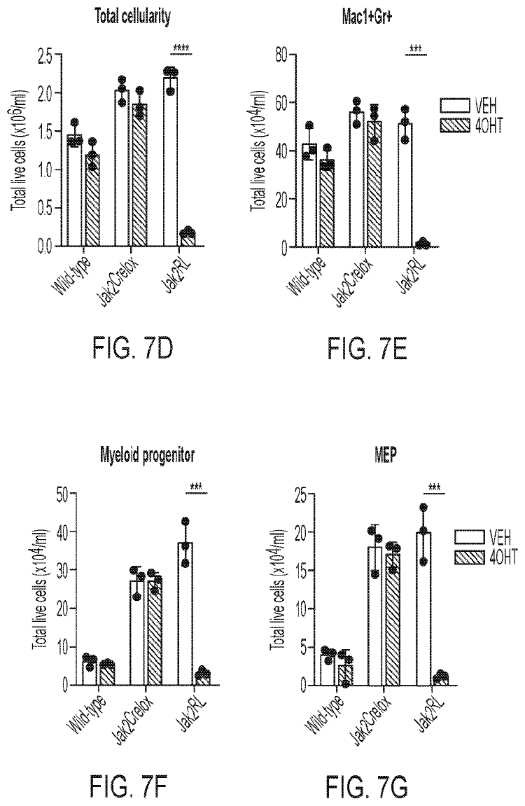

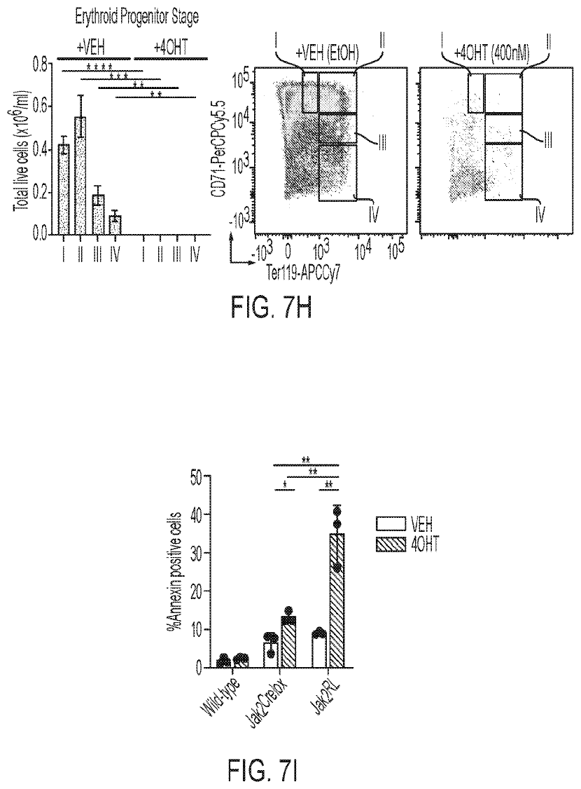

To assess the reversibility of the Jak2^{RL} construct, Dre-electroporated, lineage-negative, tamoxifen-inducible Ubc:CreER-Jak2^{RL} cells isolated from donor mice were cultured with active MPN ex vivo with increasing doses of 4-hydroxy-tamoxifen (40HT) cultured over bone marrow endothelial cells (BMECs) (FIG. 7A). Treatment with 40HT resulted in deletion of the Jak2^{V617F} allele, which was confirmed by excision polymerase chain reaction (PCR) (FIG. 7B). Loss of Jak2^{V617F} significantly reduced cell numbers ex vivo (0.18×10⁶/mL vs. 2.19×10⁶/mL, p<0.0001), including within immunophenotypically-defined HSPC compartments, a phenotypic change not observed with vehicle-treated Jak2^{RL}, Cre-inducible Jak2^{V617F} (p=0.228),⁸ or Cre-inducible wild-type (WT) cells (p=0.114) (FIGS. 7C-7G). Loss of Jak2^{V617F} also abrogated erythropoietin-independent erythroid differentiation in vitro (p<0.0001) (FIG. 7H). The cell loss observed was associated with enhanced apoptosis, which was most apparent in Mac1+ mature myeloid cells (35% vs. 9.3%, p<0.005) (FIG. 7I).

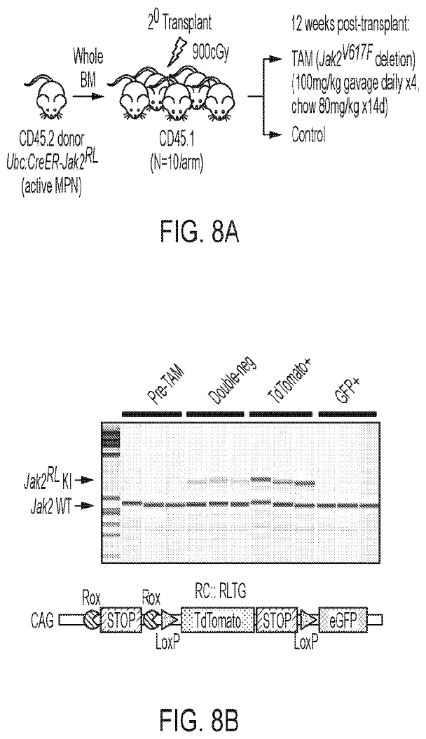

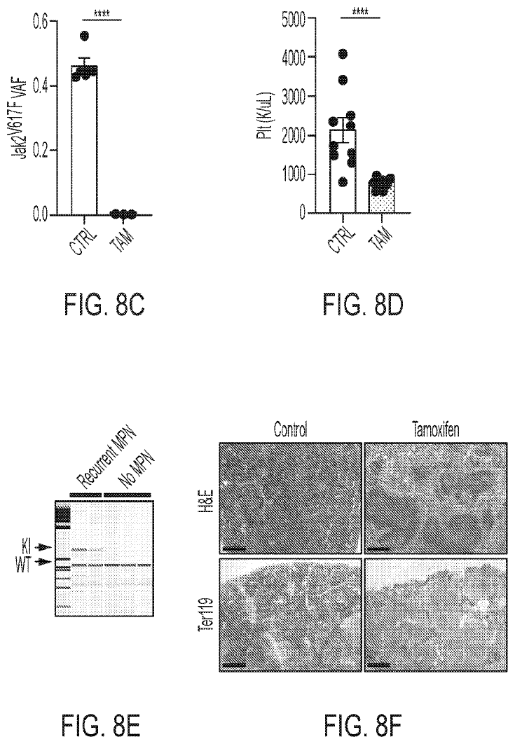

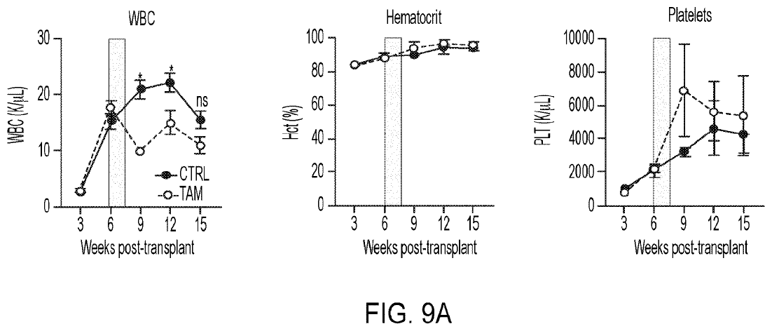

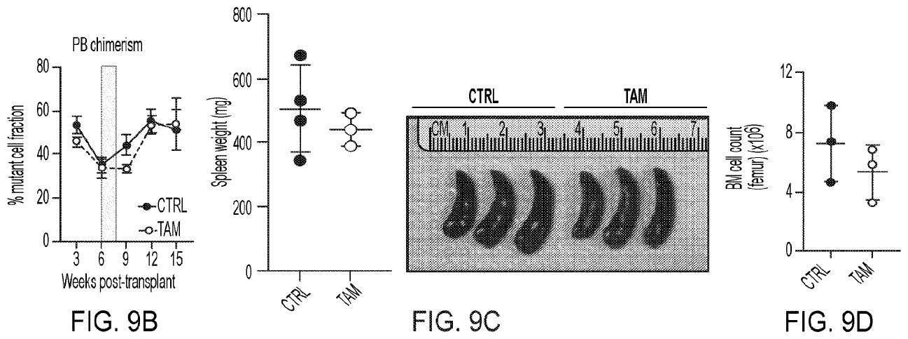

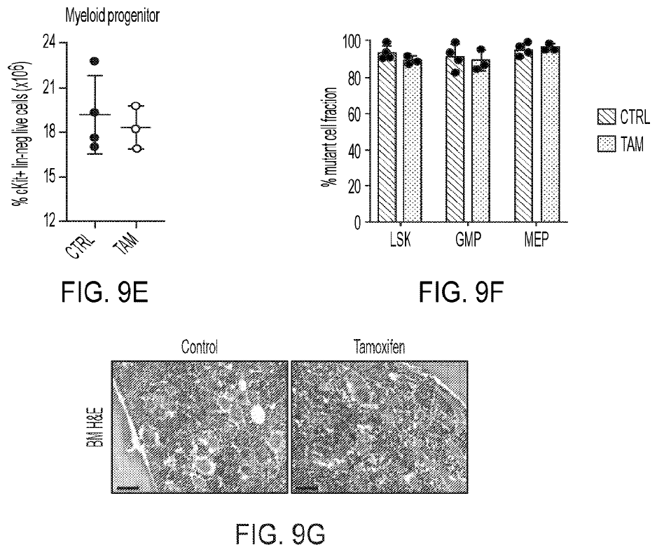

The phenotypic impact of reversible Jak2^{V617F} expression in vivo was evaluated. Three or 12 weeks post-transplant, secondary recipient mice transplanted with Dre-electroporated Ubc:CreER-Jak2^{RL} whole bone marrow in competition with CD45.1 marrow and exhibiting MPN were administered tamoxifen to delete Jak2^{V617F} (FIG. 8A). A sequential dual recombinase reporter system was used to validate Jak2^{V617F} deletion within CD45.2 reporter-positive cell populations (FIG. 8B). Deletion of Jak2^{V617F} was also validated in vivo at the transcriptional level (p<0.0001) (FIG. 8C) and was associated with loss of constitutive JAK/STAT signaling (FIG. 2B). Consistent with the in vitro data, normalization of WBC (mean 7.59 K/uL vs. 18.1 K/uL, p<0.0001), hematocrit (mean 41.6% vs. 73.5%, p<0.0001), and platelet (mean 749 K/uL vs. 1632 K/uL, p=0.0018) parameters was observed within 3-6 weeks following tamoxifen treatment that persisted until timed sacrifice at 24 weeks (FIG. 2C, FIGS. 8D-8E). Genetic reversal of Jak2^{V617F} significantly prolonged overall survival (median not defined vs. 187d, p=0.0012) and led to loss of disease penetrance in 10/15 mice (FIG. 2D). A minority of mice in the timed sacrifice cohort (3/14) demonstrated reemergence of the MPN phenotype, all of which showed incomplete excision of the Jak2^{RL} allele highlighting the necessity of Jak2^{V617F} in disease maintenance (FIG. 8E). Spleen weights (mean 108.7 mg vs. 534.7 mg, p<0.0001), bone marrow myeloid progenitor fractions, and inflammatory cytokine levels were reduced to normal levels in mice with Jak2^{V617F} reversal (FIGS. 2E-2F), and histopathologic analysis revealed reductions in megakaryocytic hyperplasia, splenic infiltration, and absence of bone marrow and spleen fibrosis in 12/14 mice consistent with near-complete pathologic regression that persisted until timed sacrifice at 24 weeks (FIG. 2G, FIG. 8F). The phenotypes observed with Ubc:CreER-Jak2^{V617F} deletion in vivo were not observed with tamoxifen administration in the absence of Jak2^{V617F} reversal (FIGS. 9A-9G). The data and observations support a conclusion that reliance of MPN phenotype requires oncogenic signaling through Jak2^{V617F}.

### Example 2: Jak2^{V617F} Reversal Impairs the Fitness of MPN Cells, Including MPN Stem Cells

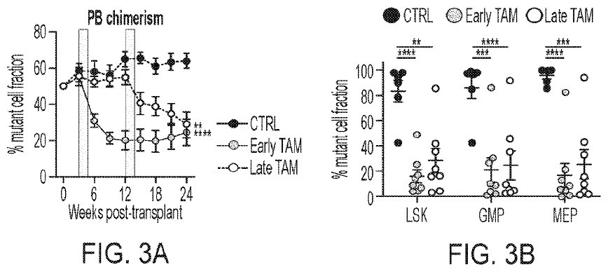

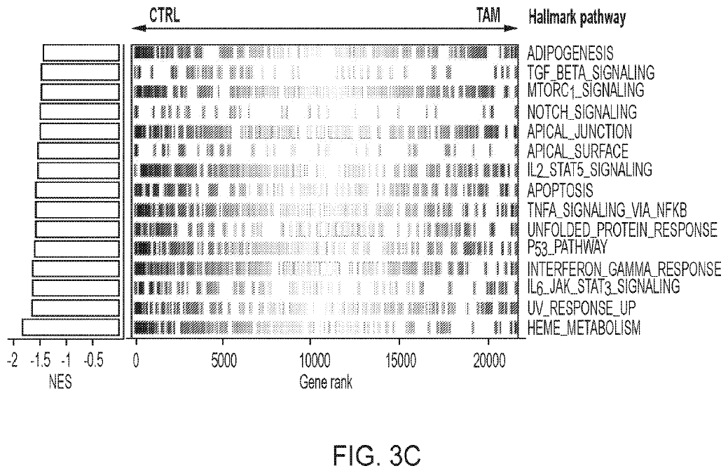

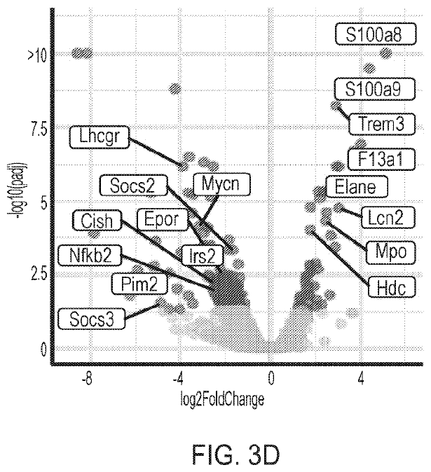

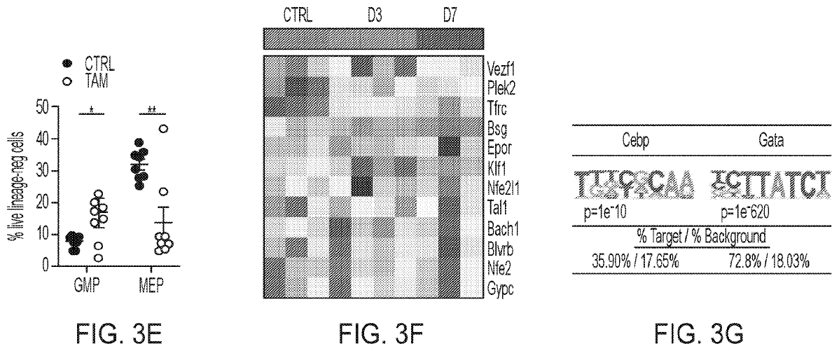

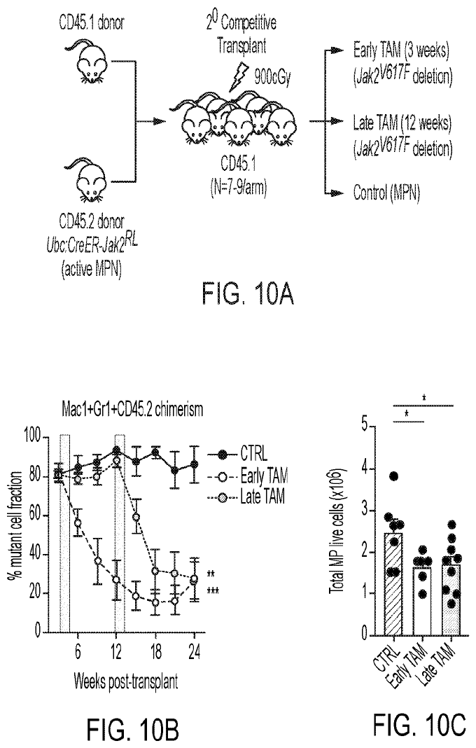

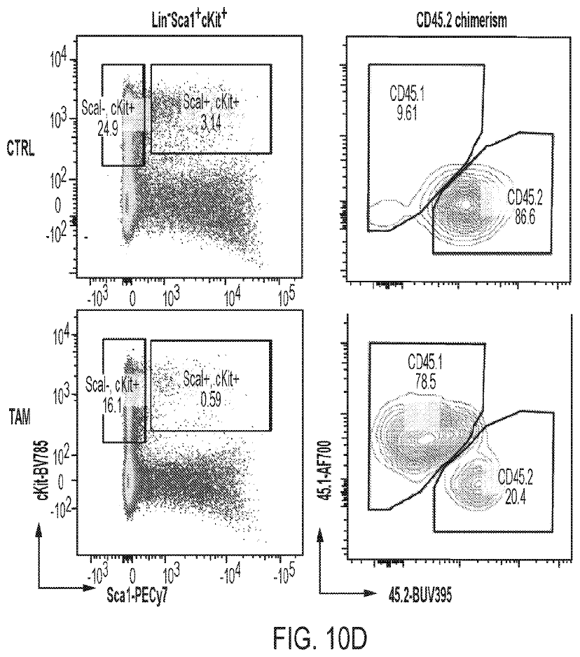

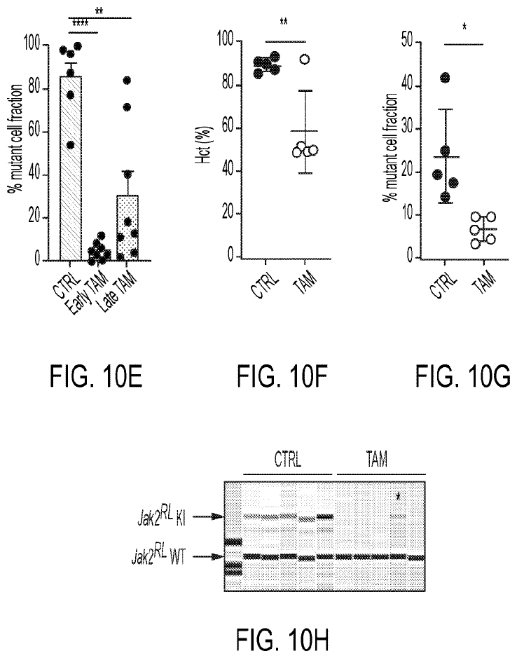

The effects of Jak2^{V617F} deletion on peripheral blood and bone marrow mutant cell fitness were evaluated in the chimeric transplant system (FIG. 10A). Abrupt, durable reductions in CD45.2 mutant cell fraction were observed in the peripheral blood (mean 24.5% vs. 63.9%, p<0.001) in response to treatment that persisted until time of sacrifice (FIG. 3A). Consistent with the in vitro data, this effect was most pronounced in Mac1+ myeloid cell fractions (p<0.0001) (FIG. 10B). In bone marrow, the reductions in mutant cell fraction in different HSPC compartments were more pronounced, including within megakaryocytic-erythroid progenitor (MEP; Lineage⁻cKit⁺Sca1⁻Cd34⁻Fcg⁻; p<0.0001) and granulocytic-monocytic progenitor (GMP; Lineage⁻cKit⁺Sca1⁻Cd34⁺Fcg⁺; p<0.0001) populations and down to the level of the LSK (Lineage⁻Sca1⁺cKit⁺; p=0.0096) stem cell compartment, including long-term hematopoietic stem cells (Lineage Sca1⁺cKit⁺CD150⁺CD48⁻) (FIG. 3B, FIGS. 10C-10D). Transplant of unfractionated Jak2^{RL} deleted bone marrow failed to form phenotypic disease in ⅘ secondary transplant recipient mice consistent with depletion of disease-propagating MPN stem cells (FIGS. 10F-10H).

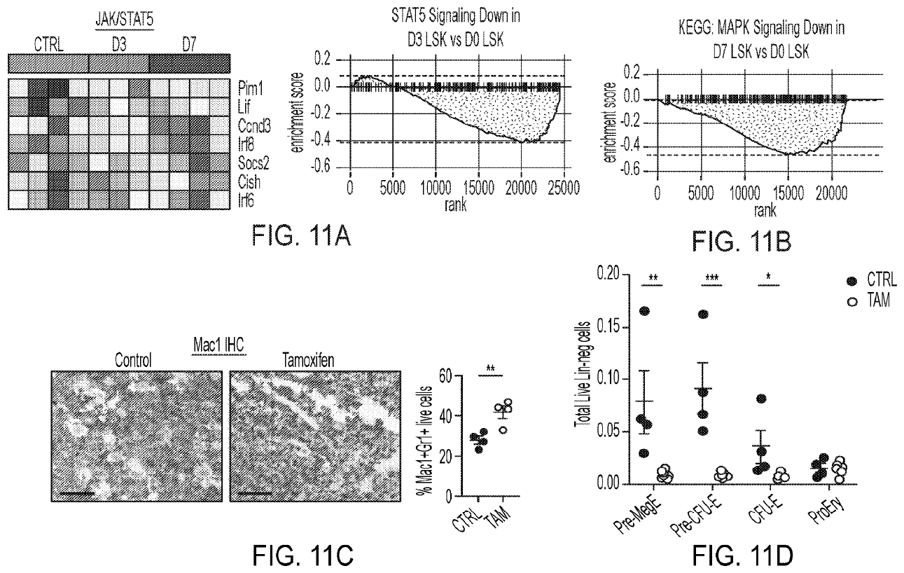

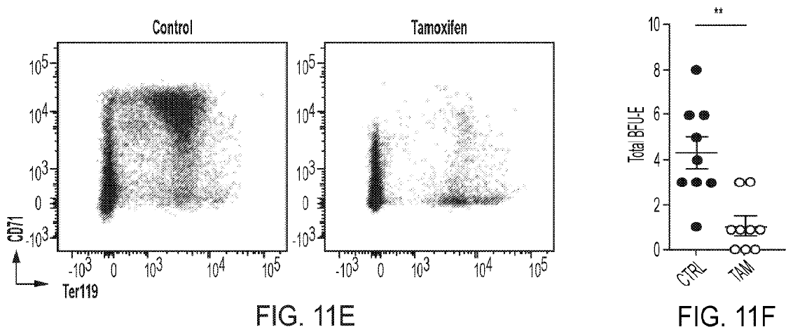

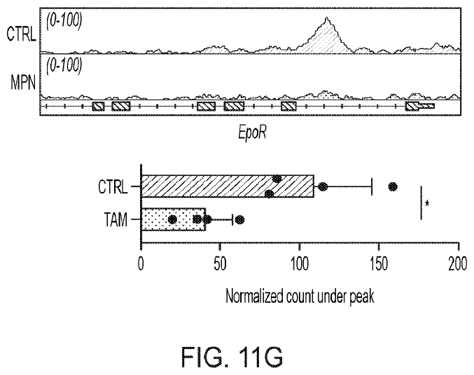

The transcriptional changes seen when Jak2^{V617F} is acutely reversed were characterized. RNA sequencing (RNA-Seq) analysis of purified HSPCs 3 and 7 days following Jak2^{V617F} deletion (n=3-4) compared to controls (n=3-4) was performed. Transcriptional analysis of sorted, Jak2^{V617F}-deleted LSK and MEP populations revealed near-complete loss of expression of STAT5 target genes as early as 3 days post-deletion (LSK: NES −1.77, padj=0.002; MEP: NES −1.53, padj=0.0065) indicating immediate disengagement from disease-defining pathway signaling (FIG. 11A). By 7 days, significant negative enrichment in interferon-gamma (NES −1.61, padj=0.0005), TGFb (NES −1.45. padj=0.071), and TNFa via NFkB (NES −1.54, padj=0.0017) Hallmark pro-inflammatory response pathways was observed, as well as down-regulation of MAPK (NES −1.52, padj=0.0052) and MTORC1 (NES −1.46, padj 0.0071) targets suggesting abrupt reduction in pro-inflammatory and proliferative signaling in the setting of Jak2^{V617F} deletion (FIG. 3C, FIG. 11B). A flux towards increased expression of myeloid genes sets compared to erythroid gene sets was also observed characterized by increased S100a8, S100a9, Mpo, and Hdc expression in LSKs, increases in GMP (14.5% vs. 7.8%, p=0.018) vs. MEP (13.8% vs. 32%, p=0.0025) frequencies within the HSPC compartment, and enrichment in Mac1+ myeloid cells (41.7% vs. 27.8%, p=0.0084) (FIGS. 3D-3E, FIG. 11C). In line with reduced erythroid output, a marked decrease in heme metabolism in MEPs with associated reductions in critical erythroid/megakaryocytic transcription factors and signaling mediators was observed, including Nfe3, Plek2, and EpoR which coincided with concomitant reductions in total erythroid progenitor cell numbers (p<0.021) and significantly reduced burst forming unit-erythroid (BFU-E) colony output of Jak2^{V617F} deleted cells (p=0.001) (FIG. 3F, FIGS. 11D-11F). Assay for Transposase Accessible Chromatin with high-throughput sequencing (ATAC-seq) on Jak2^{V617F}-deleted cKit+ cells demonstrated an increase in open chromatin with CEBP motifs (p<1×10⁻¹⁰) and reduced accessibility at Gata motifs (p<1×10⁻⁶²⁰), including at critical erythroid loci (e.g. EpoR; log 2foldchange 1.49, padj=0.00135) further consistent with an erythroid to myeloid lineage switch (FIG. 3G, FIG. 11G). Reduced accessibility at putative Gata target sites was observed; however, differential expression of either Gata1 (p=1.0) or Gata2 (p=0.82) was not observed in Jak2^{V617F}-deleted LSKs or MEPs. These data suggest the transcriptional networks regulating the MPN phenotype are not obligately achieved through transcription factor expression dysregulation but through differential transcription factor-mediated output.

### Example 3: Differential Efficacy of Jak2^{V617F} Deletion Compared to JAK Inhibitor Therapy

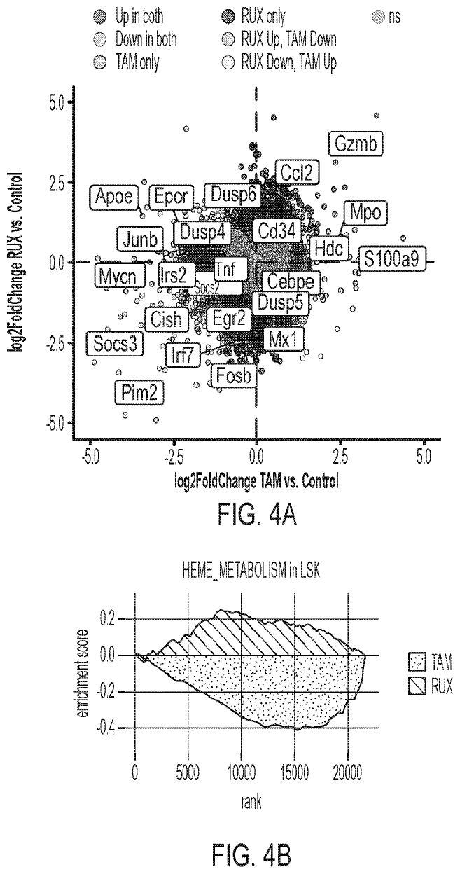

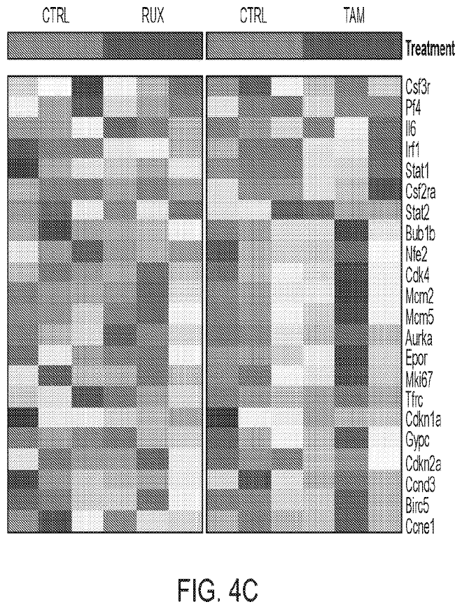

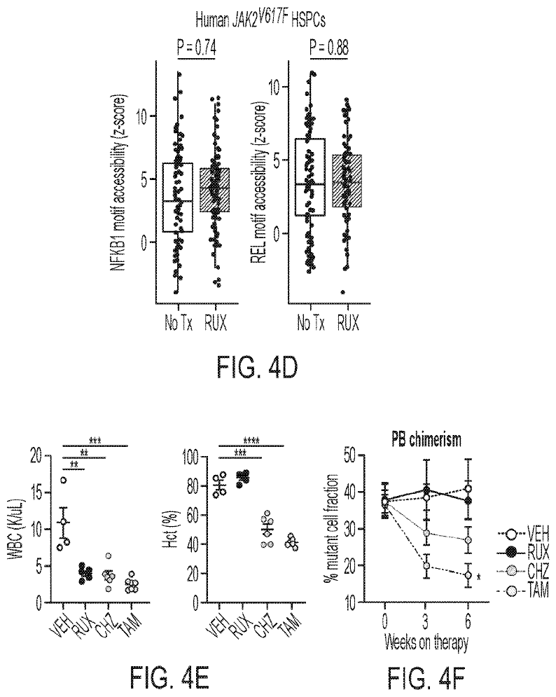

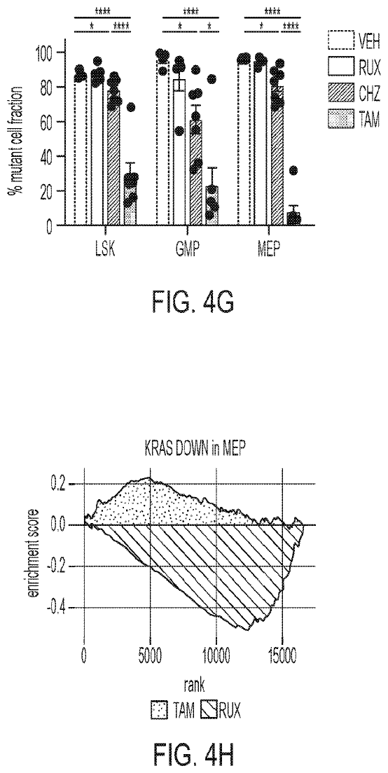

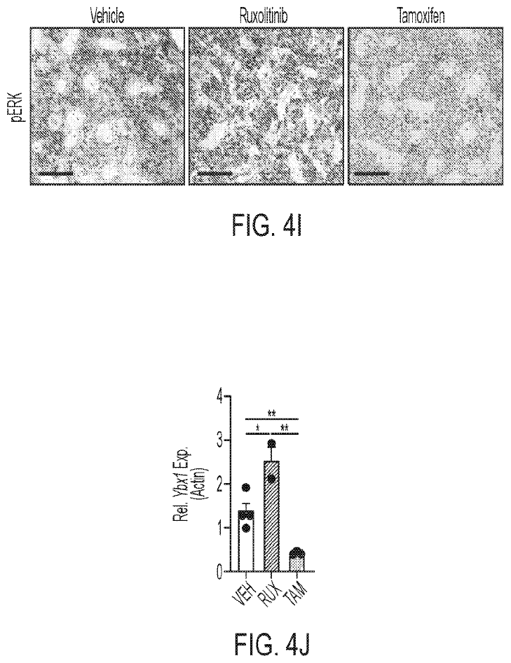

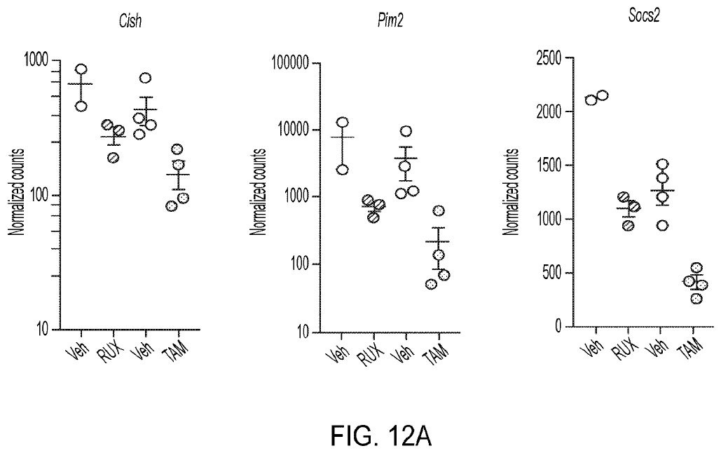

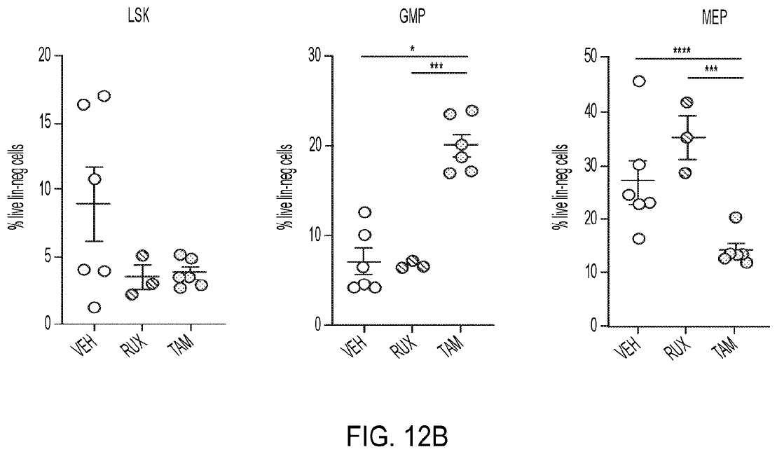

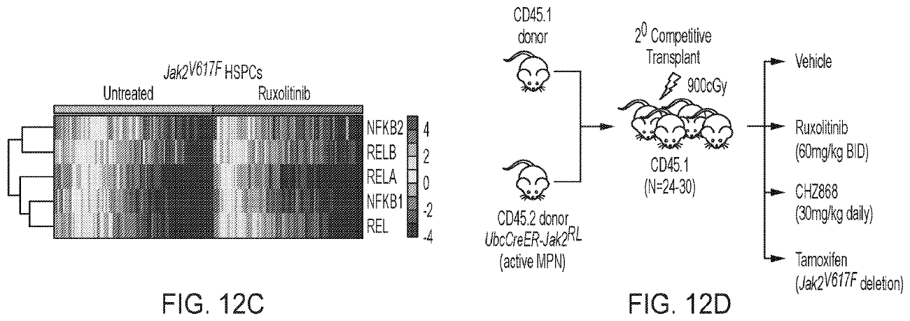

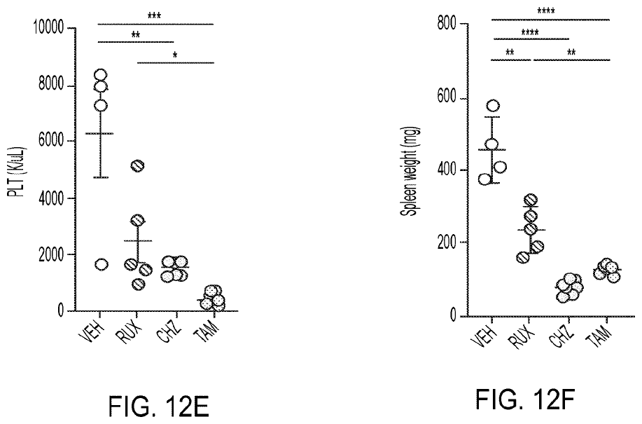

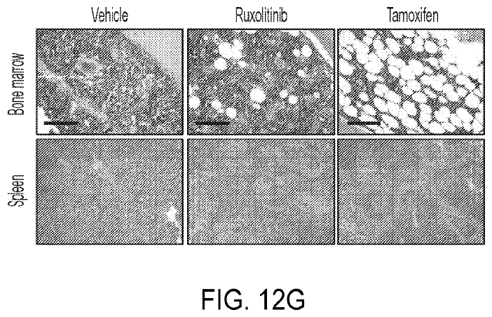

Given the limited ability of current JAK2 inhibitors to achieve disease modification and/or clonal remissions in polycythemia vera and myelofibrosis, the phenotypic and transcriptional effects of JAK inhibitor therapy with ruxolitinib were compared to the effects of Jak2^{V617F} reversal. Similar RNA-seq was performed on Jak2^{V617F} LSKs and MEPs following 7 days of ruxolitinib treatment (n=3) and compared to the effects of Jak2^{V617F} deletion (n=3). Analysis revealed that JAK-STAT target gene expression and erythroid pathway gene expression were much less potently inhibited with ruxolitinib than with Jak2^{V617F} deletion. Specifically, while similar reduced expression of negative regulators of JAK/STAT signaling (e.g. Socs2/3, Pim2, Cish) with ruxolitinib treatment were observed, the degree of down-regulation of STAT5 target gene expression was less pronounced as compared to Jak2^{V617F} deletion (RUX NES −0.913, p=0.84 vs. TAM NES −1.51, p=0.003) (FIG. 4A, FIG. 12A). Furthermore, the alterations in erythroid pathway gene expression (NES=1.45, p=0.012 vs. NES-1.82, p=0.0005) and myeloid progenitor frequencies observed with Jak2^{V617F} deletion were not observed with ruxolitinib (GMP: VEH 6.93% vs. RUX 6.66% vs. TAM 20.1%, p=0.91 vs. p<0.0001, MEP: VEH 27.1% vs. RUX 35.3% vs. TAM 14.2%, p=0.25 vs. p=0.014), a finding similarly observed when cells were cultured over BMECs (FIGS. 4B-4C, FIG. 12B). Further, expression of the gene sets associated with TGFb (p=0.65), and TNF/NFkB, (p=0.90) inflammatory signaling pathways were also not significantly altered with ruxolitinib treatment, in contrast to Jak2^{V617F} deletion. This lack of pro-inflammatory signaling response to ruxolitinib was consistent with human data showing increased NFkB accessibility in the setting of ruxolitinib therapy (FIG. 4D, FIG. 12C).

To evaluate the phenotypic effects of Jak2^{V617F} deletion in direct comparison to JAK kinase inhibition, an in vivo trial lasting 6 weeks comparing ruxolitinib to Jak2^{V617F} deletion was performed (FIG. 12D). A greater improvement in hematologic parameters, spleen weights (mean VEH 457 mg vs. RUX 235 mg vs. TAM 125 mg, p=0.0027), and restoration of histopathologic morphology in both bone marrow and spleen was observed, as well as reductions in fitness by CD45.2 chimerism in peripheral blood of Jak2^{V617F}-deleted mice versus ruxolitinib treated mice (VEH 40.7% vs. RUX 37.7% vs. TAM 17.3%, p=0.0059) (FIGS. 4E-4F, FIGS. 12E-12G). Most importantly, the reduction in mutant cell fraction seen with Jak2^{V617F} deletion, including in hematopoietic progenitor (GMP p<0.0001, MEP p<0.0001) and LSK stem-cell enriched populations (mean VEH 87.9% vs. RUX 87.6% vs. TAM 28.7%, p<0.0001), was not observed with pharmacologic JAK2 inhibition (FIG. 4G). It was previously shown that the type II JAK inhibitor CHZ868 showed improved efficacy compared to ruxolitinib in vivo. Consistent with these observations, treatment with CHZ868 showed greater efficacy than ruxolitinib in regard to improvement in hematologic parameters (Hct: CHZ868 50.3% vs. RUX 85.8%, p<0.0001) and spleen volume reduction (CHZ 76 mg vs. RUX 235 mg, p<0.0001), on par with Jak2^{V617F} deletion. Significant reductions in MEP, GMP, and LSK mutant allele burden were also observed in CHZ868 treated mice compared to vehicle/ruxolitinib-treated mice (LSK: p=0.02, GMP: p=0.013, MEP: p=0.013), but not to the extent seen with Jak2^{V617F} deletion (FIGS. 4E-4G). These data indicate that current JAK inhibitors do not sufficiently inhibit mutant JAK2 signaling and that more potent target inhibition offers the potential for greater therapeutic efficacy.

Recent studies have suggested that MAPK signaling has an important role in MPN disease cell survival in the setting of type I JAK inhibitor therapy, and recent work has implicated the MAPK-dependent factor YBX1 as a critical mediator of JAK2^{V617F}-mutant cell persistence. Here, distinct effects on MAPK activity by RNA-Seq with ruxolitinib treatment vs. Jak2^{V617F} deletion in comparison to vehicle treated mice were observed, with MAPK activation in response to ruxolitinib (NES −1.65, padj=0.0003) and reduced MAPK output with Jak2^{V617F} deletion (NES 1.35, padj=0.043) (FIG. 4H). Immunohistochemistry of bone marrow sections confirmed increased phospho-ERK abundance in ruxolitinib-treated mice that was abrogated with Jak2^{V617F} deletion (FIG. 4I), and expression of Ybx1 in sorted cKit+ cells was increased with ruxolitinib therapy and suppressed with Jak2^{V617F} deletion (Rel. exp VEH 1.37 vs. RUX 2.52 vs. TAM 0.42, p=0.0094) (FIG. 4J). These data suggest that potent, mutant-specific Jak2^{V617F} targeting can abrogate pathologic MAPK signaling and Ybx1-mediated persistence of Jak2^{V617F} mutant HSPCs.

### Example 4: Jak2^{V617F} Dependency with Cooperative TET2^{f/f} Loss

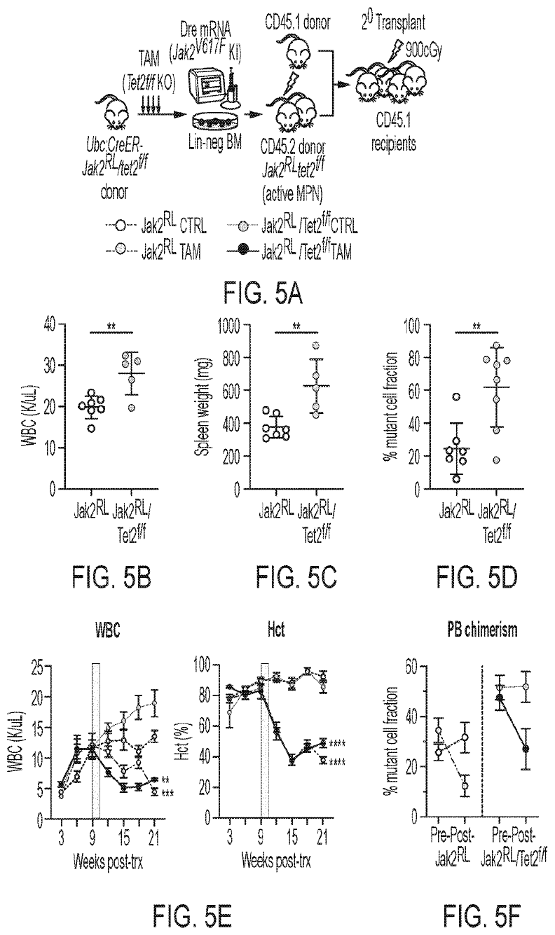

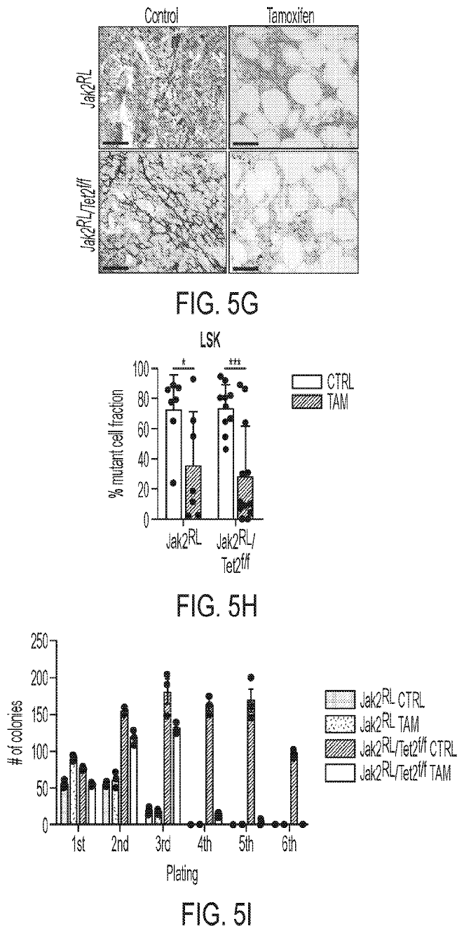

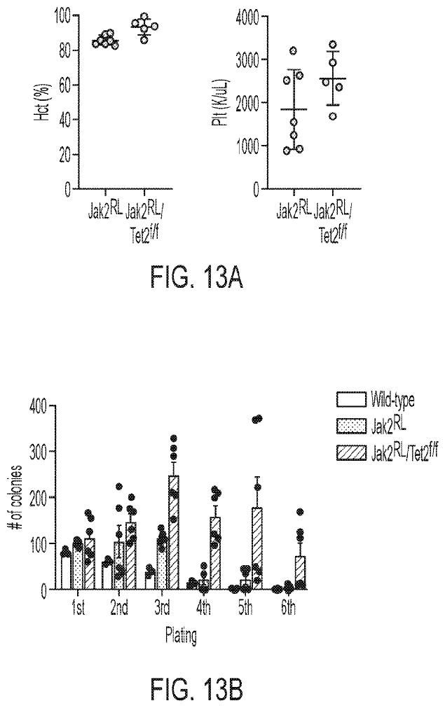

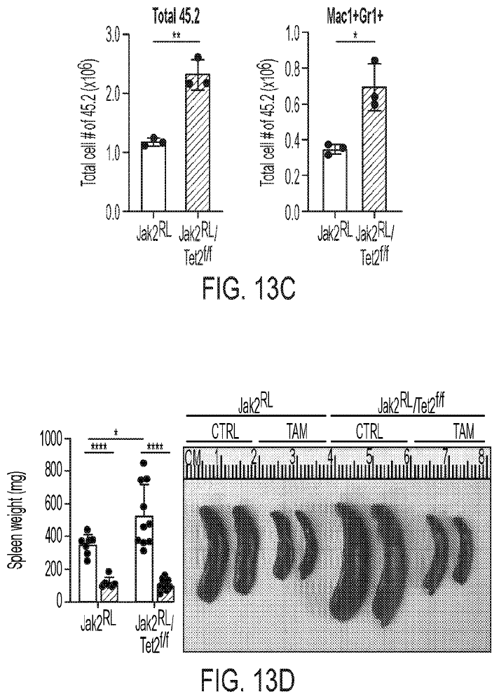

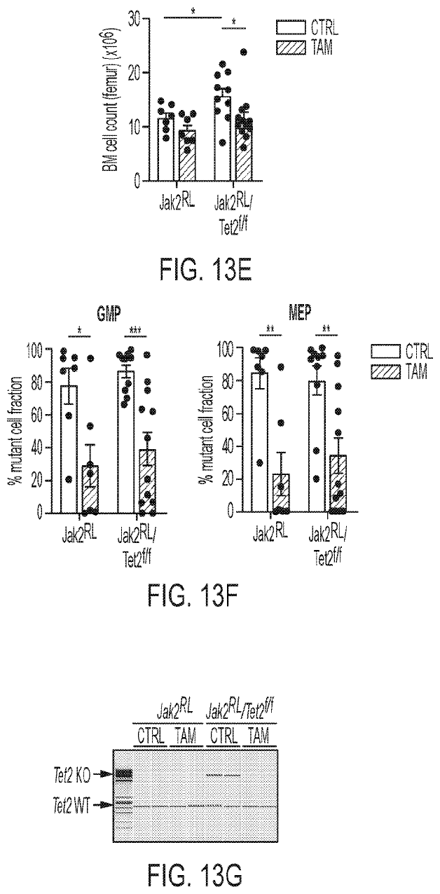

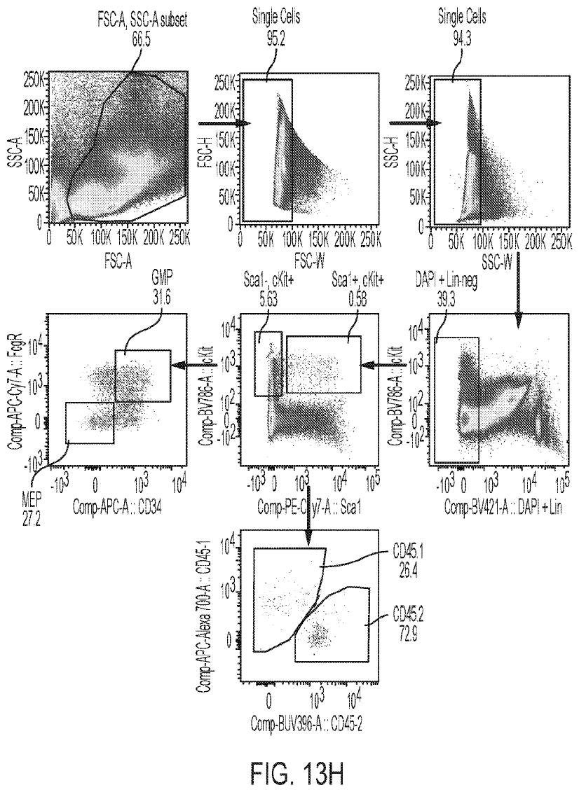

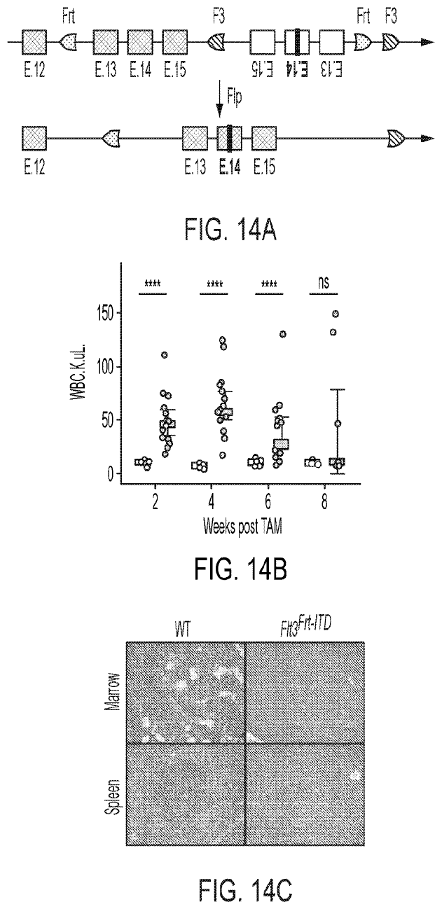

Previous studies of mutational order in primary MPN cells have shown that cooperating mutations in epigenetic regulators, including TET2, can precede the acquisition of JAK2^{V617F} and that antecedent TET2 mutations can alter the in vitro sensitivity to ruxolitinib. In addition, in vitro/in vivo studies have shown that concurrent TET2 and JAK2^{V617F} mutations promote enhanced mutant HSC fitness and increased risk of disease progression. The Jak2^{RL} system allows for the assessment of Jak2^{V617F} dependency in the setting of co-occurring mutant allele activation/inactivation, including in the context of antecedent mutations in epigenetic regulators. Therefore, the impact of Jak2^{V617F} activation in concert with pre-existing Tet2 loss with the reversible Jak2^{RL} allele was assessed (FIG. 5A). Mice transplanted with Ubc:CreER-Jak2^{V617F}/Tet2^{f/f} cells demonstrated enhanced leukocytosis (mean 26.79 K/uL vs. 19.97 K/uL, p=0.008), increased spleen volumes (mean 626 mg vs. 357 mg, p=0.0047) and fibrosis, expanded peripheral blood chimerism (mean 61.9% vs. 24.9%, p=0.0042), and improved serial replating capacity in colony forming assays compared to Ubc:CreER-Jak2^{V617F} transplanted mice (FIG. 5B-5D, FIGS. 13A-13B). These data are consistent with previous Jak2^{V617F}/Tet2^{f/f} models and highlight the utility of the Dre-Cre dual recombinase system to model evolution from clonal hematopoiesis (CH) to overt MPN. Ex vivo co-culture of Jak2^{V617F}/Tet2^{f/f} cells over BMECs exhibited a near 2-fold increase in hematopoietic cell output (2.32×10⁶/mL vs. 1.18×10⁶/mL, p=0.0017), particularly among Mac1+ mature myeloid cells (0.69×10⁶/mL vs. 0.35×10⁶/mL, p=0.011), compared to Jak2^{V617F} cells consistent with the known role of Tet2 loss-of-function in enhancing myeloid lineage commitment (FIG. 13C). This in vitro expansion was further reduced with 40HT treatment in comparison to ruxolitinib suggesting double-mutant cells maintain reliance on JAK/STAT signaling to promote aberrant cell growth in vitro.

The effects of Jak2^{V617F} deletion on Jak2^{V617F}/Tet2^{f/f} mutant cell fitness in vivo in competition with CD45.1 marrow were evaluated. Treatment with tamoxifen at 9 weeks post-transplant resulted in normalization of hematologic parameters (p<0.005) and reductions in peripheral blood mutant cell fraction of double-mutant cells to a similar extent observed with Jak2^{V617F} deletion in single-mutant Jak2^{RL} transplanted mice (FIG. 5E-5F). Further, spleen sizes and total BM cellularity were also normalized, and while the extent of reticulin fibrosis was increased in Jak2^{V617F}/Tet2^{f/f} compared to Jak2^{V617F} deletion resolved fibrosis in both mutational contexts (FIG. 5G, FIGS. 13D-13E). The reduction in mutant cell fraction, as was observed with single-mutant mice, persisted down to the level of HSPCs in TAM-treated Jak2^{V617F}/Tet2^{f/f} mice, including within the LSK stem cell-enriched compartment (FIG. 5H, FIG. 13F). Furthermore, in a subset of mice, Tet2^{f/f}knock-out bands in whole marrow at time of sacrifice were unable to be detected, and cells harvested from Jak2^{V617F}/Tet2^{f/f} recipient mice following oncogenic deletion were unable to serially replate indicating loss of self-renewal capacity in comparison to control double-mutant mice (FIG. 5I, FIG. 13G). These data support the notion that co-occurring mutations in Tet2 do not dramatically alter reliance on JAK/STAT signaling for disease maintenance, and that despite the fitness advantage engendered by Tet2 loss on hematopoietic stem cells, the alterations in HSC fitness in the setting of mutant Jak2^{V617F} reversion suggest a unique dependency on oncogenic Jak2^{V617F} that renders double-mutant cells susceptible to eradication.

Mutated kinases occur frequently in cancer and are amenable to targeted inhibition; however, mechanisms which mediate acquired resistance have been observed for most targeted therapies. By contrast, current JAK inhibitors fail to eliminate Jak2^{V617F}-mutant clones in MPN suggesting inadequate target inhibition and/or other genetic/non--genetic factors mediate Jak2^{V617F}-mutant cell persistence in the setting of JA K inhibitor therapy. Here, preclinical models show that there is an absolute requirement for Jak2^{V617F} in MPN cells and that mutant-specific targeting of Jak2^{V617F} abrogates MPN features, reduces mutant cell fraction, and extends overall survival with concomitant depletion of disease-sustaining stem cells within the HSPC compartment. Further, the data suggest that Jak2^{V617F} dependency persists even in the setting of antecedent mutations in epigenetic regulators. These data support the notion that improved targeting of JAK2 signaling and downstream effectors offers greater therapeutic potential than current JAK kinase inhibitors, and Jak2^{V617F}-mutant-selective inhibition represents a potential curative strategy for the treatment of MPN patients. Moreover, the feasibility of the dual-recombinase system to evaluate oncogenic signaling dependencies in vivo has been demonstrated, and a similar approach will allow for the assessment of oncogenic dependencies and mechanisms of mutant-mediated transformation across a spectrum of malignant contexts.

### Example 5: Methods for Examples 1-4

Experimental animals. All animal experiments were performed in accordance with the Memorial Sloan Kettering Cancer Center (MSKCC) Institutional Animal Care and Use Committee-approved animal protocol. Animal care was in strict compliance with institutional guidelines established by MSKCC and the Guide for the Care and Use of Laboratory Animals (National Academy of Sciences 1996). Mice with an intact immune system were used for all experiments. All animals were drug and test naïve and not involved in previous procedures, maintained on a 12 hour light-dark cycle, and with access to water and standard chow ad libitum. Veterinary staff conducted regular husbandry procedures and provided routine care and monitoring. Animals were monitored daily for signs of disease or morbidity, failure to thrive, weight loss, open skin lesions, bleeding, infection, or fatigue and sacrificed immediately if they exhibited any signs of the above. Mice harboring the Jak2^{RL} allele were generated by Ingenious Targeting Laboratory (Ronkonoma, NY) in a C57BL/6 background. Conditional Jak2^{V617F} knock-in mice, RC::RLTG reporter mice, Tet2^{f/f} conditional knock-out mice, Cre- TdTomato reporter mice, and UbcCreER+ mice have been described previously.^{6,8,17,24} 6-8 week old female and male Jak2^{RL} or Jak2^{RL}/Tet2^{f/f} donor mice were used for Dre electroporation knock-in experiments. Age-matched 6-10 week old female mice were used as donors for all transplant experiments (Ly5.1 CD45.1 competitive or C57BL/6J non-competitive).

### Mouse Genotyping.

DNA was isolated using the DNeasy Blood & Tissue Kit (Qiagen, Germantown, MD). Jak2^{V617F} knock-in genotyping was carried out with the following primers: FWD: 5′-GCCATCTTTCCAGCCTAAAATTAG-3′ (SEQ ID NO: 83); REV: 5′-TCCAAAGAGTCTGTAAGTACAGAACT-3′ (SEQ ID NO: 84) and with the following reaction conditions: 94° C. for 3 minutes followed by 15 cycles of 94° C. for 15s, 65° C. for 15s, and 72° C. for 30s decreasing by 1° C. per cycle, and then followed by an additional 25 cycles of 94° C. for 15s, 50° C. for 15s, and 72° C. for 30s. Jak2^{V617F} knock-out genotyping was carried out using the following primers: FWD: 5′-GCCATCTTTCCAGCCTAAAATTAG-3′ (SEQ ID NO: 83); REV: 5′-ACCAGTTGCTCCAGGGTTACACG-3′ (SEQ ID NO: 85) and with the following reaction conditions: 94° C. for 2 minutes followed by 30 cycles of 94° C. for 30s, 53° C. for 30s, and 72° C. for 30s. Sequencing of the unrecombined Rox-lox locus was carried out using the following primers: FWD: 5′-AGGAGCATCGATGACTACATGATGAG-3′ (SEQ ID NO: 86); REV: 5′-AGACTCTCCACGGTCTCATCTACG-3′ (SEQ ID NO: 87) and with the following reaction conditions: 98° C. for 30 seconds followed by 35 cycles of 98° C. for 10s, 65° C. for 15s, and 72° C. for 30s. Tet2 primers were carried out using the following primers/conditions: FWD: 5′-AAGAATTGCTACAGGCCTGC-3′ (SEQ ID NO: 88); REV: 5′-TTCTTTAGCCCTTGCTGAGC-3′ (SEQ ID NO: 89); ExR: 5′-TAGAGGGAGGGGGCATAAGT-3′ (SEQ ID NO: 90) and with the following reaction conditions: 94° C. for 2 minutes followed by 39 cycles of 94° C. for 35s, 58° C. for 45s, and 72° C. for 55s. Annotation of PCR genotyping results was carried out on a QIAxcel Advanced System (Qiagen) and analyzed using QIAxcel ScreenGel software (Qiagen).

Dre mRNA Electroporation.

Dre mRNA was purchased from TriLink Biotechnologies (San Diego, CA) and electroporation carried out using the Neon Transfection System (ThermoScientific) per the manufacturer's protocol. Specifically, bone marrow donor cells were isolated from limb bones into PBS (pH 7.2) containing 2% fetal calf serum via centrifugation. After red blood cell lysis, single-cell suspensions were depleted of lineage-committed hematopoietic cells using a Lineage Cell Depletion Kit according to manufacturer's protocol (EasySep™, StemCell Technologies, Inc.). 2.5-3.0×10⁶ lineage-depleted bone marrow was then washed in PBS and then resuspended in 135 uL Buffer T to which 15 μL of Dre mRNA (at 1 μg/μL) was quickly added and electroporated at the following conditions: 1700V for 20 ms×1 pulse. The cells were then pipetted into penicillin-streptomycin free StemSpan SFEM medium with thrombopoietin (TPO; 20 ng/mL; PeproTech) and stem cell factor (SCF; 20 ng/mL; PeproTech), cultured for two hours, and then subsequently harvested and washed/resuspended in phosphate buffered saline (PBS) and transplanted via lateral tail vein injection into lethally irradiated (900cGy) 6-8 week old C57BL/6J recipient mice at approximately 4×10⁵ cells per recipient along with 50,000 un-electroporated whole bone marrow support cells. Double-mutant Jak2^{RL}/Tet2^{f/f} electroporations were carried out as above, except donor mice were dosed with tamoxifen (100 mg/kg by oral gavage daily ×4; purchased from MedChemExpress) 6-8 weeks prior to harvest, and excision confirmed prior to Dre electroporation.

### Transplantation Assays and In Vivo Experiments.

Jak2^{RL} and Jak2^{RL}/Tet2^{f/f} lines were crossed to UbcCreER tamoxifen-inducible Cre lines and RLTG dual-recombinase reporter lines.^{17,24} Primary recipient mice transplanted with Dre mRNA-recombined UbcCreER-Jak2^{RL} or UbcCreER-Jak2^{RL}/Tet2^{f/f} bone marrow cells were bled every 3-4 weeks post-transplant to monitor disease status. Peripheral blood was isolated by submandibular bleeds. Complete blood counts were assessed on a ProCyte Dx (IDEXX Laboratories, Westbrook, ME) per manufacturer's instruction. For competitive repopulation assays, 1.2×10⁶ whole bone marrow from primary transplant recipient mice exhibiting MPN was harvested 6-8 weeks post-transplant and combined with age-matched 0.8×10⁶ CD45.1 (Jackson Laboratories, Bar Harbor, ME) whole bone marrow and transplanted into 6-8 week old lethally irradiated CD45.1 secondary recipient mice. To induce Cre and delete Jak2^{V617F}, mice were treated with tamoxifen (TAM; purchased from MedChemExpress) 100 mg/kg daily (dissolved in corn oil) by oral gavage ×4 followed by 14 days of TAM chow approx. 80 mg/kg daily (ENVIGO). Tamoxifen control studies were carried out using similar dosing schedules on 45.1 mice transplanted in competition with Dre-electroporated, Cre-negative Jak2^{RL} MPN bone marrow cells. For terminal tissue isolation, mice were euthanized with CO₂ asphyxiation, and tissues were dissected and fixed with 4% paraformaldehyde for histopathological analysis. For whole bone marrow isolation, the femurs, hips, and tibias were dissected and cleaned. Cells were isolated by centrifugation at 8000×G for 1 minute followed by RBC lysis (BioLegend). Bone marrow cell numbers were determined via an automatic cell counter (ViCell Blu, Beckman Coulter). Single cell suspensions were generated from crushed whole spleen and filtered through a 70 uM filter. RBC lysis was performed (BioLegend) and cells were prepared for downstream processing or frozen.

### In Vivo Drug Studies.

For in vivo inhibitor studies, approximately 8 weeks following transplant, secondary transplant cohorts of lethally-irradiated mice transplanted with UbcCreER-Jak2^{RL} bone marrow in competition with CD45.1 marrow (as above) and exhibiting active MPN were bled and cohorted based on peripheral blood CD45.2 chimerism and total WBC count to achieve congruency across treatment arms. Mice were then treated with ruxolitinib (60 mg/kg P.O. twice daily; dissolved in 20% Captisol in PBS; purchased from MedChemExpress), CHZ868 (30 mg/kg P.O. daily; dissolved in 0.5% methylcellulose +0.5% Tween-80 in dH2O; purchased from MedChemExpress), tamoxifen to delete Jak2^{V617F} (as above; purchased from MedChemExpress), or vehicle. Investigators were not blinded to the identity of mice or samples. Mice were treated for a total of 6 weeks before timed sacrifice and marrow/spleen harvested as above.

### Bone Marrow Endothelial Cell (BMEC) Culture.

Bone marrow cells were isolated from limb bones into FACS buffer (phosphate buffered saline (PBS)+2% fetal bovine serum) via centrifugation. After red blood cell lysis, single-cell suspensions were depleted of lineage-committed hematopoietic cells using a Lineage Cell Depletion Kit according to manufacturer's protocol (EasySep™, StemCell Technologies, Inc.). Subsequently, 50,000 of the resulting lineage⁻ cells were plated on a confluent monolayer of BMECs in a single well of a 12-well plate. Each well had 1 mL StemSpan SFEM (StemCell Technologies, Inc.) with 20 ng/mL recombinant murine SCF (PeproTech) in addition to the corresponding drug treatment: either 4--hydroxytamoxifen (4-OHT; Sigma Aldrich; stock concentration:13 mM) or its vehicle, appropriately diluted in media to its final concentration (i.e., 0.01% (v/v) of ethanol, or 200 nM, 400 nM or 1 uM of 4-OHT) (three replicates/condition). The BMECs were seeded two days before plating the lineage- cells at a density of 100,000 cells/well. Co-cultures were maintained for a total of 7 days at 37° C. and 5% CO₂, with media being completely refreshed with the original SCF and drug/vehicle concentrations. 4OHT or ethanol EtOH vehicle as added to the culture on day 1 and again on day 4. On day 7, total cells were harvested with Accutase (Biolegend) and cell numbers were determined via an automatic cell counter (ViCell Blu, Beckman Coulter). Cells were then stained with the desired antibody cocktail and phenotyped by flow cytometry.

### Flow Cytometry and Western Blot.

Following single cell preparation, murine peripheral blood, whole bone marrow, or spleen mononuclear cells were lysed for 10 minutes with RBC lysis buffer (BioLegend, San Diego, CA) and washed twice with FACS buffer (phosphate buffered saline (PBS)+2% fetal bovine serum). Cells were then resuspended in Fe (CD16/32) block for 15 minutes and then subsequently stained with a cocktail comprised of antibodies targeting CD3 (17A2), CD45R/B220 (RA3-6B2), Gr-1 (RB6-8C5), CD11b (M1/70), CD45.2 (104), and CD45.1 (A20) for 30 minutes. For hematopoietic stem/progenitor cell analysis, lysed bone marrow was stained with a cocktail of lineage markers along with antibodies against c-Kit (2B8), Sca-1 (D7), FcγRII/III (2.4G2), CD34 (RAM34), CD150 (9D1), CD105, CD71 (R17217), CD41 (MWReg30), and CD48 (HM48-1). Erythroid progenitor flow was carried out on unlysed bone marrow with the following antibodies: All FACS antibodies were purchased from BD, BioLegend, or eBioscience. Following antibody incubation, cells were washed with FACS buffer and resuspended in a DAPI-containing FACS buffer solution for analysis and sorting. Samples were run on a Fortessa (Becton Dickinson) using FACSDiva software and analyzed with FlowJo (Treestar, Ashland, OR, USA). For Western blot analysis, whole-cell protein extracts from harvested splenocytes were prepared using RIPA buffer (ThermoScientific, Rockford, IL) containing a protease/phosphatase inhibitor cocktail (Thermo Scientific). Protein quantification was performed using the Pierce BCA protein assay kit (ThermoScientific) and analyzed on a Cytation 3 plate reader (BioTek). Proteins were separated by NuPAGE 4-12% Bis-Tris Gel and transferred to a nitrocellulose membrane. The following antibodies were used: β-actin (Cell Signaling 4970S), STAT5 (Cell Signaling 94205S), and pSTAT5 (Cell Signaling, 9359S). Images were obtained using the ChemiDoc Imaging System (BioRad) and analyzed using ImageLab software (BioRad).

### Histology Staining and Immunohistochemistry, Photography.

Tibia and spleen samples were fixed in 4% paraformaldehyde for over 24 hours and then embedded in paraffin. Paraffin sections were cut on a rotary microtome (Mikrom International AG), mounted on microscope slides (ThermoScientific), and air-dried in an oven at 37° C. overnight. After drying, tissue section slides were processed either automatically for hematoxylin and eosin (H&E) staining (COT20 stainer, Medite), or manually for reticulin staining. The following antibodies were used for immunohistochemistry: MacI (Cedarlane CL8942B, 1:100), Ter119 (BDBioscience, 550565 1:200), and phospho-ERK (Cell Signaling 4376, 1:100). Pictures were taken at 100×, 200× and 400× (H&E and reticulin) magnification using an Olympus microscope and analyzed with Olympus Cellsens software. Tissue sections were formally evaluated by a hematopathologist (W. Xiao), including reticulin scoring.

### Assessment of Cell Growth, Apoptosis, and Viability.

Apoptosis was measured by flow cytometry on a Fortessa (Becton Dickinson) cytometer with Annexin V PerCPCy5.5 antibody (BioLegend) and Annexin binding buffer (BioLegend) at 1:50 dilution in combination with DAPI as live/dead cell stain.

### Colony Forming Assays.

To assess colony formation and serial replating capacity, RBC lysed 50,000 whole bone marrow cells were seeded in 1.5 mL MethoCult M3434 (Stem Cell Technologies) in triplicate on 6 well plates and scored on day 8. For replating, cells were harvested and pooled and then re-seeded once more at 50,000 cells/well in 1.5 mL MethoCult M3434 in triplicate.

### Serum Cytokine Profiling.

Serum samples were diluted two-fold with PBS (pH 7.2) and stored at −80° C. until analysis. Cytokine levels were measured in duplicates (62.5 uL each) by Eve Technologies Corporation (Mouse Cytokine Array/Chemokine Array 44-Plex, Calgary, AB, Canada).

### RNA-Seq and Data Analysis.

For gene expression analysis, secondary cohorts of mice transplanted with UbcCreER-Jak2^{RL} bone marrow 8 weeks post-transplant and exhibiting MPN were treated with ruxolitinib (60 ng/g P.O. BID), tamoxifen (100 mg/kg daily ×4 followed by 80 mg/kg daily of TAM chow ×3) for 7 days and then sacrificed along with an untreated MPN control cohort. Lineage-depleted marrow was isolated and then TdTonmato+ (Jak2^{RL} knock-in) or GFP+ (Jak2^{RL} knock-out) LSKs and MEPs were sorted on a FACSAria III directly into Trizol LS (Invitrogen) and stored at −80° C. until processing. RNA was subsequently isolated using the Direct-Zol Microprep Kit (Zymo Research, R2061) according to manufacturer's protocol and quantified using the Agilent High Sensitivity RNA ScreenTape (Agilent 5067-5579) on an Agilent 2200 TapeStation. cDNA was generated from 1 ng of input RNA using the SMART-Seq HT Kit (Takara 634455) at half reaction volume followed by Nextera XT (Illumina FC-131-1024) library preparation. cDNA and tagmented libraries were quantified using High Sensitivity D5000 ScreenTape (5067-5592) and High Sensitivity D1000 ScreenTape respectively (5067-5584). Libraries were sequenced on a NovaSeq at the Integrated Genomics Operation (IGO) at MSKCC.

### ATAC-Seq and Data Analysis.

Chromatin accessibility assays utilizing the bacterial Tn5 transposase were performed as described. Briefly, 5.0×10⁴ sorted reporter-positive TdTomato+ (Jak2^{RL} knock-in) or GFP+ (Jak2^{RL} knock-out) cKit+ bone marrow cells from mice treated for 7 days with ruxolitinib (60 mg/kg P.O BID), tamoxifen, or an untreated MPN control cohort were sorted on a FACSAria III into PBS and subsequently lysed and incubated with transposition reaction mix containing PBS, Tagment DNA buffer, 1% Digitonin, 10% Tween-20, and Transposase (Illumina). Samples were then incubated for 30 minutes at 37° C. in a thermomixer at 1000 rpm. Prior to amplification, samples were concentrated with the DNA Clean and Concentrator Kit-5 (Zymo). Samples were eluted in 20 μL of elution buffer and PCR-amplified using the NEBNext 2× Master Mix (NEB) for 11 cycles and sequenced on a NextSeq 500 (Illumina). Libraries were sequenced on a NovaSeq at the Integrated Genomics Operation (IGO) at MSKCC.

### Quantitative Real-Time PCR.

Total RNA was extracted from magnetic-bead isolated cKit+ bone marrow (Miltenyi Biotec) using the Direct-zol RNA extraction kit (Zymo) per manufacturers' protocols respectively. Complementary DNA was then reverse transcribed using the Verso cDNA Synthesis kit (ThermoFisher Scientific). Ybx1 expression was evaluated by quantitative reverse-transcription (qRT) PCR using Taqman probes purchased from ThermoFisher (Mm00850878_g1) on the RealPlex thermocycler (ThermoFisher Scientific, Fairlawn, NJ).

### Statistical Analysis.

Statistical analyses were performed using Student's t-test (normal distribution) using GraphPad Prism version 6.Oh (GraphPad Software, San Diego, CA). Kaplan-Meier curves were determined using the log-rank test. P<0.05 was considered statistically significant. The number of animals, cells and experimental replication can be found in the respective figure legends. FASTQ files were demultiplexed using a java script from Takara. FASTQ files were mapped and transcript counts were enumerated using STAR (genome version mm10 and transcript version ensembl XX). Counts were input into R and RNA-sequencing analysis using DESeq2. Gene set enrichment analysis was performed using the fgsea package with genesets extracted from the msigdbr package. Single sample gene set enrichment analysis was performed using the gsva package. Figures were generated using ggplot2 and tidyhetamps packages.

### Example 6: Inducible Mouse Model of Flt3 Internal Tandem Duplication

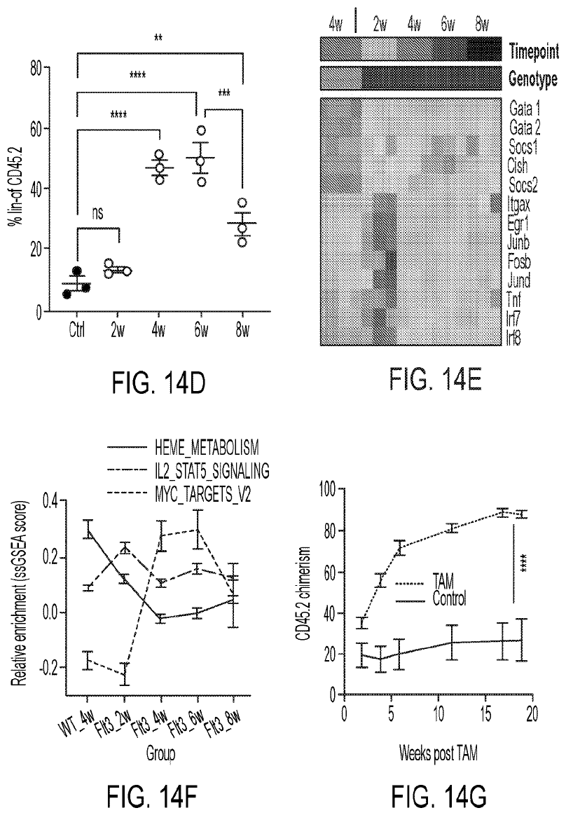

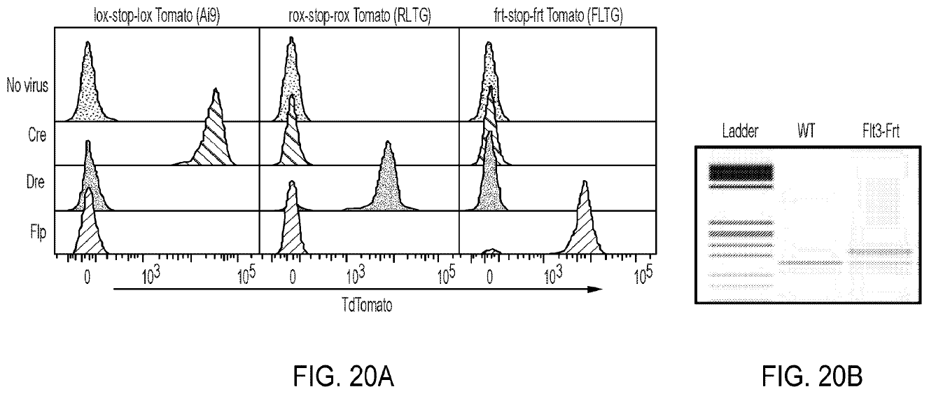

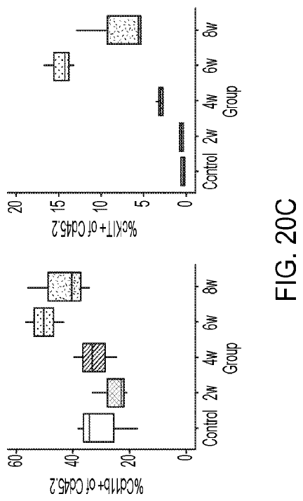

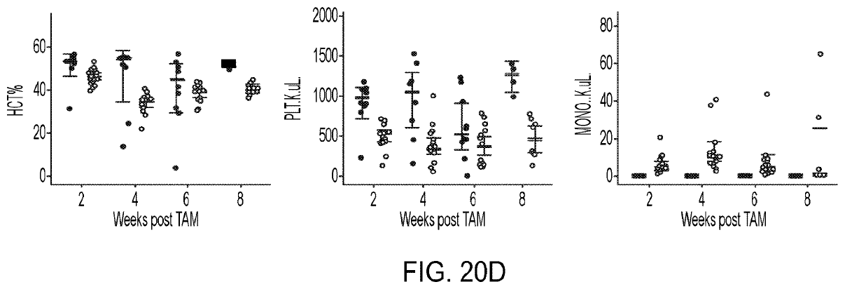

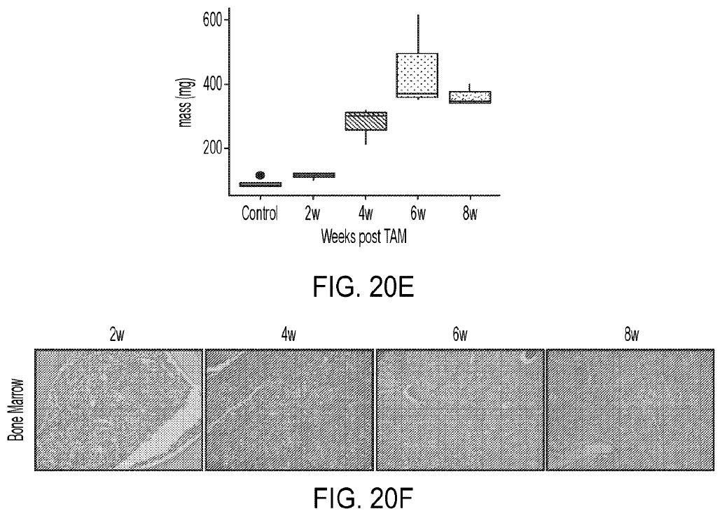

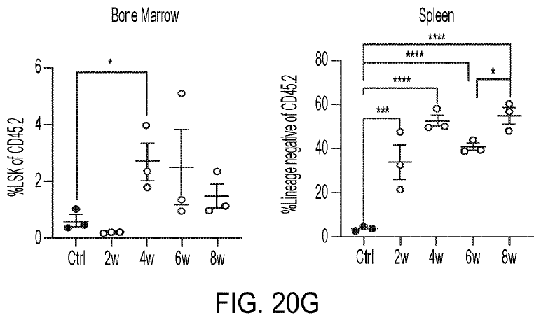

In order to model Flt3 mutations as late hits in leukemogenesis, an endogenously targeted, Flp-inducible Fft3^{Frt-ITD} allele which could be inverted by a tamoxifen (TAM) inducible FlpoERT2 allele was developed (FIG. 14A, FIG. 20B). By 6 weeks post tamoxifen administration, a pan-leukocytosis (mean WBC: Flt3^{Frt-ITD} 38.21, WT 10.37, p≤4.5×10⁻⁴) driven by a myeloid bias accompanied by a peak of cKIT+ cells in the blood was observed (mean±s.d. 6w:4.8±1.84% vs Control: 0.310±0.068% p≤5.27×10⁻³) (FIG. 14B, FIGS. 20B-20C). Critically, by 8-10 weeks, this pan leukocytosis had largely resolved, with WBCs decreasing to near normal levels (p≤0.133) despite persistent anemia (mean HCT %: 40.4 vs 51.4 p≤1.71E-07), and thrombocytopenia (mean PLT K/uL 467 vs 1240 p≤8.96E-06) (FIG. 20D). Changes in the peripheral blood were accompanied by an increase in spleen mass (mean: 361.99 mg vs. 90.35 mg p≤2.84×10⁻³), and myeloid infiltrate into both the spleen and liver (FIG. 20E). An increase in immature myeloid cells, reduction in megakaryocytes and near absence of erythroid cells was evident in the bone marrow while splenic architecture was disrupted, pathological findings which persisted even after resolution of the leukocytosis (FIG. 14C, FIG. 20F). Along this time course, an expansion of lineage negative hematopoietic stem and progenitor cells (HSPCs; mean±s.d 6w 50.07±8.65% vs Control 0.55±3.79%, p≤0.0016), specifically the lin⁻Sca-1⁺cKit⁺ (LSKs), in the bone marrow was observed (4w vs Control marrow: p≤0.0382) (FIG. 141), FIG. 20G).

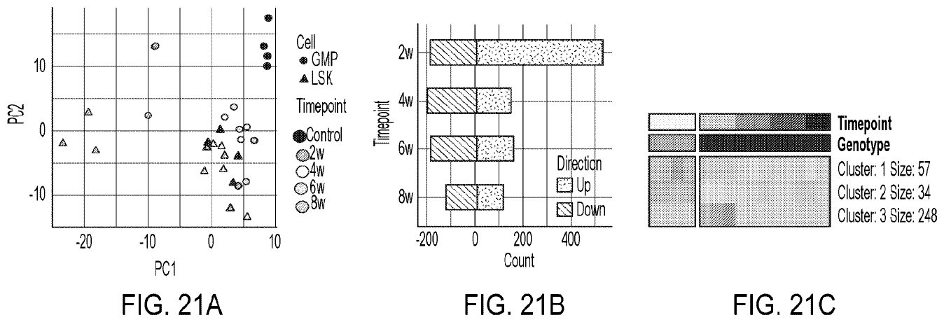

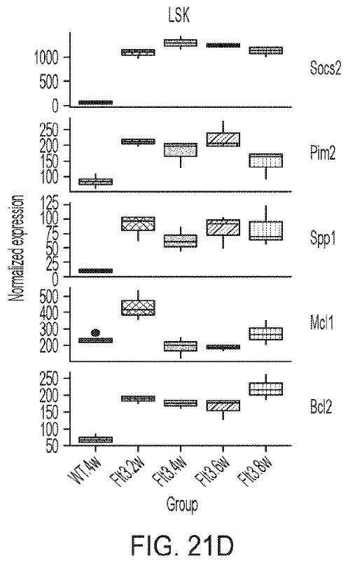

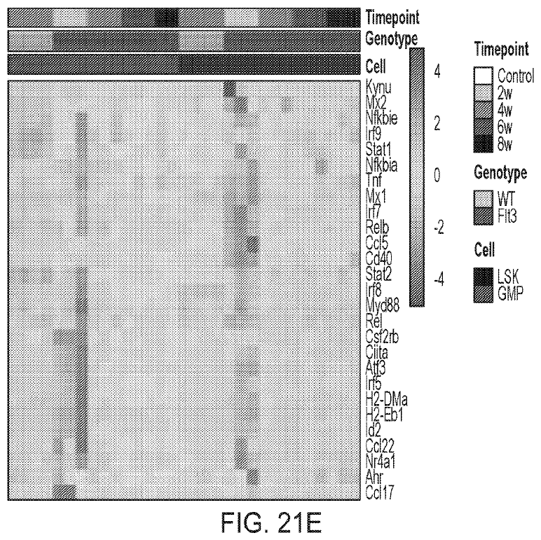

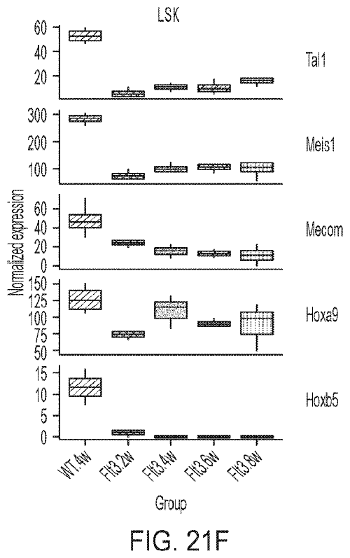

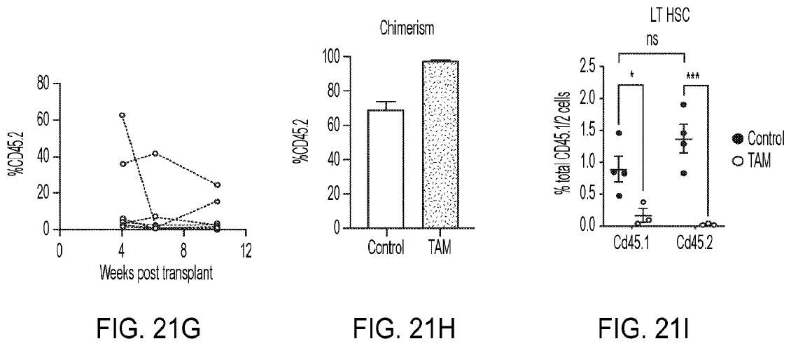

To determine how Flt3^{Frt-ITD} mutations changed the transcriptional landscape of HSPCs, low pass, multiplexed 3′ RNA-sequencing was performed on purified LSKs and GMPs from Flt3^{ITD} mice 24, 6 or 8 weeks after TAM administration, and from Flt3^{WT} mice 4 weeks after TAM (FIG. 21A). Along this timecourse >200 genes were downregulated in LSKs with Flt3^{Frt-ITD} activation, and over >500 were upregulated for at least one time point (FIG. 21B). These alterations could be largely consolidated into three distinct phases of transcriptional alterations along the 8 week time course including genes which were (1) acutely inactivated and downregulation was sustained (2) chronically activated, and (3) acutely dysregulated only at 2 weeks post Flt3^{ITD} activation (FIG. 21C). Sustained dysregulation was associated with a disengagement of heme synthesis typified by a downregulation of the critical erythroid transcription factors Gafa1 and Gafa2 (FIGS. 14E-14F) and activation of MYC target genes. Meanwhile, STAT5 transcriptional engagement appeared to peak at two weeks with subsequent sustained expression of negative feedback regulators including Cish, Socs2 and Socs3 (FIGS. 14E-14F. FIG. 21D). In addition to an inflammatory burst involving dendritic-cell like genes at 2 weeks, gene expression signatures indicative of KRAS signaling were also upregulated with transient expression of AP-1 complex members (Junb. Junb, Fos) and the MAPK target Egr1 (FIGS. 14E-14F, FIG. 21E). Critical regulators of normal and malignant self renewal including Tal1, Meis1, Mecom, Hoxb5 and Hoxa9 were downregulated throughout the time indicating alterations in the constituent populations of the heterogenous LSK pool (FIG. 21F).

To understand the functional consequences of these gene expression alterations on cellular fitness, competitive transplantation between Flt3^{Frt-ITD} and Cd45.1 WT cells was performed. Activation of Flt3^{Frt-ITD} with TAM 4 weeks after transplantation resulted in robust, sustained competitive outgrowth of Flt3^{Frt-ITD} compared to WT competitor cells (mean Cd45.2% at 18w 88.89% vs 25.8% p≤6.54×10⁻⁴). (FIG. 14G, FIG. 21G). Despite this out competition, Flt3^{Frt-ITD} mutant cells were incapable of robustly engrafting into recipient mice and propagating disease (FIG. 21H). In part this is likely due to a loss of long term hematopoietic stem cells (LT-HSCs), which was observed in both CD45.2 Flt3^{Frt-ITD} (Control: 1.37±0.44% vs 0.TAM 0.23±0.12%, p≤0.0004) and CD45.1 WT cells (mean±s.d.; Control: 0.9±0.4% vs TAM: 0.18±0.18, p≤0.0194) resolving a non-cell autonomous mechanism proposed in previous studies (FIG. 21I). These findings demonstrate that while Flt3^{Frt-ITD} cells can give rise to a robust myeloproliferative disease and maintain a competitive advantage over WT cells, they are incapable of serial transplantation and complete transformation to leukemia.

### Example 7: Cooperating Mutations License Mutant-Flt3 for Transformation

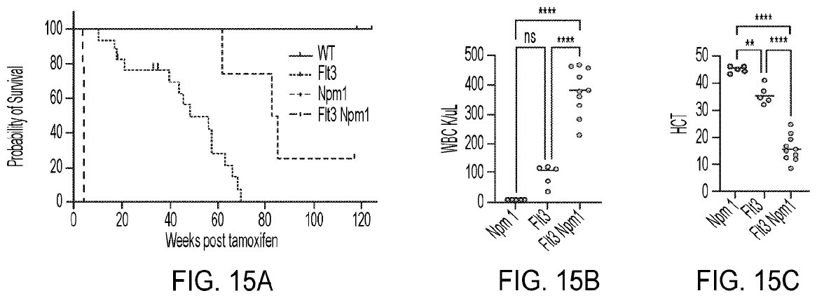

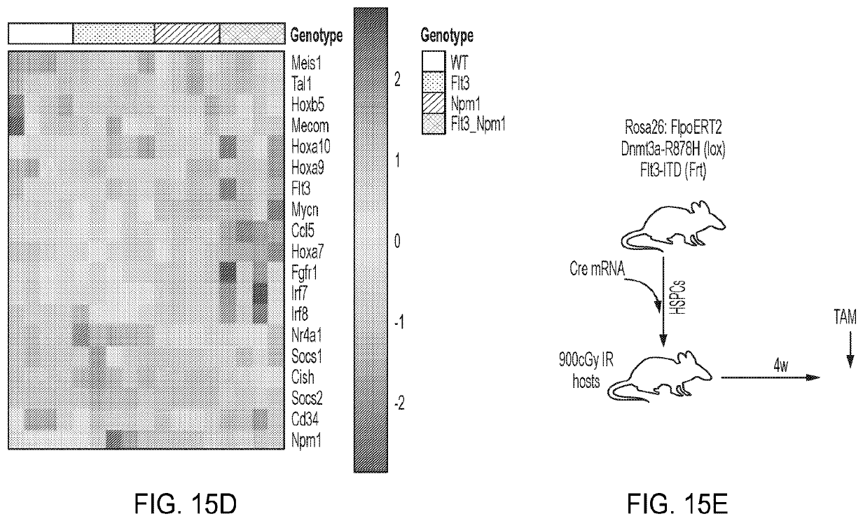

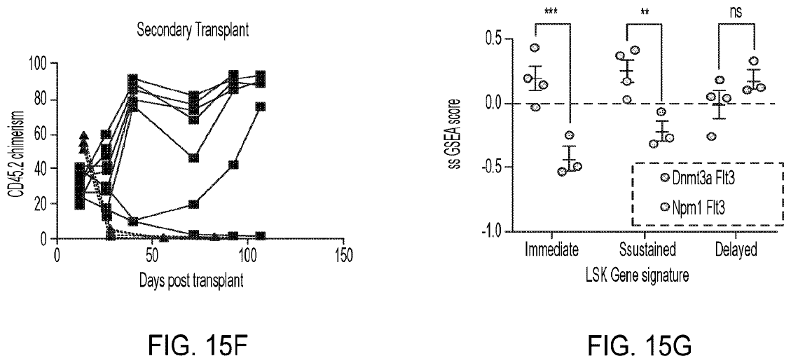

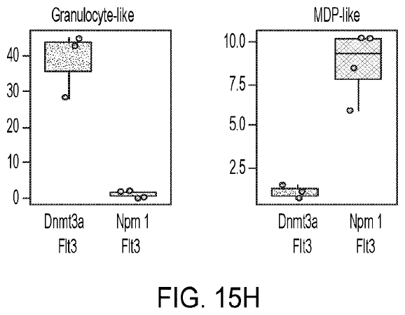

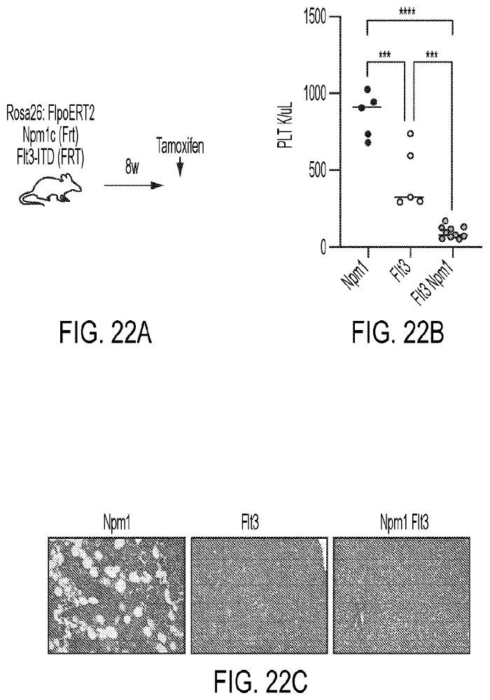

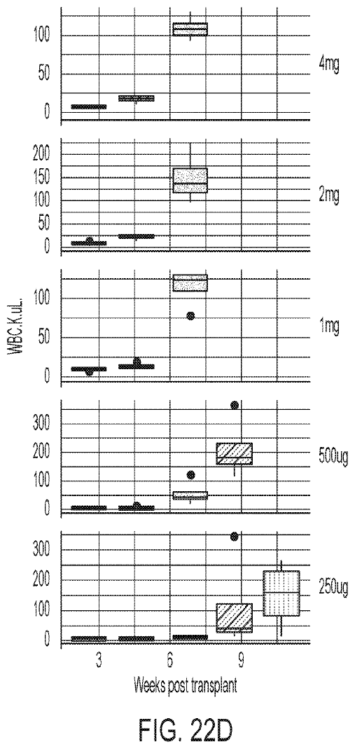

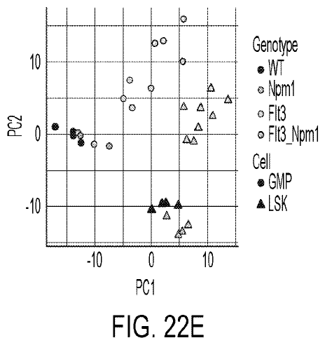

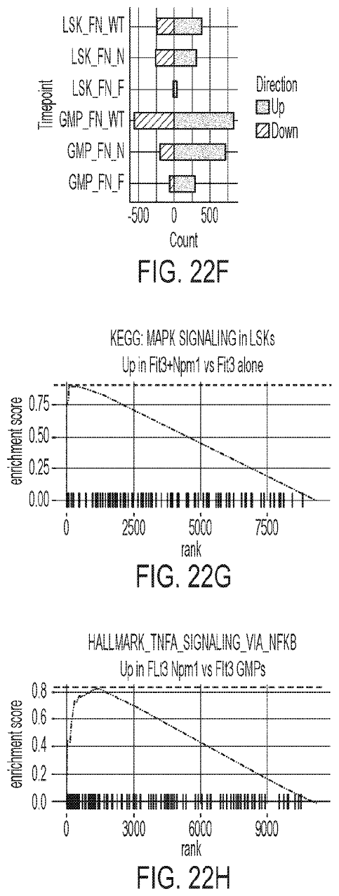

Flt3^{Frt-ITD} mice were crossed with a Npm1^{c-Frt} mutation for simultaneous activation with a TAM-inducible FlpoERT2. Primary Flt3^{Frt-ITD} mice showed a median survival 48 weeks, Npm1^{c-Frt} Flt3^{Frt-ITD} mice rapidly succumbed to disease within 5 weeks following TAM administration (FIG. 15A, FIG. 22A). Similar results were found in transplantation model where four weeks post TAM, Flt3^{Frt-ITD} Npm1^{c-Frt} mice exhibited a robust increase in WBC, severe anemia and thrombocytopenia compared to either Flt3^{Frt-ITD} or Npm1^{c-Frt} alone, with pathological features consistent with AML (FIGS. 15B-15C, FIGS. 22B-22C). Unlike Flt3^{Frt-ITD} single mutant mice, WBC in Flt3^{Frt-ITD} Npm1^{c-Frt} did not normalize, and displayed exponential expansion, even with limiting dilution of TAM (FIG. 22D). This apparent leukemic transformation was not associated with restored expression of Tall, Meis1, Mecom, or Hoxb5, rather Npm1^{c-Frt} Flt3^{Frt-ITD} LSKs possessed increased expression of Hoxa7, Hoxa9, and Hoxa10 (FIG. 15D, FIGS. 22E-22F). Similar alterations were observed in Npm1^{c-Frt} Flt3^{Frt-ITD} GMPs, with evidence of sustained, synergistic activation of STAT5 and MAPK-signaling (FIGS. 22G-22H).

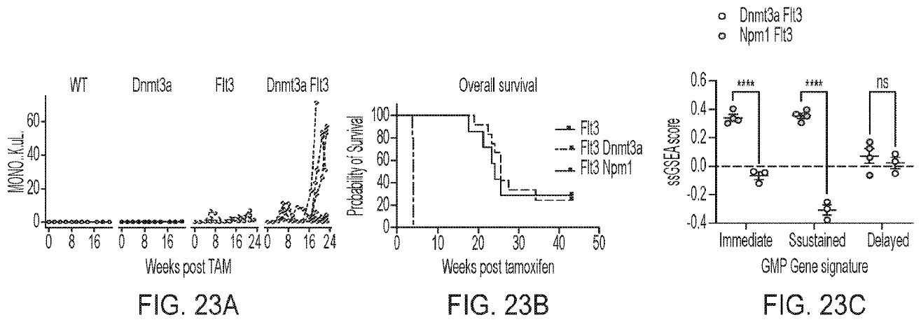

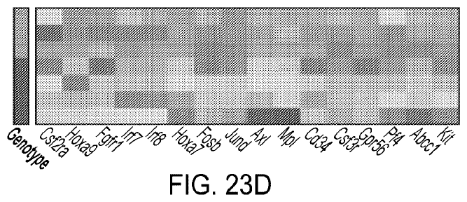

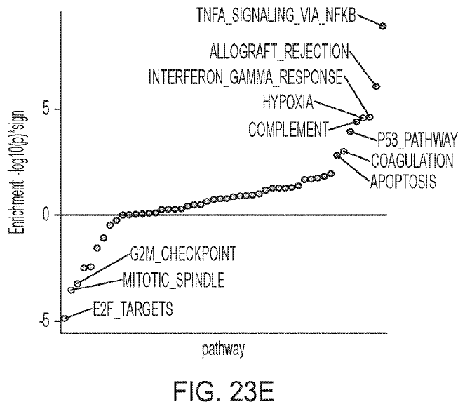

Mutant Flt3 was evaluated in a sequential mutagenesis model with a common CH mutation Dnmt3a^{R878H-Lox}. Cre mRNA was administered to activate the underlying Dnmt3a^{R878H-Lox} allele, and TAM was used to activate Flt3^{Frt-ITD} four weeks after engraftment (FIG. 15E). In these instances, an increase and subsequent decrease in WBC was observed as in the Flt3^{Frt-ITD} alone model, however by 20 weeks post TAM a robust and consistent increase in WBC accompanied by a reemergence of cKIT+ cells in the blood was observed (FIGS. 23A-23B). While the Npm1^{c-Frt} Flt3^{Frt-ITD} mice rapidly succumbed to disease, there were no significant differences in survival between Dnmt3a^{R878H-Lox}-Flt3^{Frt-ITD} and Flt3^{Frt-ITD} only mice (FIG. 23C). Critically, despite the similar disease kinetics as Flt3^{Frt-ITD} cells, Dnmt3a^{R878H-Lox}-Flt3^{Frt-ITD} cells were capable of engrafting into secondary recipients and reestablishing disease albeit at a longer latency than the aggressive Flt3^{Frt-ITD} Npm1^{c-Frt} model (FIG. 15F).

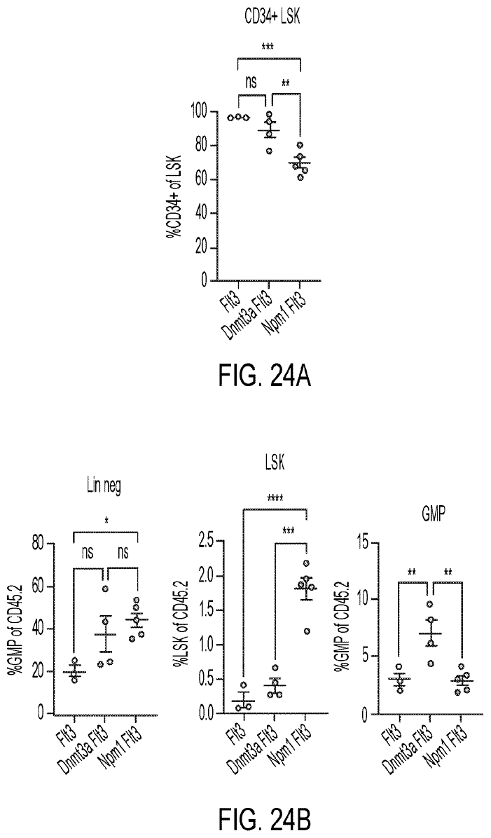

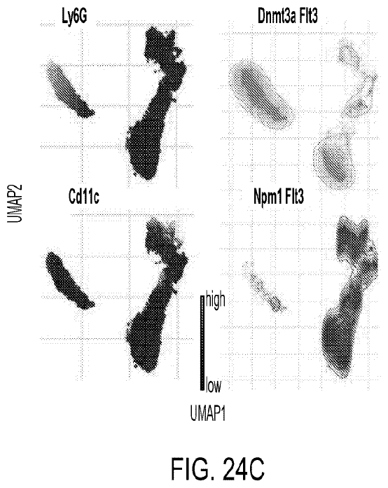

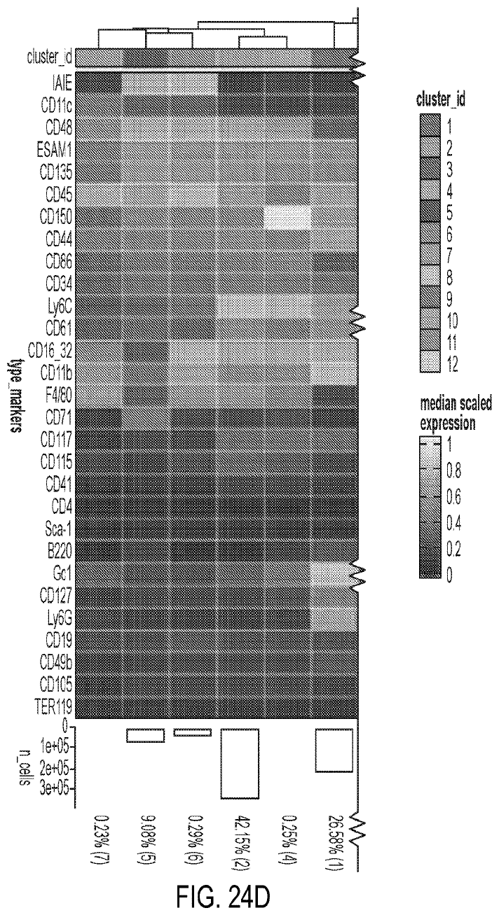

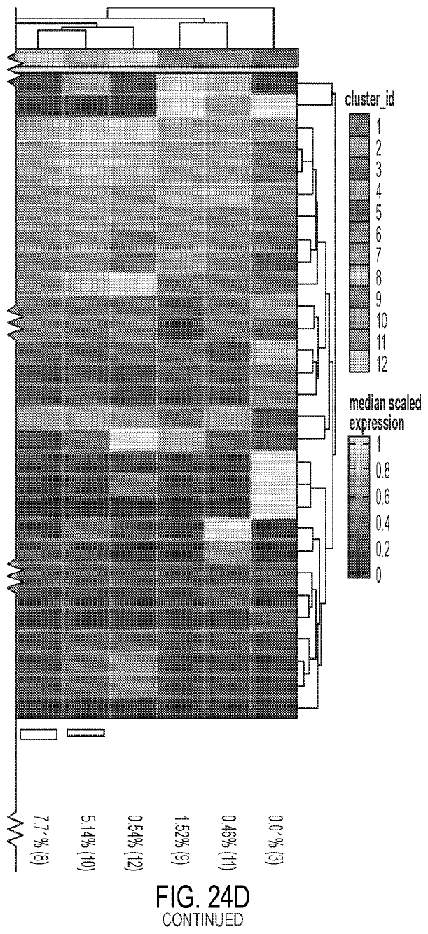

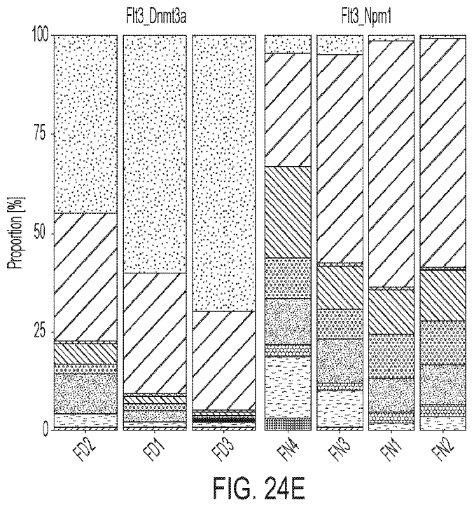

Gene expression analysis revealed that Npm1^{c-Frt} Flt3^{Frt-ITD} LSKs were enriched for genes which were acutely activated with Flt3^{Frt-ITD} alone, reinforcing the idea that Npm1^{c} might lock Flt3-mutant cells into an early activation state (FIG. 15G, FIG. 23D). Meanwhile, Dnmt3a^{R878H-Lox}-Flt3^{Frt-ITD} mutant leukemias appeared enriched for cell cycle signatures, and expression of more immature markers including Gpr56, Cd34, Abcc1, Kit and Mpl (FIGS. 23D-23E). Npm1^{c-Frt} Flt3^{Frt-ITD} leukemias in contrast expressed lower levels of Cd34 (FIG. 24A), matching the clinical immunophenotype, and were enriched for Hox gene expression particularly Hoxa7 and Hoxa9 (FIGS. 23D-23E). Immunophenotypically, an expansion of the progenitor compartment (GMP) for Dnmt3a^{R878H-Lox}-Flt3^{Frt-ITD} cells was observed, while Npm1^{c-Frt} Flt3^{Frt-ITD} leukemias were enriched for LSKs (FIG. 24B). CyTOF revealed that difference persisted into more mature cells with Npm1^{c-Frt} Flt3^{Frt-ITD} leukemias strongly enriched for dendritic like characteristics (Cd11c), while Dnmt3a^{R878H-Lox}-Flt3^{Frt-ITD} leukemia displayed a granulocytic bias (FIG. 15H, FIGS. 24C-24E). These differentiation biases were present even at the level of stem cells, with LSKs from Npm1^{c-Frt} Flt3^{Frt-ITD} mice expressing higher levels of the GM-CSF receptor Csf2ra, while LSKs from FD mice expressed the G-CSF receptor Csf3r (FIG. 23D). Collectively these models demonstrate that different co-occurring mutations are capable of transforming Flt3 mutant cells, albeit with distinct latencies, phases of cooperativity, and immunophenotypic outputs.

### Example 8: Expanding the Sequential Mutagenesis Toolkit with Orthogonal Recombinases

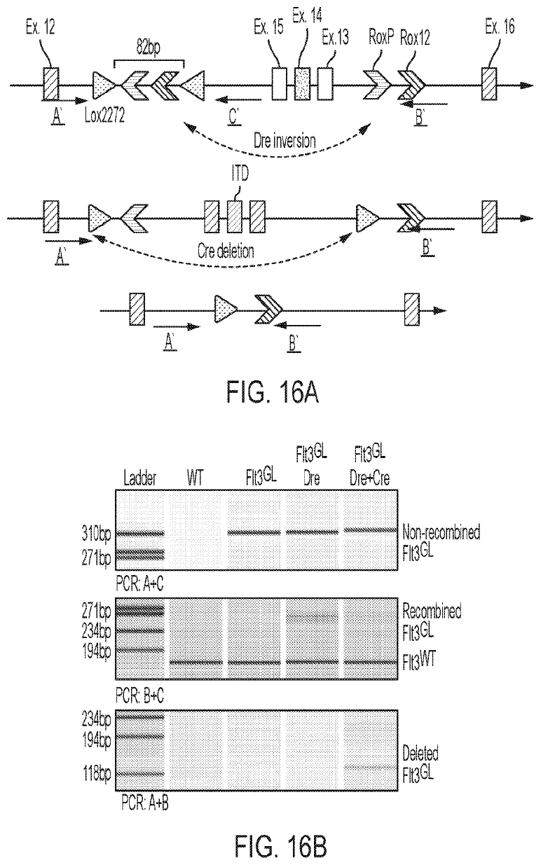

To extend these models of sequential mutagenesis, a third recombinase, Dre, a Cre-homologue capable of recombining Rox sites was used. Additionally, dual recombinase alleles capable of activating and inactivating oncogenes at their endogenous loci were generated. A strategy termed GOLDI-Lox for governing oncogenic loci by dre inversion and lox deletion (GOLDI-Lox) was developed (FIG. 16A). In this model, a pair of Lox2272 sites flank a staggered, heterotypic Rox12—RoxP pair, which themselves flank inverted mutant exon encoding the same ITD mutation as in the Flt3^{Frt-ITD} allele. In the non-recombined state, the close proximity (82 bp) of the Lox2272 sites prevents Cre recombination. Upon Dre activation, the ITD encoding exons are inverted and the Lox2272 sites are inverted and sufficiently separated to permit their recombination. Cre activation results in deletion of the mutant exons and nonsense mediated decay of the residual mRNA. The spacing of the Lox2272 sites in the non-recombined state allows this Dre-on/Cre-off allele to be combined with Cre-inducible alleles for sequential mutagenesis. A similar strategy was used to target Jak2^{V617F} as described above. To evaluate the efficacy of this strategy, this mouse (Flt3^{GL-ITD}) was crossed to a Dre Reporter (RLTG; TdTomato reporter), and a TAM-inducible Ubc:CreER allele. HSPCs from these mice were infected with a Dre-IRES-GFP encoding retrovirus for 48 hours with or without Cre induction by 4-OHT. PCR demonstrated that Dre activation was indeed capable of inverting the target locus, while administration of 4-OHT resulted in deletion of the target locus (FIG. 16B).

To benchmark the Flt3^{GL-ITD} allele, it was crossed to the Ubc:CreER allele and a panel of Cre responsive alleles including Npm1^{C-Lox}, Idh2^{R140Q-Lox} and Tet2^{flox/flox} mice. The cooperating allele was (in)activated with TAM; HSPCs were then isolated for Dre nRNA electroporation ex vivo (FIG. 24A). In this setting, it was found that much like the Flt3^{Frt-ITD} allele, Flt3^{GL-ITD} mutant cells showed an increase in WBC/Monocytes, a process that was maintained in both the Idh2^{R140Q-Lox}-Flt3^{GL-ITD} and Tet2^{flox/flox}-Flt3^{GL-ITD} models (FIG. 24B). Meanwhile Npm1/Flt3 cells underwent exponential expansion as seen when the alleles were simultaneously activated, resulting in a fully penetrant, lethal AML. Previous studies with a constitutive Flt3^{ITD} have demonstrated robust disease penetrance for both Idh2^{R140Q-Lox} and Tet2^{flox/flox} mice, however by 6 months post transplant, only a subset of Idh2^{R140Q-Lox}-Flt3^{GL-ITD} (2/10) and Tet2^{flox/flox}-Flt3^{GL-ITD} (1/6) mice developed a transplantable leukemia (FIG. 24B). Collectively these results demonstrate a synergy between Npm1^{C} and Flt3^{ITD} uniquely attributed to the acute phase of Flt3^{ITD} activation. While the variable penetrance of the Tet2 and Idh2 models suggests either differential requirements for initiating allele burden, or sensitivity to the transplant-based experimental schema.

### Example 9: Sequential Mutagenesis Reveals Stage and Mutant-Specific Alterations in Differentiation

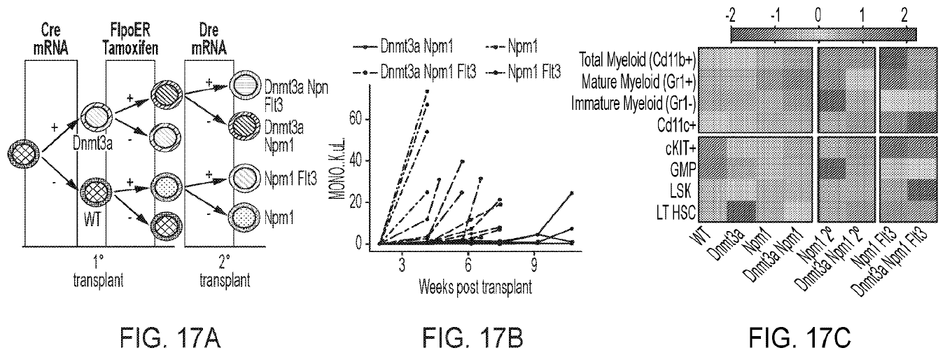

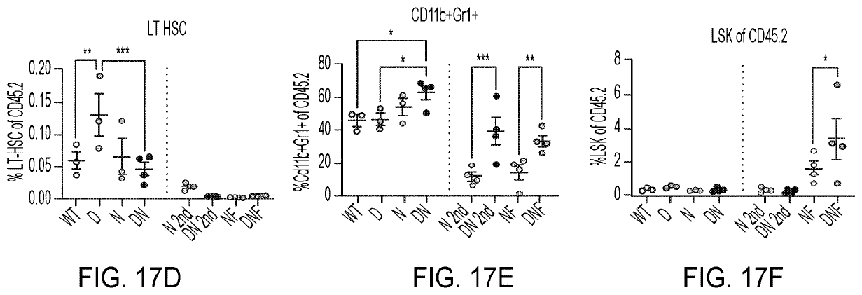

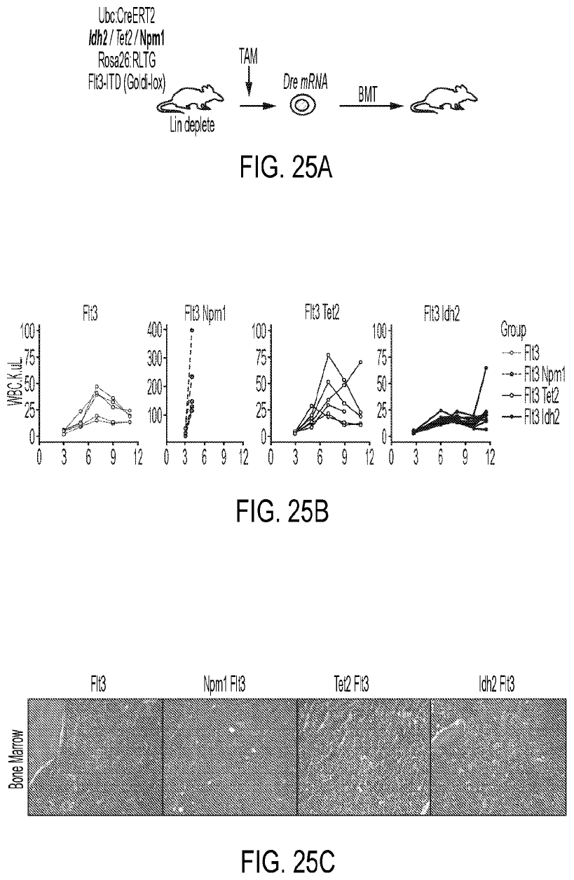

While Npm1^{C}-Flt3^{ITD} combinations potently induce AML in mice, most patients present with an antecedent mutation, the most common of which is found in DNMT3A, generating a triad of mutations which confers dismal prognosis for patients. This combination of alleles was modeled using 3 separably inducible alleles Dnmt3a^{R878H-Lox}, Npm1^{c-Frt}, and Flt3^{GL-ITD} (FIG. 17A). Initiating mutations were activated in Dnmt3a^{R878H-Lox} with Cre mRNA electroporation into HSPCs at transplant. Npm1^{c-Frt} mutations were then activated in a subset of mice with the TAM-inducible FlpoERT2 allele. By 16 weeks post Npm1^{c-Frt} activation, Dnmt3a^{R878H-Lox}-Npm1^{c-Frt} mutant mice developed mild alterations in RBC and WBC, with a robust myeloid bias observed in double mutant mice, specifically enriched for Gr1-high granulocyte like cells (FIG. 25A).

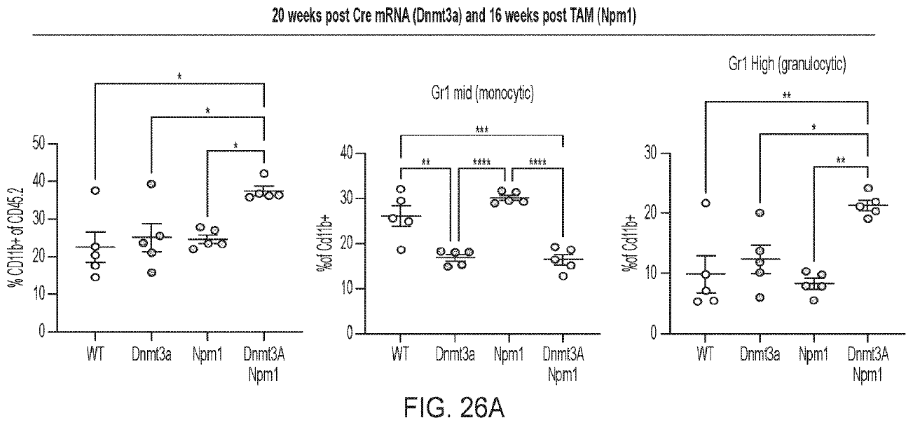

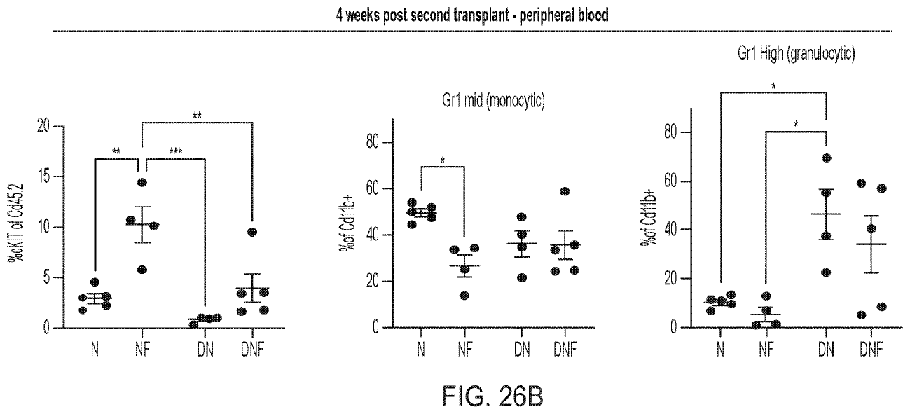

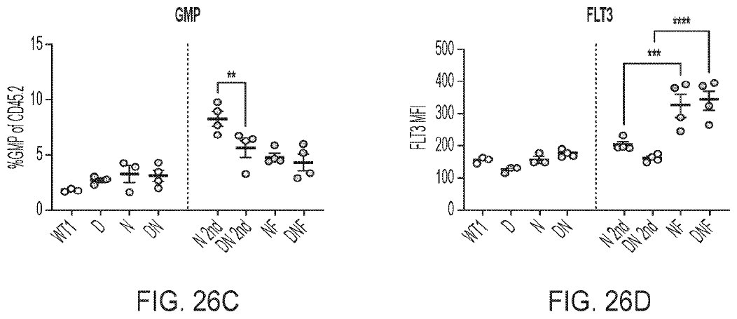

To model a Flt3 mutation as a late hit in leukemogenesis, Dre mRNA was electroporated into HSPCs at transplant to generate 4 new experimental groups (FIG. 17A). Here, as in previous models Npm1^{c-Frt}-Flt3^{GL-ITD} mice developed AML within 4 weeks post transplantation (FIG. 17B). Triple mutant Dnmt3a^{R878H-Lox}-Npm1^{c-Frt}-Flt3^{GL-ITD} mice displayed a slight lag in disease development however displayed a similar exponential growth with an increase in cKIT+ cells in the blood (FIG. 25B). Meanwhile single mutant Npm1^{c-Frt} and double mutant Dnmt3a^{R878H-Lox}-Npm1^{c-Frt} cells reliably engrafted, but possessed an even greater latency with Npm1^{c-Frt} mutant cells eventually emerging disease before their double mutant counterparts (FIG. 17B). While serial transplantation of Npm1^{c-Frt}-only mice results in expansion of GMPs, this skew was blunted by an antecedent Dnmt3a^{R878H-Lox} allele (FIG. 17C, FIG. 25C). In contrast, Flt3^{GL-ITD} mutations expressed higher levels of Flt3, and exhibited an expansion Cd11c+ dendritic-like and total cKIT+ cells independent of cooperating mutations (FIG. 17C, FIG. 26D). In addition to driving LT-HSC expansion as a mono-allele, the granulocytic bias evident in the Dnmt3a^{R878H-Lox} Flt3^{Frt-ITD} persisted here in both Dnmt3a^{R878H-Lox}-Npm1^{c-Frt} double mutant mice, as well as in Dnmt3a^{R878H-Lox} Npm1^{c-Frt} Flt3^{GL-ITD} triple mutant mice (FIGS. 17D-17E). Finally, while Flt3^{GL-ITD} appeared to be the dominant driver of LSK expansion, antecedent Dnmt3a^{R878H-Lox} mutations resulted in a further increase in this population. These findings demonstrate that while early mutations such as DNMT3A are not strictly necessary for disease development, they do influence the rate of disease development and differentiation landscape of subsequent leukemias.

### Example 10: Inducible Recombinases Allow for Facile Disease Development

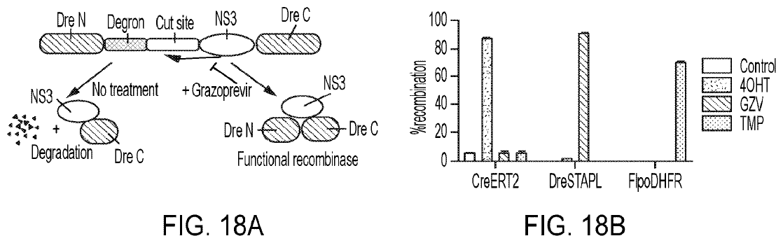

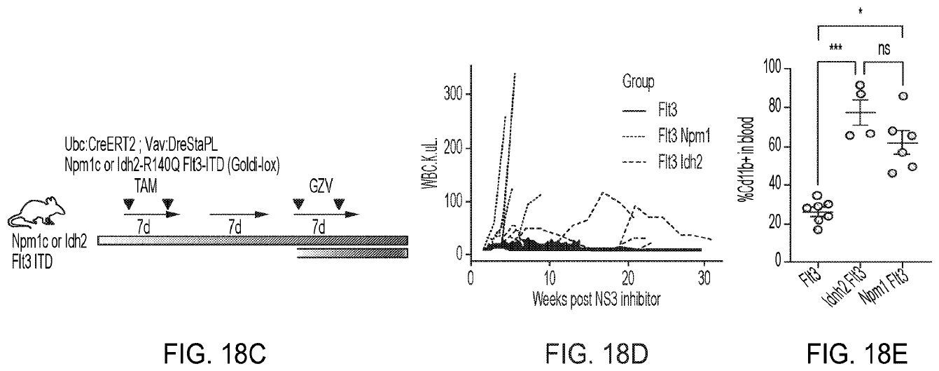

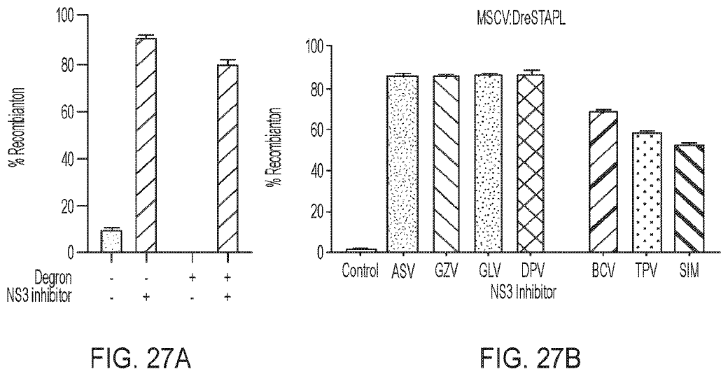

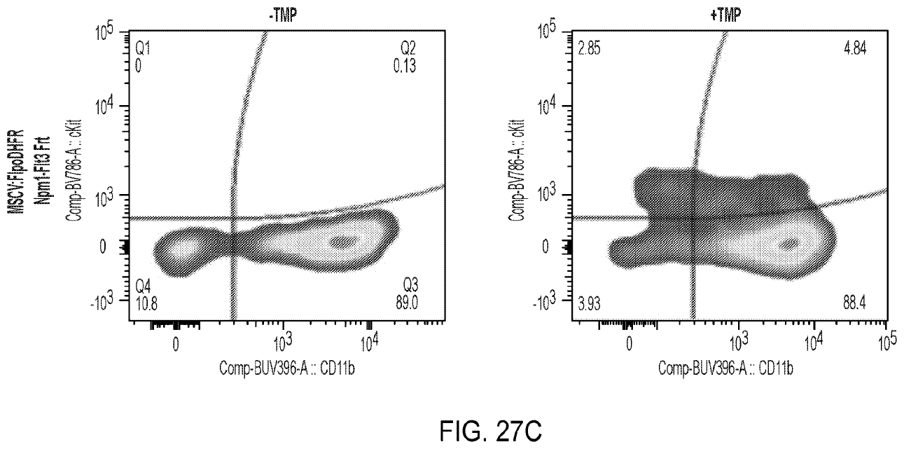

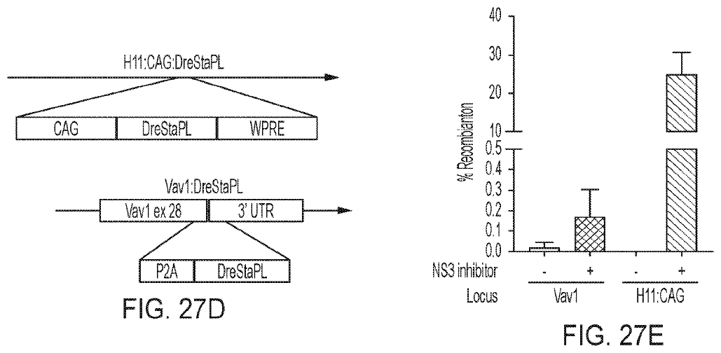

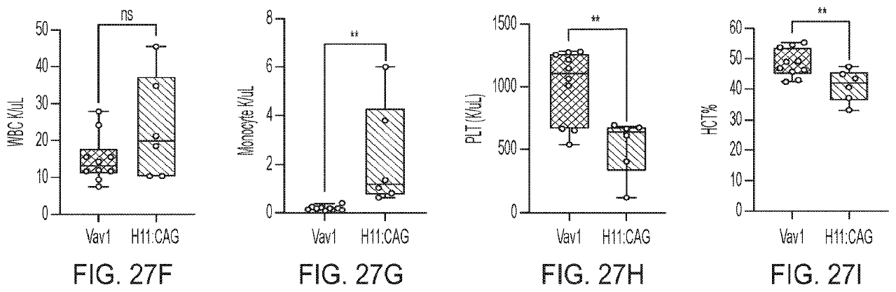

To enable sequential mutagenesis without relying upon transduction at transplant, orthogonal chemical tools were implemented to activate the recombinases orthogonal to existing tamoxifen inducible ER fusion proteins employed above. Previous studies have implemented RU486 inducible PR fusions for Dre, however these studies have been limited by in vivo inducibility. FKBP12-Dre fusions displayed excessive background recombination in the presence of the stabilizing ligand Shield1. A Stabilized Peptide Linkage (StaPL) approach was implemented by splitting Dre with an HCV-derived NS3 protease leading to constitutive autoproteolysis of Dre (FIG. 18A). Inhibition of the proteolytic activity with an asunaprevir (ASV) or grazoprevir (GZV), resulted in efficient recombination (FIG. 27B). To reduce baseline recombination, an ornithine-decarboxylase protected degron was cloned adjacent to the NS3 cut site, where after NS3 cleavage the N terminal fragment of Dre is degraded (DreSTAPL-ODC). This eliminated leakiness and allowed for ligand inducible Dre activation in vitro and in vivo with near complete loss of background recombination (FIG. 27A). Critically, the DreStaPL-ODC was orthogonally inducible to both tamoxifen-inducible CreER and the trimethoprim-stabilizing Flpo dihydrofolate reductase (FlpoDHFR) fusions (FIG. 18B). Expectedly, when this FlpoDHFR fusion was delivered as a retrovirus in a transplant model, TMP treatment was capable of inducing aggressive AML in the Flp Flt3/Npm1 model (FIG. 27C).

To complement existing Flpo and Cre tools, two new DreStaPL-ODC mouse lines were generated, targeting the fusion recombinase to either the endogenous Vav1 locus, or to the Hip11 safe harbor under the control of a synthetic CAG promoter (FIG. 27D). While the CAG:DreStaPL-ODC mice displayed robust recombinase activation both in vivo (FIG. 27E), mice possessed abnormal physiology with alopecia at birth and eventual ocular lesion, potentially due to excessive levels of the NS3 protease. Vav1:DreStaPL-ODC mice displayed no abnormal phenotype, however possessed reduced recombination activity compared to CAG:DreStaPL-ODC (FIG. 27E). When crossed to Flt3^{GL-ITD} mice, the discrepant recombination efficiency of the two models resulted in expectedly distinct outcomes on peripheral blood counts. Vav1:DreStaPL-ODC mice showed no significant differences with GZV treatment, however CAG:DreStaPL-ODC mice displayed characteristic patterns of FLT3 activation 6-8 weeks post treatment including monocytosis, mild anemia and mild thrombocytopenia (FIGS. 27F-27I).

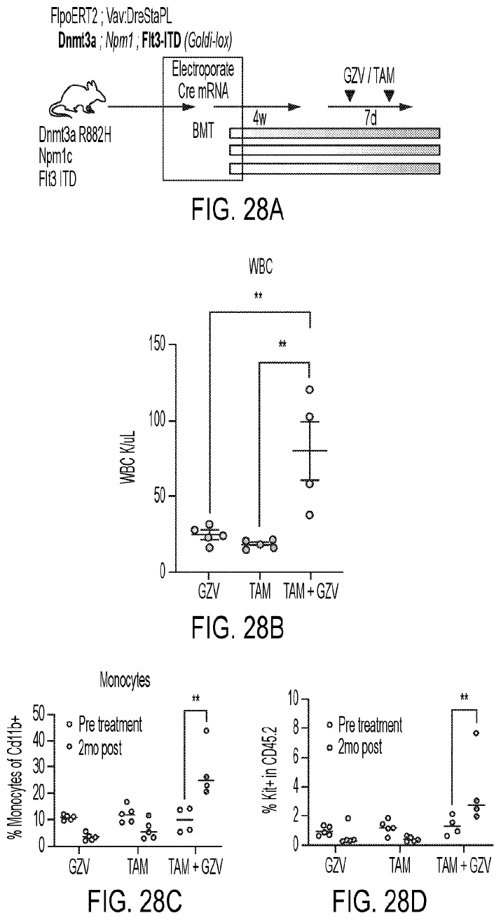

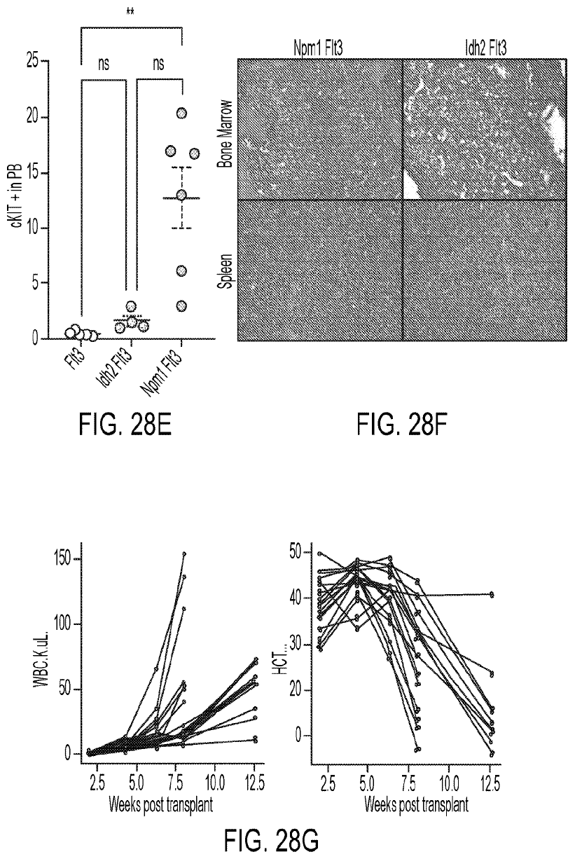

Given the ability of limiting dilutions of TAM to generate an AML in the Npm1^{c-Frt} Flt3^{Frt-ITD} model (FIG. 22D), it was sought to test if the limited inducibility of Vav1:DreStaPL-ODC would be sufficient to initiate subclonal disease. The Vav1:DreStaPL-ODC was crossed to the triple mutant model (Dnmt3a^{R878H-Lox}, Npm1^{c-Frt}, and Flt3^{GL-ITD}) As in FIG. 16A, Dnmt3a^{R878H-Lox} was activated by Cre mRNA at transplant, however now Npm1^{c-Frt} or Flt3^{GL-ITD} were activated by the administration of either TAM for FlpoERT2 or GZV for DreStaPL-ODC respectively (FIG. 28A). It was found that co-administration of TAM and GZV resulted in a significant increase in WBC, myeloid skewing, and expansion of cKIT+ cells in the blood, consistent with an aggressive leukemia in only 4 weeks post treatment (FIGS. 28B-28D). Meanwhile at this same time point, administration of either ligand alone did not significantly alter blood compartment parameters, further demonstrating the potency of Npm1^{c} and Flt3^{ITD} in driving disease development.

Finally, it was sought to eliminate any reliance upon transplant for the induction of disease. Four allele mice were generated harboring Ubc:CreER, Vav1:DreStaPL-ODC, Flt3^{GL-ITD}) and either Cre-inducible Npm1^{c-Lox} or Idh2^{R140Q-lox} mutations. Mice were administered tamoxifen to activate either Npm1^{c-Lox} or Idh2^{R140Q-lox}, and then 2 weeks later treated with GZV to induce Flt3^{GL-ITD} (FIG. 18C). Again, Flt3^{GL-ITD} only mice did not develop significant alterations in hematopoietic parameters (FIG. 18D), suggesting the allele burden generated by the Vav1:DreStaPL-ODC was insufficient to recapitulate the dynamics seen in the high allele burden FlpoER model (FIG. 14A) or the Dre mRNA model (FIG. 23B). Despite this decreased potency, Npn1^{c-Lox}-Flt3^{GL-ITD} mice developed an aggressive AML at <6 weeks post GZV induction, highlighting the potency of these cooperating alleles (FIG. 18D). In contrast, Idh2^{R140Q-lox}-Flt3^{ITD} mice, developed a longer latency AML with a mild leukocytosis and reduced peripheral blood cKIT+ cells compared to the Npm1^{c-Lox}-Flt3^{GL-ITD} model (FIG. 181), FIG. 28E). Despite the discrepancy in disease latency, both models generated a significant myeloid skewing, with cells capable of engrafting disease into secondary recipients (FIG. 18E, FIGS. 28F-28G).

### Example 11: Evaluating Oncogene Dependency of Mutant Flt3

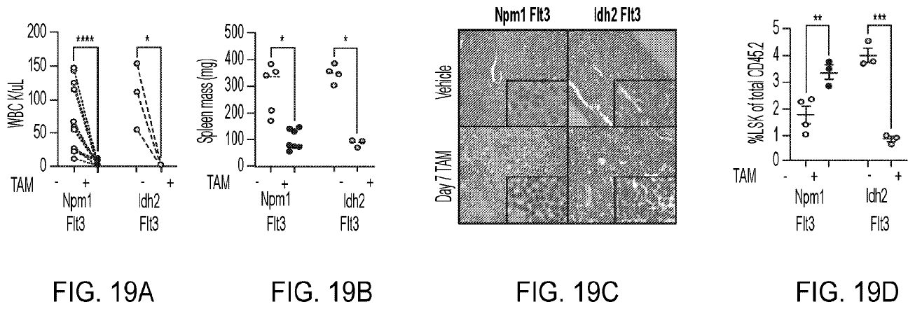

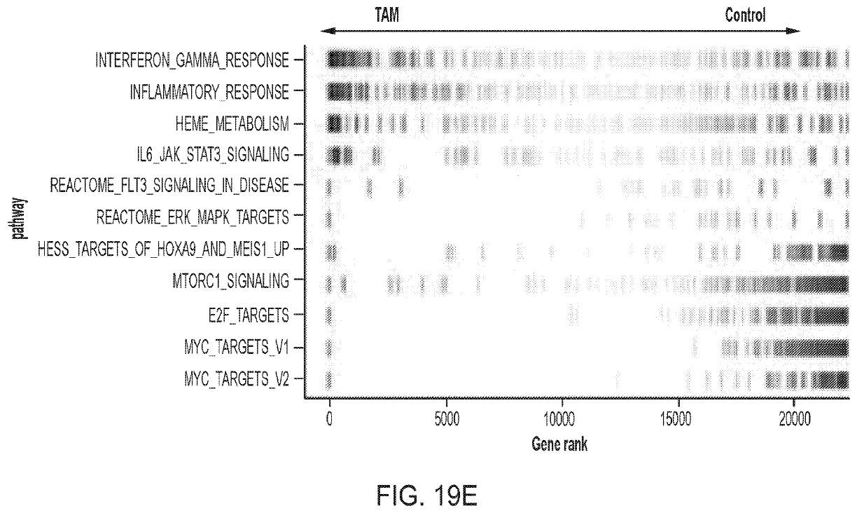

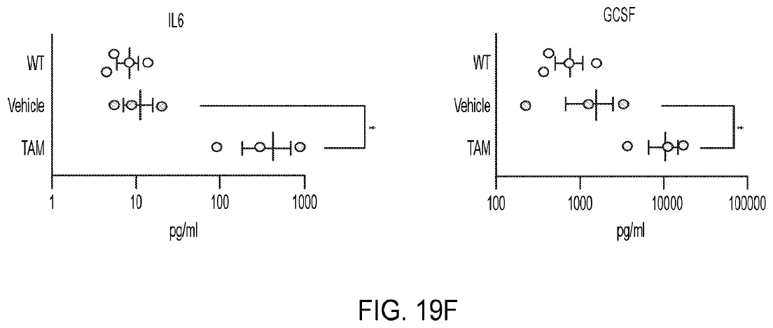

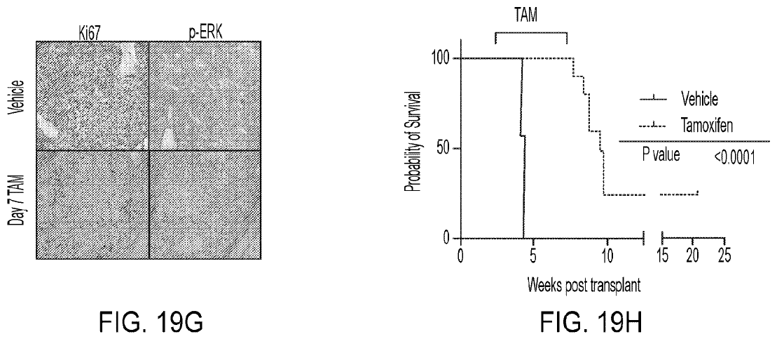

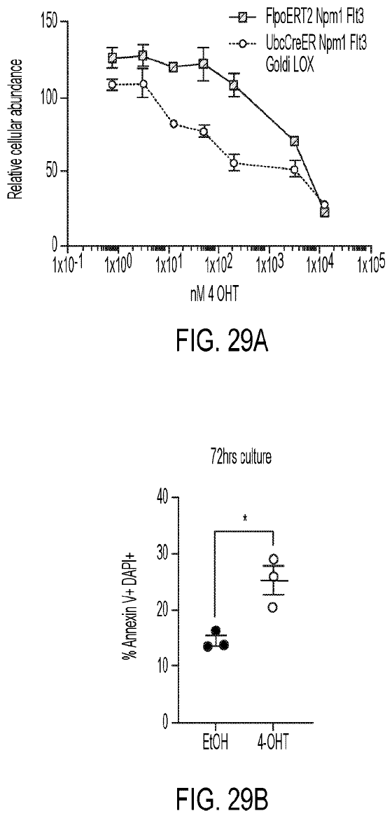

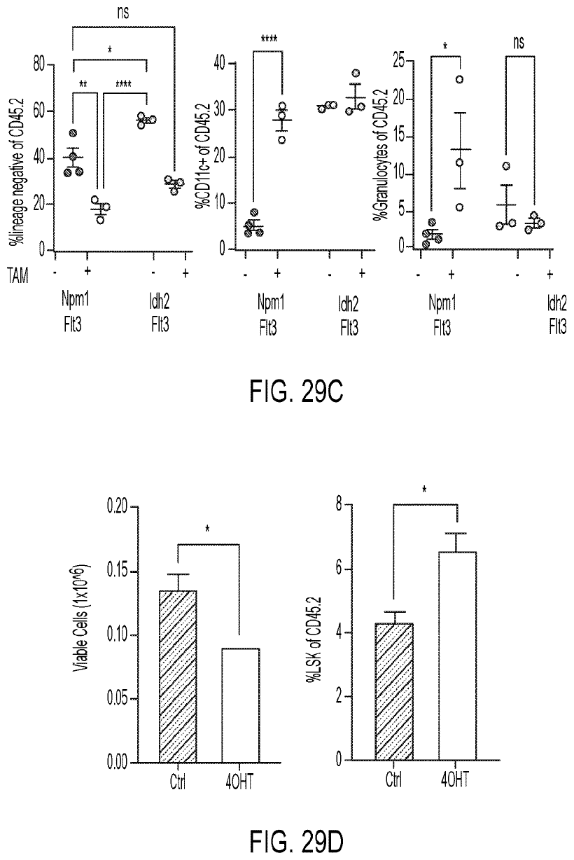

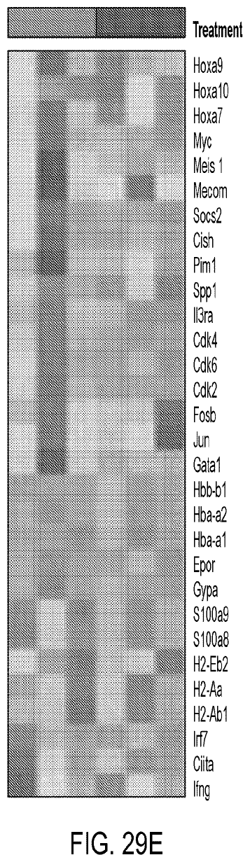

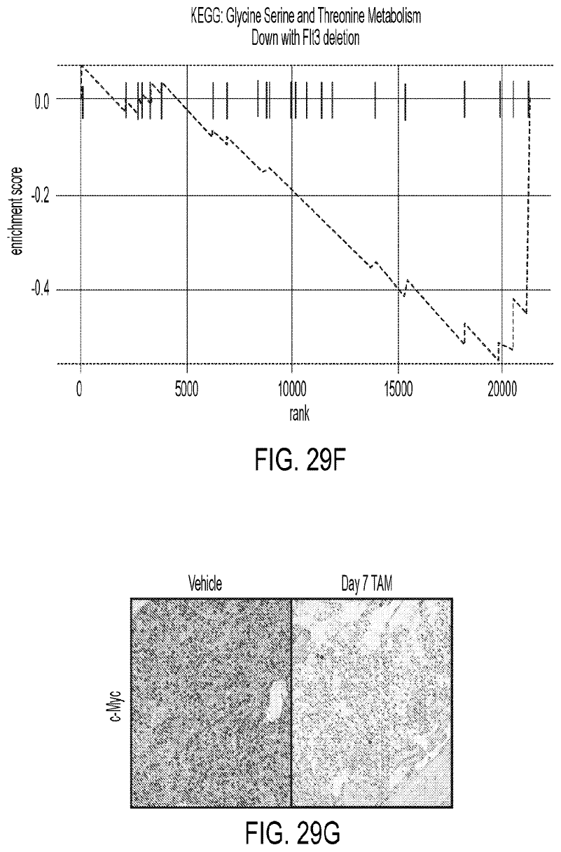

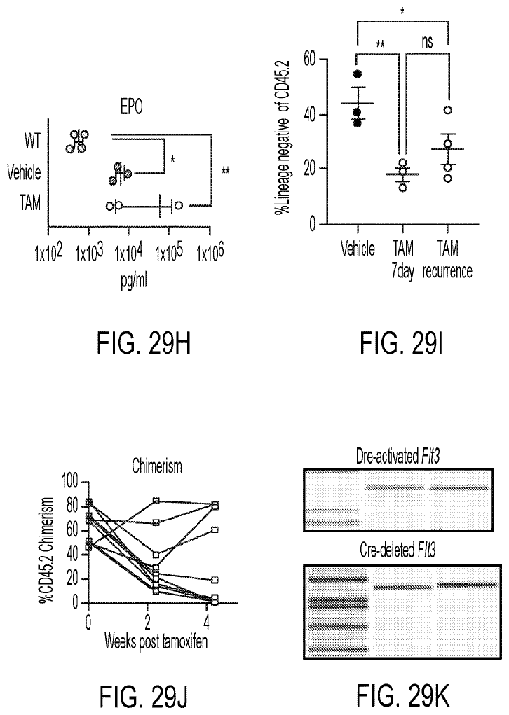

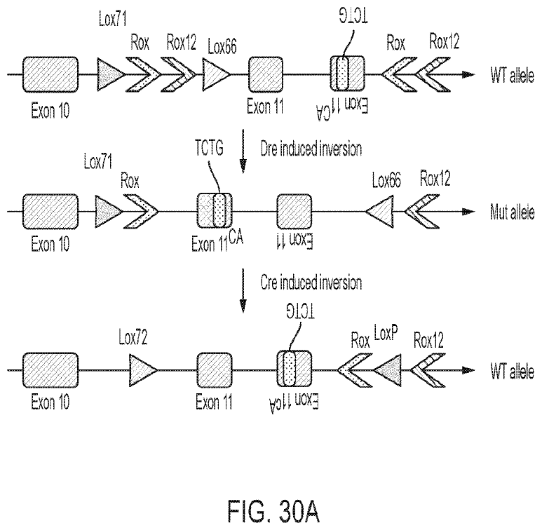

It was next sought to perturb disease evolution through the genetic deletion of mutant Flt3. Leukemias from the models in FIG. 18C were transplanted into lethally irradiated recipients and mice were monitored for disease development at which point they were treated with TAM to delete the Flt3^{GL-ITD} allele. Flt3 ablation was associated with a near complete reduction of WBC and decrease in spleen mass by 7 days in both the Npm1^{c-Lox}-Flt3^{GL-ITD} and Idh2^{R140Q-lox}-Flt3^{GL-ITD} mice (FIGS. 19A-19B). Similarly, in vitro, 4-OHT treatment led to a reduction in cell number in Ubc:CreERT2 Npm1^{c-Lox}-Flt3^{GL-ITD} cells but not FlpoERT2 Npm1^{c-Frt} Flt3^{Frt-ITD} cells (FIG. 29A). This decrease in cellularity was due, at least in part, to apoptosis with an evident increase in AnnexinV+DAPI+staining in vitro (FIG. 29B).

Pathological analysis of the bone marrow revealed a decrease in cellularity, and marked differentiation with nuclear condensation (FIG. 19C). Both models showed a reduction in lineage negative HSPCs, with Npm1^{c-Lox}-Flt3^{GL-ITD} expanding both granulocytes and a Cd11c+ dendritic-like population upon TAM-treatment (FIG. 29C). While the Idh2^{R140Q-lox}-Flt3^{GL-ITD} mice displayed a reduction in LSKs, Npn1^{c-Lox}-Flt3^{GL-ITD} mice displayed an increase in immunophenotypic LSKs with a near complete absence of Kit+Sca1− progenitor cells (FIG. 19D), with similar results observed with 4-OHT administration in ex vivo culture (FIG. 29D).

This increase in phenotypic LSKs suggests either a maintenance of a stem-like compartment or a dramatic increase in inflammation and upregulation of Seal. Gene expression profiling on HSPCs from the Npm1^{c-Lox}-Flt3^{GL-ITD} model revealed TAM treatment was associated with a robust inflammatory gene signature driven by an interferon gamma response and STAT3/IL6 signaling (FIG. 19E). Serum cytokine levels showed an increase in IL6, while no significant alterations were found for TNFalpha and IFNg (FIG. 19F). Meanwhile. a decrease in HOXA9 and MEIS1 target genes was observed, underscored by decreased expression of the transcription factors themselves (FIG. 19F, FIG. 29E). Additionally, a reduction in gene expression signatures was observed for E2F targets, FLT3 signaling, serine metabolism, mTORC1 signaling, MYC targets and ERK pathway engagement (FIG. 19F, FIG. 29E-29F). Histochemical analysis confirmed a decrease in both cell cycle (Ki67) and pERK signaling, as well as downregulation of cMYC (FIG. 19G, FIG. 29G). Finally, HSPCs showed re-engagement with erythropoiesis by an increase in genes associated with Heme synthesis as well as increased expression of Gata1 and Epor independent of serum EPO levels (FIG. 19E. FIG. 2911). Collectively, these alterations indicate that mutant-Flt3 deletion is associated with a reduction in proliferation self renewal related Hox gene expression, and a shutdown of critical signaling pathways underlying aggressive disease progression.

Long term treatment with TAM resulted in an increase in overall survival, with a subset of Npm1^{c-Lox}-Flt3^{GL-ITD} (2/10) near eradication of leukemic cells (FIG. 19H). Ultimately, most mice recurred with disease, albeit with a transient reduction in peripheral blood chimerism reduction and eventual decrease in lineage negative HSPCs (FIG. 19F, FIG. 29I-29J). At recurrence, PCR evidence of both Cre-mediated Flt3^{GL-ITD} deletion, as well as residual Dre-activated Flt3^{GL-ITD} that was not deleted by Cre was observed (FIG. 29K). These findings suggest that despite constant suppression with TAM-activated Cre, Flt3-mutant clones were capable of escape, potentially driving recurrence. The persistence PCR bands indicating Cre-deleted Flt3 suggests that 1) not all cells undergo apoptosis upon Flt3-deletion and 2) raise the possibility of Flt3-WT clones contributing to disease emergence.

Advances in single cell genomics have offered refined resolution into the evolutionary processes underlying cancer development and response to therapy. While GEMMs have served as a robust means to model disease, spatiotemporal control of stepwise somatic alterations remains elusive. Here, chemically orthogonal, multi-recombinase models of somatic event control which complement existing alleles in hematopoietic malignancy were employed. These studies identified new insights in acute and chronic phase activation of mutant Flt3, the most commonly mutated gene in acute myeloid leukemia. It was found that while Flt3 was insufficient to cause leukemia, cooperating mutations licensed Flt3-iTD for transformation, with different mutational combinations altering disease kinetics, transcriptional cooperativity, and immunophenotypic output.

While clonal hematopoiesis is a common somatic event in an aged population and is assessed with an increased probability for myeloid malignancy, transformation to AML remains a rare event. The models described here build off of previous research to understand this transition from a premalignant state towards leukemic development. While previous studies have enabled progressive mutation acquisition with CRISPR/Cas9 or viral overexpression, none have permitted stepwise sequential mutagenesis in the setting of native hematopoiesis. Given the selective pressure introduced with irradiation-based conditioning for transplantation, consolidation of somatic mutation systems in the absence of transplantation will be a critical tool to understand the mechanism underlying clonal dominance and its eventual emergence to leukemic transformation.

While doxycycline inducible systems have been used to assess oncogene-dependency, endogenous expression levels remain a critical concern, particularly for mutant-oncogenes with restricted cell type expression patterns such as Flt3. Other gene-trap approaches have retained locus specific expression levels but have thus far been largely deployed for loss of function alleles. Here, Cre and Dre recombinase were paired in the GOLDI-Lox framework to allow for both inducible antecedent events followed inducible activation and reversion of the Flt3 oncogene. This setup allowed for the performance of three functions with two recombinases, decreasing the genetic burden necessary to establish the models. With this approach, it was demonstrated that genetic inactivation of mutant-Flt3 results in immediate regression of leukemia albeit with distinct immunophenotypic consequences depending upon the cooperating mutations. Despite this robust early response, the majority of mice eventually succumbed to disease escaping genetic deletion. This can be explained, in part, through insufficient Cre activity with evidence of residual mutant Flt3. Even with robust FLT3 deletion, there was insufficient residual hematopoiesis from either remaining WT cells or Npm1′ only mutant cells to recover from leukemic insult. These findings mirror the clinical scenario where the FLT3 tyrosine kinase inhibitor gilteritinib robustly generated complete remissions, yet increases in overall survival are only significantly established in patients who underwent hematopoietic stem cell transplant and continued gilteritinib therapy.

### Example 12: Materials and Methods for Examples 6-11

### Mouse Model Generation.

The Flt3^{Frt-ITD} and Flt3^{GL-ITD} mice were generated at Ingenious targeting labs. The targeting construct for Flt3^{Frt-ITD} was generated A 9.8 kb genomic DNA used to construct the targeting vector was first subcloned from a positively identified C57BL/6 BAC clone (RP23-280N16). The region was designed such that the long homology arm (LA) extends ˜7 kb 5′ to the 5′ FRT cassette, and the short homology arm (SA) extends about 2 kb 3′ to the insertion of the inversion cassette. The inversion cassette is flanked by mutant F3 sites that point away from each other. The 3′ FRT site is placed right before the 3′ F3 site. The inversion cassette consists of exon 13-mutant exon 14* (humanized Flt3-IDT)-exon 15 and the flanking genomic sequences from upstream of exon 13 to downstream of exon 15 for correct splicing (Inv.saE13-14*-E15Sd). This cassette was inserted in the reverse direction downstream of exon 15. The Lox2272-flanked Neo cassette was inserted immediately upstream of the inversion cassette and is 235 bp away from wt exon 15. The targeting region is 985 bp containing exons 13-15. Ten micrograms of the targeting vector was linearized and then transfected by electroporation of C57Bl/6 (B6) embryonic stem cells. After selection with G418 antibiotic, surviving clones were expanded for PCR analysis to identify recombinant ES clones. After successful clone identification, the neomycin cassette was removed with a transient pulse of Cre recombinase and clones were reconfirmed following expansion. Finally, ES cells were injected in C57B6 mice via tetraploid complementation (NYU).

### Targeting Construct.

The targeting construct for Flt3^{GL-ITD}, was generated as follows: A 9.8 kb genomic DNA used to construct the targeting vector was first subcloned from a positively identified C57BL/6 BAC clone (RP23-280N16). The region was designed such that the long homology arm (LA) extends ˜5.7 kb 5′ to the cluster of Lox2272-Rox-Rox12-Lox2272 sites, and the short homology arm (SA) extends about 1.9 kb 3′ to the Neo cassette and 3′ Rox12 site. The inversion cassette is in between the second set of Lox2272 and Rox sites, and it consists of exons 12-15 with the IDT mutation engineered in exon 14 (Flt3-IDT) and the flanking genomic sequences from upstream of exon 12 to downstream of exon 15 for correct splicing (SaE12-W51-15Sd). The inversion cassette replaces wt exons 12-15 and the same flanking genomic sequences included in the cassette. The deleted region is 2.19 kb. The FRT-flanked Neo cassette is inserted immediately downstream of the inversion cassette. Each pair of the recombination sites are in opposite direction. Ten micrograms of the targeting vector was linearized and then transfected by electroporation of FLP C57Bl/6 (BF1) embryonic stem cells. After selection with G418 antibiotic, surviving clones were expanded for PCR analysis to identify recombinant ES clones. The Neo cassette in targeting vector has been removed during ES clone expansion. Targeted iTL BF1 (C57BL/6 FLP) embryonic stem cells were microinjected into Balb/c blastocysts. Resulting chimeras with a high percentage black coat color were mated to C57BL/6 WT mice to generate Germline Neo Deleted mice. Tail DNA was analyzed as described below from pups with black coat color.

### Cloning.

Codon optimized versions of Cre, CreERT2 and Dre were synthesized (Thermo) and subcloned into pENTR using the directional TOPO cloning kit. Flpo was PCR amplified from a FlpoERT2 construct provided by Dr. Alex Joyner at MSKCC, and then TOPO cloned into pENTR using the same approach. DreStaPL and DreStaPL-ODC were generated by a gibson reaction with the backbone generated through inverse PCR of the pENTR vector, and the StaPL containing insert synthesized as a gBlock for IDT. A similar approach was used for inserting DHFR downstream of Flpo. These pENTR constructs were then cloned into a MSCV:IRES GFP destination vector using a gateway cloning approach (Thermo: LR Clonase II).

### Virus Generation and Viral Infection.

MSCV retroviral vectors were transfected with pCL-Eco packaging vector into 293T-17 cells using JetPRIME following the manufacturers protocol (5 ug each, 1:1). Conditioned media was collected 72 hours later and passed through a 0.45 uM filter. Virus was either used fresh or stored at −80 C for later usage. For viral infection in FIG. 4 and Extended Data 8, lineage negative bone marrow was isolated using the Mouse Hematopoietic Progenitor Cell Isolation Kit (StemCell 19856) and plated over retronectin coated 6 or 12 well plates for 2 hours in RPMI+10% FBS supplemented with Penicillin/Streptomycin, 20 ng SCF, 10 ng IL6 and 10 ng IL3. Virus and 100×HEPES was added to the wells, then plates were spun for 1 hour at 800×G at 37 C. Following spin, small molecule ligands used to activate recombinases were added to a final concentration as indicated ethanol (v/v 0.0015%), DMSO (v/v 0.1%), 4-hydroxytamoxifen (4-OHT; 400 nM), trimethoprim (TMP; 1 uM) or grazoprevir (GZV, 10 uM). Cells were harvested for flow cytometric analysis 48 hours post infection. For viral infection followed by transplantation in Extended Data 8, a similar approach was utilized with the following modifications. Donor mice were treated with 5-FU 6 days prior to harvest. Mice were euthanized as described above, and bone marrow was isolated by flushing bones with 10 ml of FACS buffer with a 26.5 gauge needle. Whole bone marrow was plated retronectin plates as described above, and cells were harvested for transplant 2 hours after infection.

### Tissue Harvest and Bleeds.

Peripheral blood was isolated by submandibular bleeds. Complete blood counts were assessed on a ProCyte by IDEXX. For flow cytometry analysis whole blood was lysed with RBC lysis buffer (Biolegend). For terminal tissue isolation, mice were euthanized with CO2 asphyxiation, tissues were dissected and fixed with 10% Zinc formalin for histopathological analysis. For whole bone marrow was isolation, the femur, hip and tibia were dissected and cleaned. Cells were isolated by centrifugation at 8000×G for 1 minute. Single cell suspension were generated from crushed whole spleen and filtered through a 70 uM filter. RBC lysis was performed (Biolegend) and cells were prepared for downstream processing or frozen in 10% DMSO+90% fetal bovine serum.

### Flow Cytometry and Sorting.

Freshly isolated or cryopreserved cells were washed twice with FACS buffer (phosphate buffered saline (PBS)+2% fetal bovine serum). Cells were incubated with antibody cocktails for 15 minutes at 4° C. Complete antibody details can be found in Supplemental table X. Following antibody incubation, cells were washed with FACS buffer and resuspended in a DAPI containing FACS buffer solution for analysis and sorting. Flow cytometry analysis was performed on a BD LSRFortessa with analysis using FACSDiva and FlowJo (v10.9). Cell isolation with FACS was performed on a Sony SH800.

### Nucleic Acid Isolation and RNA-Sequencing Library Generations.

DNA was isolated using the DNeasy Blood & Tissue Kit (Qiagen). RNA was isolated using the Direct-zol RNA Microprep Kit (Zymo Research, R2061) and quantified using the Agilent High Sensitivity RNA ScreenTape (Agilent 5067-5579) on an Agilent 2200 TapeStation. 3′ multiplexed RNA-sequencing was performed with the Takara SMART-Seq v4 3′ DE Kit (Takara 635040) followed by Nextera XT (Illumina FC-131-1024) library preparation. For full length RNA-sequencing, cDNA was generated from 1 ng of input RNA using the SMART-Seq HT Kit (Takara 634455) at half reaction volume. cDNA and tagmented libraries were quantified using High Sensitivity D5000 ScreenTape (5067-5592) and High Sensitivity D1000 ScreenTape respectively (5067-5584). Libraries were sequenced on a NovaSeq at the Integrated Genomics Operation (IGO) at MSKCC.

### Mass Cytometry and Data Analysis.

Cryopreserved mouse whole bone marrow or spleen cells were thawed and counted. Approximately 1 million from each mouse analyzed were used. Live cells were rested for 2 hours in RPMI+10% FBS (Gibco) (cRPMI) and then stained for viability with 5 M Cell-ID Cisplatin (Fluidigm 201198, 201194) for 2.5 minutes and washed in cRPMI. Cells were fixed with paraformaldehyde at a final concentration of 1.6% for 10 mins at RT in the dark and washed with Maxpar PBS (Fluidigm). Palladium mass-tag barcoding was performed as previously described using combinations of 6 palladium isotopes with Cell-ID 20-plex Pd barcoding kit (Fluidigm).1,2 All barcoded cells were then combined and washed with Maxpar cell staining buffer (CSB) (Fluidigm) and stained first with anti-CD16/32-159Tb for 30 min, followed by the rest of the surface antibody cocktail for 30 mins (Supplemental Table X). Cells were then washed with PBS, and permeabilized with ice cold 100% methanol (Fischer Scientific) for at least 30 mins. Following permeabilization, cell were washed and pelleted twice with CSB followed by intracellular antibody staining for 30 mins. Just prior to data collection, cells were stained with 250 nM Cell-ID Iridium intercalator (Fluidigm) in PBS with 1.6% PFA for 30 mins at 4° C. and then washed per protocol. Cells were then washed and rehydrated in double deionized water and collected on a Helios mass cytometer (Fluidigm). Barcorded .fcs files were normalized and debarcoded with Fluidigm Debarcoder software. Files were downloaded and gated in Cytobank (BeckmanCoulter) for single intercalator positive cells. CD45.2-positive cells were gated and files were downloaded and loaded into R for use by the CATALYST package.3,4 Analysis and plots were created using this following standard workflows.

mRNA Electroporation.

Lineage negative bone marrow cells were isolated using the Mouse Hematopoietic Progenitor Cell Isolation Kit (StemCell 19856) and electroporated with a custom Dre mRNA (Tri-Link) using the Thermo Neon electroporation device according to the manufacturer's protocol (100 uL kit, Thermo MPK10025). In brief, lineage negative bone marrow cells were resuspended in 153 ul of buffer T and 17 ul of Dre mRNA (1 ug/ul) was added and mixed by pipetting. Immediately after RNA addition, cells were electroporated using one pulse of 1700V for 20 ms. Following electroporation, cells were added to serum free media (StemSpan) with 50 ng SCF and 10 ng IL3 without antibiotics. For transplantation experiments, cells were incubated at 37 degrees for 1 hour and then injected into mice as described above. For extended culture, antibiotics were added 4 hours after electroporation.

### Bioinformatic Analysis

FASTQ files were demultiplexed using a java script from Takara. FASTQ files were mapped and transcript counts were enumerated using STAR (genome version mm10 and transcript version ensembl XX). Counts were input into R and RNA-sequencing analysis using DESeq2. Gene set enrichment analysis was performed using the fgsea package with genesets extracted from the msigdbr package. Single sample gene set enrichment analysis was performed using the gsva package. Figures were generated using ggplot2 and tidyheatmaps packages.

### Example 13: GOLDI-Lox On/Off Dre/Cre Npm1 Mouse

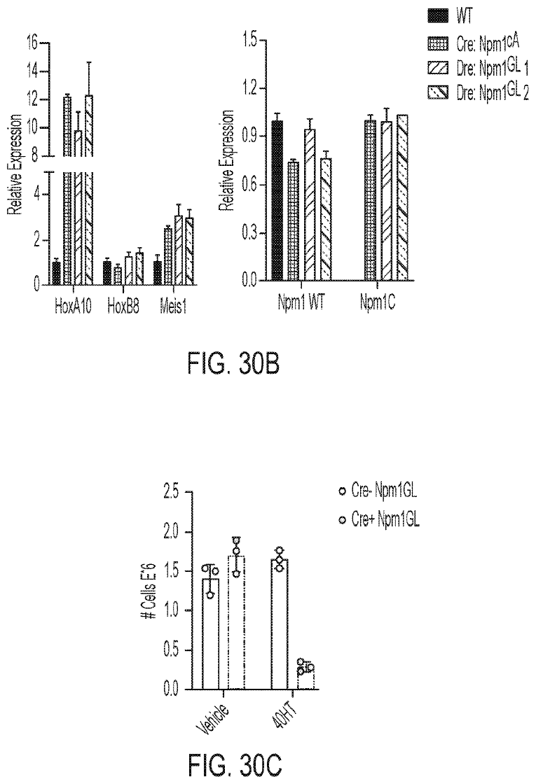

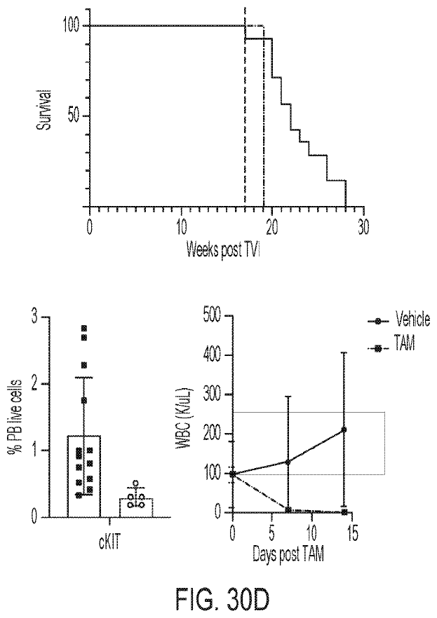

FIG. 30A shows a schematic of Npm1 GOLDI-Lox allele. At baseline, Npm1 wild-type (WT) exon 11 is expressed (Npm1 WT). Upon Dre-recombinase induction and inversion of the allele at the Rox sites, the WT exon is now on the reverse strand of DNA, and the humanized mutant Npm1 exon 11 (with TCTG duplication) is now expressed (i.e. mutant Npm1 or Npm1^{C}). A subsequent exposure to Cre recombinase causes a second inversion event at the Lox sites, leading to reversion to the Npm1 WT exon 11 (Npm1 WT).

The WT/mutant/WT functionality of the Npm1GOLDI-Lox allele was validated. Expression of mutant Npm1 has been previously shown to induce upregulation of the Hox/Meis loci. Additionally, the mutant Npm1 cDNA and protein harbor a unique C-terminal sequence which can be distinguished from Npm1 WT. Quantitative RT-PCR (qRT-PCR) was performed to determine expression levels of HoxA10, HoxB8, Meis1 (FIG. 30B, left panel) as well as expression levels of WT and mutant Npm1 (FIG. 30B, right panel) in hematopoietic stem and progenitor cells from WT (first bar from left), a previously established Cre-inducible Npm1 mouse (second bar from left), and two Dre-inducible Npm1 GOLDI-Lox mice (third and fourth bars from left) 7 days after Cre or Dre administration, respectively. Increased expression of Hox/Meis and mutant Npm1 transcripts was observed in all mutant cells with similar expression levels between Cre- and Dre- inducible lines.

FIG. 30C shows dependency of Npm1-mutant cells on expression of the mutant Npm1 protein. Based on previous data on mutant NPM1, it was expected that cells that lose the mutant Npm1 protein undergo differentiation and eventual apoptosis, ultimately leading to a loss of cell number. To validate the reversion of the mutant Npm1 GOLDI-Lox allele to WT, Dre-induced mutant Npm1 GOLDI-Lox hematopoietic cells were exposed to 4-hydroxytamoxifen (40HT) for Cre-recombinase induction, and cell number measured 7 days post 40HT administration using Cre⁻; Npm1GL cells as a negative control. A significant loss of cells was observed in Cre⁺; Npm1GL cells upon 40HT exposure, but a loss of cells was not observed in the Cre⁻; Npm1GL control cells.

FIG. 30D shows the development of disease with the Npm1 GOLDI-Lox allele. Previous data with established inducible Npm1 models (Cre-based) have confirmed that co-mutation with Nras^{G12D} leads to acute myeloid leukemia, with increased cKIT⁺ cells in the peripheral blood and severe leukocytosis (i.e. increased white blood cell (WBC) counts in moribund mice). A bone marrow transplant of a Dre-induced mutant Npm1 GOLDI-Lox expressing a co-mutant Nras^{G12D} allele immediately after Dre induction was performed, and mice were monitored for disease development (FIG. 30D, left panel). Upon development of disease, a cohort (n=5) of mice was treated with tamoxifen (TAM) to induce Cre and lead to reversion of the mutant Npm1 allele back to WT Npm1. While there was no significant difference in survival (FIG. 30D, left panel), there was a significant decrease in cKIT+ cells in the peripheral blood (FIG. 30D, middle panel) and a significant decrease in the WBC counts (FIG. 30D, right panel) in mice that received TAM compared to vehicle treated mice.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A nucleic acid comprising:
i) a first nucleic acid segment comprising, in 5′ to 3′ order:
a) a first member of a first pair of recombinase sites, each member of the first pair comprising a recognition sequence for a first recombinase;
b) a first member of a second pair of recombinase sites, each member of the second pair comprising a recognition sequence for a second recombinase;
c) a first member of a third pair of recombinase sites, each member of the third pair comprising a recognition sequence for the second recombinase; and
d) a second member of the first pair of recombinase sites;
ii) a second nucleic acid segment comprising an inverted expression cassette encoding a target gene; and
iii) a third nucleic acid segment comprising, in 5′ to 3′ order:
e) a second member of the second pair of recombinase sites; and
f) a second member of the third pair of recombinase sites,
wherein the nucleic acid comprises, in 5′ to 3′ order:
(i), (ii), and (iii); or
(iii), (ii), and (i).

2. The nucleic acid of claim 1, wherein (b)-(c) is not recombined upon exposure to the first recombinase.

3. The nucleic acid of claim 1 or 2, wherein (a) and (d) are separated from one another by a distance that prevents recombination of (b)-(c) upon exposure to the first recombinase.

4. The nucleic acid of any one of claims 1-3, wherein (a) and (d) are separated from one another by fewer than 90 contiguous nucleobases.

5. The nucleic acid of any one of claims 1-4, wherein (a) and (d) are separated from one another by fewer than 85 contiguous nucleobases.

6. The nucleic acid of any one of claims 1-5, wherein (a) and (d) are separated from one another by 82 contiguous nucleobases or fewer.

7. The nucleic acid of any one of claims 1-6, wherein the first and second members of each of the second and third pairs of recombinase sites are in an orientation opposite one another.

8. The nucleic acid of any one of claims 1-7, wherein the first and second members of each of the second and third pairs of recombinase sites are in forward and reverse orientations, respectively.

9. The nucleic acid of any one of claims 1-7, wherein the first and second members of each of the second and third pairs of recombinase sites are in reverse and forward orientations, respectively.

10. The nucleic acid of any one of claims 1-7, wherein:
the first and second members of the second pair of recombinase sites are in forward and reverse orientations, respectively; and
the first and second members of the third pair of recombinase sites are in reverse and forward orientations, respectively.

11. The nucleic acid of any one of claims 1-7, wherein:
the first and second members of the second pair of recombinase sites are in reverse and forward orientations, respectively; and
the first and second members of the third pair of recombinase sites are in forward and reverse orientations, respectively.

12. The nucleic acid of any one of claims 1-11, wherein:
the first recombinase is selected from Cre recombinase, Dre recombinase, and Flp recombinase; and
the second recombinase is selected from Cre recombinase, Dre recombinase, and Flp recombinase, provided that the first recombinase is different from the second recombinase.

13. The nucleic acid of any one of claims 1-12, wherein the first and second members of the first pair of recombinase sites are Lox sites, variant Lox sites, Frt sites, or variant Frt sites.

14. The nucleic acid of any one of claims 1-13, wherein:
the first and second members of the second pair of recombinase sites are Rox sites, and the first and second members of the third pair of recombinase sites are variant Rox sites; or
the first and second members of the second pair of recombinase sites are variant Rox sites, and the first and second members of the third pair of recombinase sites are Rox sites.

15. The nucleic acid of any one of claims 1-14, wherein the first and second members of each pair of recombinase sites are in an orientation opposite one another.

16. The nucleic acid of any one of claims 1-15, wherein the first and second members of the first pair of recombinase sites are in forward and reverse orientations, respectively.

17. The nucleic acid of any one of claims 1-15, wherein the first and second members of the first pair of recombinase sites are in reverse and forward orientations, respectively.

18. The nucleic acid of any one of claims 1-17, wherein the target gene encodes a protein of interest.

19. The nucleic acid of any one of claims 1-18, wherein the target gene encodes a protein comprising a disease-associated mutation.

20. The nucleic acid of any one of claims 1-14, wherein the second nucleic acid segment comprises:
a first expression cassette encoding a first target gene; and
a second expression cassette encoding a second target gene, wherein the first or second expression cassette is the inverted expression cassette.

21. The nucleic acid of claim 20, wherein:
the first target gene encodes a wild-type protein, and the second target gene encodes a mutational variant of the wild-type protein; or
the second target gene encodes a wild-type protein, and the first target gene encodes a mutational variant of the wild-type protein.

22. The nucleic acid of claim 20 or 21, wherein the first and second members of the first pair of recombinase sites are in the same orientation.

23. The nucleic acid of any one of claims 20-22, wherein the first and second members of the first pair of recombinase sites are variant Lox sites, each variant Lox site comprising a different sequence.

24. The nucleic acid of any one of claims 20-23, wherein the first pair of recombinase sites comprises a Lox66 site and a Lox71 site.

25. A cell comprising the nucleic acid of any one of claims 1-24.

26. The cell of claim 25, further comprising the first recombinase and/or a nucleic acid encoding the first recombinase.

27. The cell of claim 25 or 26, further comprising the second recombinase and/or a nucleic acid encoding the second recombinase.

28. The cell of any one of claims 25-27, further comprising an inducer that activates the first and/or second recombinase.

29. The cell of claim 28, wherein the inducer is a drug or chemical.

30. A non-human mammal comprising the nucleic acid of any one of claims 1-24 or the cell of any one of claims 25-29.

31. A nucleic acid comprising, in 5′ to 3′ order:
a) a first member of a first pair of recombinase sites, each member of the first pair comprising a recognition sequence for a first recombinase;
b) a first member of a second pair of recombinase sites, each member of the second pair comprising a recognition sequence for a second recombinase;
c) a first member of a third pair of recombinase sites, each member of the third pair comprising a recognition sequence for the second recombinase;
d) a second member of the first pair of recombinase sites;
e) an inverted expression cassette encoding a target gene;
f) a second member of the second pair of recombinase sites; and
g) a second member of the third pair of recombinase sites,
wherein the first and second members of each pair of recombinase sites are in an orientation opposite one another,
wherein (b)-(c) is not inverted upon exposure to the first recombinase, and wherein (c)-(e) or (d)-(f) is inverted upon exposure to the second recombinase.

32. The nucleic acid of claim 31, wherein the first and second members of the first pair of recombinase sites are in the same orientation upon inversion of (c)-(e) or (d)-(f) by the second recombinase.

33. The nucleic acid of claim 31 or 32, wherein (a) and (d) are separated from one another by a distance that prevents inversion of (b)-(c) upon exposure to the first recombinase.

34. The nucleic acid of any one of claims 31-33, wherein (a) and (d) are separated from one another by fewer than 90 contiguous nucleobases, fewer than 85 contiguous nucleobases, or 82 contiguous nucleobases or fewer.

35. The nucleic acid of any one of claims 31-34, wherein the first member of each pair of recombinase sites is in a forward orientation, and the second member of each pair of recombinase sites is in a reverse orientation.

36. The nucleic acid of any one of claims 31-34, wherein the first member of each pair of recombinase sites is in a reverse orientation, and the second member of each pair of recombinase sites is in a forward orientation.

37. The nucleic acid of any one of claims 31-36, wherein:
the first recombinase is selected from Cre recombinase, Dre recombinase, and Flp recombinase; and
the second recombinase is selected from Cre recombinase, Dre recombinase, and Flp recombinase, provided that the first recombinase is different from the second recombinase.

38. The nucleic acid of any one of claims 31-37, wherein the first and second members of the first pair of recombinase sites are Lox sites, variant Lox sites, Frt sites, or variant Frt sites.

39. The nucleic acid of any one of claims 31-38, wherein:
the first and second members of the second pair of recombinase sites are Rox sites, and the first and second members of the third pair of recombinase sites are variant Rox sites; or
the first and second members of the second pair of recombinase sites are variant Rox sites, and the first and second members of the third pair of recombinase sites are Rox sites.

40. The nucleic acid of any one of claims 31-39, wherein the target gene encodes a protein of interest.

41. The nucleic acid of any one of claims 31-40, wherein the target gene encodes a protein comprising a disease-associated mutation.

42. A method for regulating expression of a target gene, the method comprising:
contacting the nucleic acid of any one of claims 31-41 with the second recombinase, wherein the target gene is inverted to a sense orientation by the second recombinase.

43. The method of claim 42, further comprising contacting the nucleic acid with the first recombinase, wherein the target gene is excised from the nucleic acid by the first recombinase.

44. The method of claim 42 or 43, wherein the contacting is in a cell.

45. The method of claim 44, wherein the cell is in a non-human mammal.

46. A nucleic acid comprising, in 5′ to 3′ order:
a) a first member of a first pair of recombinase sites, each member of the first pair comprising a recognition sequence for a first recombinase;
b) a first member of a second pair of recombinase sites, each member of the second pair comprising a recognition sequence for the first recombinase;
c) an inverted expression cassette encoding a target gene;
d) a first member of a third pair of recombinase sites, each member of the third pair comprising a recognition sequence for a second recombinase;
e) a second member of the first pair of recombinase sites;
f) a second member of the second pair of recombinase sites; and
g) a second member of the third pair of recombinase sites,
wherein the first and second members of each pair of recombinase sites are in an orientation opposite one another,
wherein (e)-(f) is not inverted upon exposure to the second recombinase, and
wherein (b)-(d) or (c)-(e) is inverted upon exposure to the first recombinase.

47. The nucleic acid of claim 46, wherein the first and second members of the third pair of recombinase sites are in the same orientation upon inversion of (b)-(d) or (c)-(e) by the second recombinase.

48. The nucleic acid of claim 46 or 47, wherein (d) and (g) are separated from one another by a distance that prevents inversion of (e)-(f) upon exposure to the second recombinase.

49. The nucleic acid of any one of claims 46-48, wherein (d) and (g) are separated from one another by fewer than 90 contiguous nucleobases, fewer than 85 contiguous nucleobases, or 82 contiguous nucleobases or fewer.

50. The nucleic acid of any one of claims 46-49, wherein the first member of each pair of recombinase sites is in a forward orientation, and the second member of each pair of recombinase sites is in a reverse orientation.

51. The nucleic acid of any one of claims 46-49, wherein the first member of each pair of recombinase sites is in a reverse orientation, and the second member of each pair of recombinase sites is in a forward orientation.

52. The nucleic acid of any one of claims 46-51, wherein:
the first recombinase is selected from Cre recombinase, Dre recombinase, and Flp recombinase; and
the second recombinase is selected from Cre recombinase, Dre recombinase, and Flp recombinase, provided that the first recombinase is different from the second recombinase.

53. The nucleic acid of any one of claims 46-52, wherein the first and second members of the third pair of recombinase sites are Lox sites, variant Lox sites, Frt sites, or variant Frt sites.

54. The nucleic acid of any one of claims 46-53, wherein:
the first and second members of the first pair of recombinase sites are Rox sites, and the first and second members of the second pair of recombinase sites are variant Rox sites; or
the first and second members of the first pair of recombinase sites are variant Rox sites, and the first and second members of the second pair of recombinase sites are Rox sites.

55. The nucleic acid of any one of claims 46-54, wherein the target gene encodes a protein of interest.

56. The nucleic acid of any one of claims 46-55, wherein the target gene encodes a protein comprising a disease-associated mutation.

57. A method for regulating expression of a target gene, the method comprising:
contacting the nucleic acid of any one of claims 46-56 with the first recombinase, wherein the target gene is inverted to a sense orientation by the first recombinase.

58. The method of claim 57, further comprising contacting the nucleic acid with the second recombinase, wherein the target gene is excised from the nucleic acid by the second recombinase.

59. The method of claim 57 or 58, wherein the contacting is in a cell.

60. The method of claim 59, wherein the cell is in a non-human mammal.

61. A nucleic acid comprising, in 5′ to 3′ order:
a) a first member of a first pair of recombinase sites, each member of the first pair comprising a recognition sequence for a first recombinase;
b) a first member of a second pair of recombinase sites, each member of the second pair comprising a recognition sequence for a second recombinase;
c) a first member of a third pair of recombinase sites, each member of the third pair comprising a recognition sequence for the second recombinase;
d) a second member of the first pair of recombinase sites;
e) a first expression cassette encoding a first target gene;
f) a second expression cassette encoding a second target gene, wherein the first or second expression cassette is an inverted expression cassette;
g) a second member of the second pair of recombinase sites; and
h) a second member of the third pair of recombinase sites,
wherein the first and second members of the first pair of recombinase sites are in the same orientation,
wherein the first and second members of each of the second and third pairs of recombinase sites are in an orientation opposite one another,
wherein (b)-(c) is not excised from the nucleic acid upon exposure to the first recombinase, and
wherein (c)-(f) or (d)-(g) is inverted upon exposure to the second recombinase.

62. The nucleic acid of claim 61, wherein the first and second members of the first pair of recombinase sites are in an orientation opposite one another upon inversion of (c)-(f) or (d)-(g) by the second recombinase.

63. The nucleic acid of claim 61 or 62, wherein (a) and (d) are separated from one another by a distance that prevents excision of (b)-(c) upon exposure to the first recombinase.

64. The nucleic acid of any one of claims 61-63, wherein (a) and (d) are separated from one another by fewer than 90 contiguous nucleobases, fewer than 85 contiguous nucleobases, or 82 contiguous nucleobases or fewer.

65. The nucleic acid of any one of claims 61-64, wherein the first and second members of the first pair of recombinase sites are in a forward orientation.

66. The nucleic acid of any one of claims 61-64, wherein the first and second members of the first pair of recombinase sites are in a reverse orientation.

67. The nucleic acid of any one of claims 61-66, wherein:
the first recombinase is selected from Cre recombinase, Dre recombinase, and Flp recombinase; and
the second recombinase is selected from Cre recombinase, Dre recombinase, and Flp recombinase, provided that the first recombinase is different from the second recombinase.

68. The nucleic acid of any one of claims 61-67, wherein the first and second members of the first pair of recombinase sites are Lox sites, variant Lox sites, Frt sites, or variant Frt sites.

69. The nucleic acid of any one of claims 61-68, wherein the first and second members of the first pair of recombinase sites are variant Lox sites, each variant Lox site comprising a different sequence.

70. The nucleic acid of any one of claims 61-69, wherein the first pair of recombinase sites comprises a Lox66 site and a Lox71 site.

71. The nucleic acid of any one of claims 61-70, wherein:
the first and second members of the second pair of recombinase sites are Rox sites, and the first and second members of the third pair of recombinase sites are variant Rox sites; or
the first and second members of the second pair of recombinase sites are variant Rox sites, and the first and second members of the third pair of recombinase sites are Rox sites.

72. The nucleic acid of any one of claims 61-71, wherein:
the first target gene encodes a wild-type protein, and the second target gene encodes a mutational variant of the wild-type protein; or
the second target gene encodes a wild-type protein, and the first target gene encodes a mutational variant of the wild-type protein.

73. The nucleic acid of any one of claims 61-72, wherein the first or second target gene encodes a protein comprising a disease-associated mutation.

74. A method for regulating expression of a target gene, the method comprising:
contacting the nucleic acid of any one of claims 61-73 with the second recombinase, wherein the first or second target gene is inverted to a sense orientation by the second recombinase.

75. The method of claim 74, further comprising contacting the nucleic acid with the first recombinase, wherein the first or second target gene is inverted to an antisense orientation by the first recombinase.

76. The method of claim 74 or 75, wherein the contacting is in a cell.

77. The method of claim 76, wherein the cell is in a non-human mammal.

78. A nucleic acid comprising, in 5′ to 3′ order:
a) a first member of a first pair of recombinase sites, each member of the first pair comprising a recognition sequence for a first recombinase;
b) a first member of a second pair of recombinase sites, each member of the second pair comprising a recognition sequence for the first recombinase;
c) a first expression cassette encoding a first target gene;
d) a second expression cassette encoding a second target gene, wherein the first or second expression cassette is an inverted expression cassette;
e) a first member of a third pair of recombinase sites, each member of the third pair comprising a recognition sequence for a second recombinase;
f) a second member of the first pair of recombinase sites;
g) a second member of the second pair of recombinase sites; and
h) a second member of the third pair of recombinase sites,
wherein the first and second members of each of the first and second pairs of recombinase sites are in an orientation opposite one another,
wherein the first and second members of the third pair of recombinase sites are in the same orientation,
wherein (f)-(g) is not excised from the nucleic acid upon exposure to the second recombinase, and
wherein (b)-(e) or (c)-(f) is inverted upon exposure to the first recombinase.

79. The nucleic acid of claim 78, wherein the first and second members of the third pair of recombinase sites are in an orientation opposite one another upon inversion of (b)-(e) or (c)-(f) by the second recombinase.

80. The nucleic acid of claim 78 or 79, wherein (e) and (h) are separated from one another by a distance that prevents inversion of (f)-(g) upon exposure to the second recombinase.

81. The nucleic acid of any one of claims 78-80, wherein (e) and (h) are separated from one another by fewer than 90 contiguous nucleobases, fewer than 85 contiguous nucleobases, or 82 contiguous nucleobases or fewer.

82. The nucleic acid of any one of claims 78-81, wherein the first and second members of the third pair of recombinase sites are in a forward orientation.

83. The nucleic acid of any one of claims 78-81, wherein the first and second members of the third pair of recombinase sites are in a reverse orientation.

84. The nucleic acid of any one of claims 78-83, wherein:
the first recombinase is selected from Cre recombinase, Dre recombinase, and Flp recombinase; and
the second recombinase is selected from Cre recombinase, Dre recombinase, and Flp recombinase, provided that the first recombinase is different from the second recombinase.

85. The nucleic acid of any one of claims 78-84, wherein the first and second members of the third pair of recombinase sites are Lox sites, variant Lox sites, Frt sites, or variant Frt sites.

86. The nucleic acid of any one of claims 78-85, wherein the first and second members of the third pair of recombinase sites are variant Lox sites, each variant Lox site comprising a different sequence.

87. The nucleic acid of any one of claims 78-86, wherein the third pair of recombinase sites comprises a Lox66 site and a Lox71 site.

88. The nucleic acid of any one of claims 78-87, wherein:
the first and second members of the first pair of recombinase sites are Rox sites, and the first and second members of the second pair of recombinase sites are variant Rox sites; or
the first and second members of the first pair of recombinase sites are variant Rox sites, and the first and second members of the second pair of recombinase sites are Rox sites.

89. The nucleic acid of any one of claims 78-88, wherein:
the first target gene encodes a wild-type protein, and the second target gene encodes a mutational variant of the wild-type protein; or
the second target gene encodes a wild-type protein, and the first target gene encodes a mutational variant of the wild-type protein.

90. The nucleic acid of any one of claims 78-89, wherein the first or second target gene encodes a protein comprising a disease-associated mutation.

91. A method for regulating expression of a target gene, the method comprising:
contacting the nucleic acid of any one of claims 78-90 with the first recombinase, wherein the first or second target gene is inverted to a sense orientation by the first recombinase.

92. The method of claim 91, further comprising contacting the nucleic acid with the second recombinase, wherein the first or second target gene is inverted to an antisense orientation by the second recombinase.

93. The method of claim 91 or 92, wherein the contacting is in a cell.

94. The method of claim 93, wherein the cell is in a non-human mammal.

95. A cell comprising the nucleic acid of any one of claims 31-41, 46-56, 61-73, or 78-90.

96. The cell of claim 95, wherein the cell further comprises the first recombinase and/or a nucleic acid encoding the first recombinase.

97. The cell of claim 95 or 96, wherein the cell further comprises the second recombinase and/or a nucleic acid encoding the second recombinase.

98. The cell of any one of claims 95-97, wherein the first and/or second recombinase is activated by an inducer.

99. The cell of claim 98, wherein the inducer is a drug or chemical.

100. A non-human mammal comprising:
the nucleic acid of any one of claims 31-41, 46-56, 61-73, or 78-90; or
the cell of any one of claims 95-99.


SEQUENCE LISTING (91 sequences)

SEQ ID NO: 1 - DNA, 13 bp, unidentified
  source 1..13: mol_type = genomic DNA; note = Enterobacteria phage P1
ataacttcgtata

SEQ ID NO: 2 - DNA, 13 bp, unidentified
  source 1..13: mol_type = genomic DNA; note = Enterobacteria phage P1
tatacgaagttat

SEQ ID NO: 3 - DNA, 13 bp, synthetic construct
  source 1..13: mol_type = other DNA
ataacttcgtata

SEQ ID NO: 4 - DNA, 13 bp, synthetic construct
  source 1..13: mol_type = other DNA
tatacgaagttat

SEQ ID NO: 5 - DNA, 13 bp, synthetic construct
  source 1..13: mol_type = other DNA
ataacttcgtata

SEQ ID NO: 6 - DNA, 13 bp, synthetic construct
  source 1..13: mol_type = other DNA
tatacgaagttat

SEQ ID NO: 7 - DNA, 13 bp, synthetic construct
  source 1..13: mol_type = other DNA
ataacttcgtata

SEQ ID NO: 8 - DNA, 13 bp, synthetic construct
  source 1..13: mol_type = other DNA
tatacgaagttat

SEQ ID NO: 9 - DNA, 13 bp, synthetic construct
  source 1..13: mol_type = other DNA
taccgttcgtata

SEQ ID NO: 10 - DNA, 13 bp, synthetic construct
  source 1..13: mol_type = other DNA
tatacgaagttat

SEQ ID NO: 11 - DNA, 13 bp, synthetic construct
  source 1..13: mol_type = other DNA
taccgttcgtata

SEQ ID NO: 12 - DNA, 13 bp, synthetic construct
  source 1..13: mol_type = other DNA
tatacgaacggta

SEQ ID NO: 13 - DNA, 13 bp, synthetic construct
  source 1..13: mol_type = other DNA
ataacttcgtata

SEQ ID NO: 14 - DNA, 13 bp, synthetic construct
  source 1..13: mol_type = other DNA
tatacgaacggta

SEQ ID NO: 15 - DNA, 13 bp, synthetic construct
  source 1..13: mol_type = other DNA
ataacttcgtata

SEQ ID NO: 16 - DNA, 13 bp, synthetic construct
  source 1..13: mol_type = other DNA
tatacgaacggta

SEQ ID NO: 17 - DNA, 13 bp, synthetic construct
  source 1..13: mol_type = other DNA
ataaatctgtgga

SEQ ID NO: 18 - DNA, 13 bp, synthetic construct
  source 1..13: mol_type = other DNA
cacacaatcttag

SEQ ID NO: 19 - DNA, 13 bp, synthetic construct
  source 1..13: mol_type = other DNA
ataaatctgtgga

SEQ ID NO: 20 - DNA, 13 bp, synthetic construct
  source 1..13: mol_type = other DNA
tccacagatttat

SEQ ID NO: 21 - DNA, 13 bp, synthetic construct
  source 1..13: mol_type = other DNA
ctaagattgtgtg

SEQ ID NO: 22 - DNA, 13 bp, synthetic construct
  source 1..13: mol_type = other DNA
cacacaatcttag

SEQ ID NO: 23 - DNA, 13 bp, synthetic construct
  source 1..13: mol_type = other DNA
ataacttcatata

SEQ ID NO: 24 - DNA, 13 bp, synthetic construct
  source 1..13: mol_type = other DNA
tatatgaagttat

SEQ ID NO: 25 - DNA, 13 bp, synthetic construct
  source 1..13: mol_type = other DNA
ataacttcgtata

SEQ ID NO: 26 - DNA, 13 bp, synthetic construct
  source 1..13: mol_type = other DNA
tatacgaagttat

SEQ ID NO: 27 - DNA, 13 bp, synthetic construct
  source 1..13: mol_type = other DNA
ataacttcgtata

SEQ ID NO: 28 - DNA, 13 bp, synthetic construct
  source 1..13: mol_type = other DNA
tatacgaagttat

SEQ ID NO: 29 - DNA, 13 bp, synthetic construct
  source 1..13: mol_type = other DNA
ataactttgtata

SEQ ID NO: 30 - DNA, 13 bp, synthetic construct
  source 1..13: mol_type = other DNA
tatacaaagttat

SEQ ID NO: 31 - DNA, 13 bp, synthetic construct
  source 1..13: mol_type = other DNA
ataacttcgtgca

SEQ ID NO: 32 - DNA, 13 bp, synthetic construct
  source 1..13: mol_type = other DNA
tgcacgaagttat

SEQ ID NO: 33 - DNA, 13 bp, synthetic construct
  source 1..13: mol_type = other DNA
ataactctgtata

SEQ ID NO: 34 - DNA, 13 bp, synthetic construct
  source 1..13: mol_type = other DNA
tatacagagttat

SEQ ID NO: 35 - DNA, 13 bp, synthetic construct
  source 1..13: mol_type = other DNA
ataacttcgtata

SEQ ID NO: 36 - DNA, 13 bp, synthetic construct
  source 1..13: mol_type = other DNA
tatacgaagttat

SEQ ID NO: 37 - DNA, 13 bp, synthetic construct
  source 1..13: mol_type = other DNA
ataactctgtgta

SEQ ID NO: 38 - DNA, 13 bp, synthetic construct
  source 1..13: mol_type = other DNA
tacacagagttat

SEQ ID NO: 39 - DNA, 13 bp, synthetic construct
  source 1..13: mol_type = other DNA
taccgttcgtata

SEQ ID NO: 40 - DNA, 13 bp, synthetic construct
  source 1..13: mol_type = other DNA
tatacgaagttat

SEQ ID NO: 41 - DNA, 12 bp, synthetic construct
  source 1..12: mol_type = other DNA
taactttaaata

SEQ ID NO: 42 - DNA, 12 bp, synthetic construct
  source 1..12: mol_type = other DNA
tatttaaagtta

SEQ ID NO: 43 - DNA, 12 bp, synthetic construct
  source 1..12: mol_type = other DNA
taactttaaata

SEQ ID NO: 44 - DNA, 12 bp, synthetic construct
  source 1..12: mol_type = other DNA
tatttaaagtta

SEQ ID NO: 45 - DNA, 12 bp, synthetic construct
  source 1..12: mol_type = other DNA
taactttaaata

SEQ ID NO: 46 - DNA, 12 bp, synthetic construct
  source 1..12: mol_type = other DNA
tatttaaagtta

SEQ ID NO: 47 - DNA, 12 bp, synthetic construct
  source 1..12: mol_type = other DNA
taactttaaata

SEQ ID NO: 48 - DNA, 12 bp, synthetic construct
  source 1..12: mol_type = other DNA
tatttaaagtta

SEQ ID NO: 49 - DNA, 12 bp, synthetic construct
  source 1..12: mol_type = other DNA
taactttaaata

SEQ ID NO: 50 - DNA, 12 bp, synthetic construct
  source 1..12: mol_type = other DNA
tatttaaagtta

SEQ ID NO: 51 - DNA, 12 bp, synthetic construct
  source 1..12: mol_type = other DNA
taactttaaata

SEQ ID NO: 52 - DNA, 12 bp, synthetic construct
  source 1..12: mol_type = other DNA
tatttaaagtta

SEQ ID NO: 53 - DNA, 13 bp, synthetic construct
  source 1..13: mol_type = other DNA
gaagttcctattc

SEQ ID NO: 54 - DNA, 13 bp, synthetic construct
  source 1..13: mol_type = other DNA
gtataggaacttc

SEQ ID NO: 55 - DNA, 13 bp, synthetic construct
  source 1..13: mol_type = other DNA
gaagttcctattc

SEQ ID NO: 56 - DNA, 13 bp, synthetic construct
  source 1..13: mol_type = other DNA
gtataggaacttc

SEQ ID NO: 57 - DNA, 13 bp, synthetic construct
  source 1..13: mol_type = other DNA
gaagttcctattc

SEQ ID NO: 58 - DNA, 13 bp, synthetic construct
  source 1..13: mol_type = other DNA
gtataggaacttc

SEQ ID NO: 59 - DNA, 13 bp, synthetic construct
  source 1..13: mol_type = other DNA
gaagttcctattc

SEQ ID NO: 60 - DNA, 13 bp, synthetic construct
  source 1..13: mol_type = other DNA
gtataggaacttc

SEQ ID NO: 61 - DNA, 13 bp, synthetic construct
  source 1..13: mol_type = other DNA
gaagttcctattc

SEQ ID NO: 62 - DNA, 13 bp, synthetic construct
  source 1..13: mol_type = other DNA
gtataggaacttc

SEQ ID NO: 63 - DNA, 13 bp, synthetic construct
  source 1..13: mol_type = other DNA
gaagttcctattc

SEQ ID NO: 64 - DNA, 13 bp, synthetic construct
  source 1..13: mol_type = other DNA
gtataggaacttc

SEQ ID NO: 65 - DNA, 13 bp, synthetic construct
  source 1..13: mol_type = other DNA
gaagttcctattc

SEQ ID NO: 66 - DNA, 13 bp, synthetic construct
  source 1..13: mol_type = other DNA
gtataggaacttc

SEQ ID NO: 67 - DNA, 13 bp, synthetic construct
  source 1..13: mol_type = other DNA
gaagttcctattc

SEQ ID NO: 68 - DNA, 13 bp, synthetic construct
  source 1..13: mol_type = other DNA
gtataggaacttc

SEQ ID NO: 69 - DNA, 13 bp, synthetic construct
  source 1..13: mol_type = other DNA
gaagttcctattc

SEQ ID NO: 70 - DNA, 13 bp, synthetic construct
  source 1..13: mol_type = other DNA
ctataggaacttc

SEQ ID NO: 71 - DNA, 13 bp, synthetic construct
  source 1..13: mol_type = other DNA
gaagttcctatag

SEQ ID NO: 72 - DNA, 13 bp, synthetic construct
  source 1..13: mol_type = other DNA
gtataggaacttc

SEQ ID NO: 73 - DNA, 13 bp, synthetic construct
  source 1..13: mol_type = other DNA
gaagttcctattc

SEQ ID NO: 74 - DNA, 13 bp, synthetic construct
  source 1..13: mol_type = other DNA
ctatagaaacttc

SEQ ID NO: 75 - DNA, 13 bp, synthetic construct
  source 1..13: mol_type = other DNA
gaagtttctatag

SEQ ID NO: 76 - DNA, 13 bp, synthetic construct
  source 1..13: mol_type = other DNA
gtatagaaacttc

SEQ ID NO: 77 - DNA, 13 bp, synthetic construct
  source 1..13: mol_type = other DNA
gaagtttctattc

SEQ ID NO: 78 - DNA, 13 bp, synthetic construct
  source 1..13: mol_type = other DNA
gtaaaggaattag

SEQ ID NO: 79 - DNA, 13 bp, synthetic construct
  source 1..13: mol_type = other DNA
agtgatttgatac

SEQ ID NO: 80 - DNA, 13 bp, synthetic construct
  source 1..13: mol_type = other DNA
gaataggaacttc

SEQ ID NO: 81 - DNA, 13 bp, synthetic construct
  source 1..13: mol_type = other DNA
gaagttcctatac

SEQ ID NO: 82 - DNA, 164 bp, synthetic construct
  source 1..164: mol_type = other DNA
gtcgacataacttcgtataaagtatcctatacgaagttatcgtaactttaaataatgccaattatttaaagttacaattgacgactaactttaaataaggcctgttatttaaagttacacataacttcgtataggatactttatacgaagttatgatcacgcgt

SEQ ID NO: 83 - DNA, 24 bp, synthetic construct
  source 1..24: mol_type = other DNA
gccatctttccagcctaaaattag

SEQ ID NO: 84 - DNA, 26 bp, synthetic construct
  source 1..26: mol_type = other DNA
tccaaagagtctgtaagtacagaact

SEQ ID NO: 85 - DNA, 23 bp, synthetic construct
  source 1..23: mol_type = other DNA
accagttgctccagggttacacg

SEQ ID NO: 86 - DNA, 26 bp, synthetic construct
  source 1..26: mol_type = other DNA
aggagcatcgatgactacatgatgag

SEQ ID NO: 87 - DNA, 24 bp, synthetic construct
  source 1..24: mol_type = other DNA
agactctccacggtctcatctacg

SEQ ID NO: 88 - DNA, 20 bp, synthetic construct
  source 1..20: mol_type = other DNA
aagaattgctacaggcctgc

SEQ ID NO: 89 - DNA, 20 bp, synthetic construct
  source 1..20: mol_type = other DNA
ttctttagcccttgctgagc

SEQ ID NO: 90 - DNA, 20 bp, synthetic construct
  source 1..20: mol_type = other DNA
tagagggagggggcataagt

SEQ ID NO: 91 - DNA, 13 bp, synthetic construct
  source 1..13: mol_type = other DNA
gtataggaacttc